(19) United States
(12) Patent Application Publication
(10) Pub. No.: US 2017/0112775 A1
(43) Pub. Date: Apr. 27, 2017

(54) SITU SELF-ASSEMBLING PRO-NANOPARTICLE COMPOSITIONS AND METHODS OF PREPARATION AND USE THEREOF

(71) Applicant: University of North Texas Health Science Center, Fort Worth, TX (US)

(72) Inventor: Xiaowei DONG, Bedford, TX (US)

(73) Assignee: University of North Texas Health Science Center, Fort Worth, TX (US)

(21) Appl. No.: 15/282,380

(22) Filed: Sep. 30, 2016

Related U.S. Application Data
(60) Provisional application No. 62/235,753, filed on Oct. 1, 2015.

(51) Int. Cl. A61K 9/51; A61K 31/427

(52) U.S. Cl. CPC A61K 9/51; A61K 31/427; A61K 9/5123; A61K 9/5192

(57) ABSTRACT

Pharmaceutical composition comprising a pharmaceutically acceptable oil phase, a surfactant, and a therapeutic agent are provided herein, wherein the composition is in the form of a pro-nanoparticle or a self-assembling nanoparticle. Additionally, these pharmaceutical compositions have high loading of the therapeutic agent. Also provided herein are methods of preparing the pharmaceutical compositions and methods using the compositions in the treatment of a patient.

This application claims benefit of priority to U.S. Provisional Application Ser. No. 62/235,753, filed Oct. 1, 2015, the entire contents of which are hereby incorporated by reference.

### BACKGROUND

Field of the Disclosure

The present disclosure relates generally to the fields of medicine and pharmaceutics. In particular, it relates to compositions and methods for making in situ self-assembly nanoparticles (ISNPs). More particularly, it related to compositions of lipid-based nanoparticles.

Description of Related Art

A wide variety of therapeutic agents require additional preparation such as nanoparticle formulations to provide adequate therapeutic activity of the therapeutic agent. Many drugs are hydrophobic in nature and thus require additives to increase the ability of the agents to be dissolved in vivo. While useful for increasing the therapeutic activity, nanoparticles often have difficulty in formulation as the nanoparticles typically are not stable for long term storage, can lead to increased degradation of the therapeutic agent over time, and tend to aggregate reducing the effectiveness of the nanoparticles (Das and Chaudhury, 2011 and Abdelwahed, 2006). These issues with nanoparticle formulations hamper their use in a wide range of therapeutic applications. Furthermore, traditional nanoparticle compositions are not amenable to formulation as solid forms which can be administered as a capsule or tablet. Therefore, nanoparticle and pro-nanoparticle formulations which address aggregation, storage issues, and can be formulated in solid, parenteral, and topical formulations are of commercial interest.

### SUMMARY

In some aspects, the present disclosure provides compositions which act as pro-nanoparticles which when mixed with water or in vivo produce nanoparticles.

In yet another aspect, the present disclosure provides pharmaceutical compositions comprising:

(a) a therapeutic agent;

(b) a pharmaceutically acceptable oil phase; and

(c) a surfactant,

wherein the composition is formulated as a pro-nanoparticle formulation. In some embodiments, the pharmaceutically acceptable oil phase is a fatty acid or a mixture of a fatty acid. In some embodiments, the pharmaceutically acceptable oil phase is a fatty acid. In some embodiments, the pharmaceutically acceptable oil phase is an unsaturated fatty acid. In some embodiments, the pharmaceutically acceptable oil phase is a fatty acid comprising 6 carbon atom to 24 carbon atoms. In some embodiments, the pharmaceutically acceptable oil phase is oleic acid. The compositions may further comprise a pharmaceutically acceptable polymer, such as one that is a taste masking or taste improving agent.

In other embodiments, the pharmaceutically acceptable oil phase is a composition comprising one or more compounds of the formula:

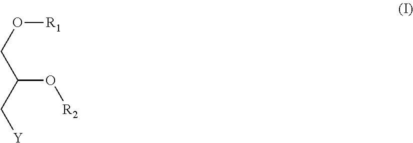

wherein:

R₁ and R₂ are each independently hydrogen or —C(O)—R₄; and

Y is hydrogen, hydroxy, or —OC(O)—R₄; wherein:

R₄ is alkyl_{(C1-25)}, alkenyl_{(C1-25)}, alkynyl_{(C1-25)}, or a substituted version of any of these groups; or a group of the formula: —C(O)—X—C(O)H, wherein X is an alkanediyl_{(C1-12)} or substituted alkanediyl_{(C1-12)};

provided that R₁, R₂, and Y are not all hydrogen. In some embodiments, R₁ or R₂ is —C(O)—R₄; wherein R₄ is alkyl_{(C4-18)}, alkenyl_{(C8-25)}, or alkynyl_{(C8-25)}. In some embodiments, R₄ is alkyl_{(C8-10)}. In some embodiments, the compound is Miglyol® 812. In other embodiments, the pharmaceutically acceptable oil phase is a naturally derived liquid oil. In some embodiments, the naturally derived liquid oil is corn oil, coconut oil, sunflowerseed oil, vegetable oil, cottonseed oil, mineral oil, peanut oil, sesame oil, soybean oil, or olive oil. In some embodiments, the surfactant has a hydrophilic-lipophilic balance (HLB) of from about 6 to about 20.

In some embodiments, the surfactant is a polyethylene glycol alkyl ether, polyethylene glycol vitamin derivative, a polyethylene glycol sorbitan fatty acid ester, a phospholipid, a polyethylene glycol stearate, a fatty alcohol, a lipophilic vitamin, or hexadecyltrimethylammonium bromide. In some embodiments, the surfactant is conjugated to a polyethylene glycol, a cell-targeting ligand, a small molecule, a peptide, a protein, or a carbohydrate. In some embodiments, the surfactant is a polyethylene glycol vitamin derivative. In some embodiments, the polyethylene glycol vitamin derivative is a PEGylated tocopheryl succinate. In some embodiments, the PEGylated tocopheryl succinate comprises a PEG group from about 100 g/mol to about 5,000 g/mol. In some embodiments, the PEGylated tocopheryl succinate is D-α-tocopheryl polyethylene glycol 1000 succinate.

In some embodiments, the surfactant further comprises a co-surfactant. In some embodiments, the pharmaceutically acceptable oil phase and the surfactant are present in a ratio from about 1:5 to about 5:1. In some embodiments, the ratio is from about 1:3 to about 3:1. In some embodiments, the ratio is about 1:1. In other embodiments, the ratio is about 1:2.

In some embodiments, the pharmaceutical composition comprises one therapeutic agent. In other embodiments, the pharmaceutical composition comprises a mixture of two or more therapeutic agents. In some embodiments, the therapeutic agent is a substantially water insoluble or lipophilic therapeutic agent. In some embodiments, the therapeutic agent is a water-soluble therapeutic agent. In some embodiments, the therapeutic agent is a small molecule, a therapeutic nucleic acid, or a therapeutic protein or peptide. In some embodiments, the small molecule is a chemotherapeutic, an anti-viral agent, a bacteriostatic or anti-bacterial agent, or an anti-fungal agent. In some embodiments, the therapeutic agent is a protease inhibitor. In some embodiments, the therapeutic agent is ritonavir. In other embodiments, the therapeutic agent is lopinavir. In other embodiments, the therapeutic agent is a mixture of ritonavir and lopinavir. In other embodiments, the therapeutic agent is a chemotherapeutic agent. In some embodiments, the therapeutic agent is docetaxel. In other embodiments, the therapeutic agent is a natural product. In some embodiments, the therapeutic agent is curcumin.

The therapeutic agent may be a mixture of ritonavir and lopinavir, a mixture of ritonavir and atazanavir, a mixture of atazanavir, ritonavir, emtricitabine and tenofovir disoproxil fumarate, a mixture of lopinavir, ritonavir, lamivudine and zidovudine, methotrexate, clindamycin, or a mixture of clindamycin and retinoic acid.

In some embodiments, the pharmaceutical composition further comprises an absorption agent. In some embodiments, the absorption agent is cellulose. In some embodiments, the absorption agent is Avicel® PH 102. In other embodiments, the absorption agent is silica. In some embodiments, the absorption agent is Aeroperl® 300.

In some embodiments, the pharmaceutical compositions are essentially free of water. In some embodiments, the pharmaceutical compositions are reconstituted into a liquid thereby forming a nanoparticle. In some embodiments, the liquid is water, saline, an injectable solution, juice, or other water based solution. In some embodiments, the liquid is salvia or another biological fluid such that the nanoparticle is formed in vivo.

In some embodiments, the therapeutic agent loading percentage of the nanoparticle is greater than 5%. In some embodiments, the therapeutic agent loading percentage of the nanoparticle is greater than 10%. In some embodiments, the therapeutic agent loading percentage of the nanoparticle is greater than 12.5%. In some embodiments, the nanoparticle has a mean diameter from about 100 nm to about 350 nm. In some embodiments, the nanoparticle has a mean diameter from about 150 nm to about 250 nm. In some embodiments, the nanoparticle has a mean diameter from about 100 nm to about 175 nm. In some embodiments, the nanoparticle has a polydispersity index from about 0.15 to about 0.325. In some embodiments, the nanoparticle has a polydispersity index from about 0.15 to about 0.25. In other embodiments, the nanoparticle has a polydispersity index from about 0.25 to about 0.325. In some embodiments, the nanoparticle has a drug entrapment efficiency is greater than 70%. In some embodiments, the nanoparticle has a drug entrapment efficiency is greater than 80%. In some embodiments, the nanoparticle has a drug entrapment efficiency is greater than 90%.

In some embodiments, the pharmaceutical compositions further comprise a pharmaceutically acceptable carrier. In some embodiments, the pharmaceutical compositions are formulated for oral, subcutaneous, intravenous, intraperitoneal, intratumoral, intraarterial, transdermal, topical, rectal, intranasal, transdermal, or buccal administration. In some embodiments, the pharmaceutical compositions further comprise formulating the composition for oral administration. In some embodiments, the formulations for oral administration are a fixed-dose formulation, a solid granule, an oral disintegrating tablet, an oral sustained release tablet, or an oral sustained release disintegrating tablet. In other embodiments, the pharmaceutical compositions further comprise formulating the composition for administration via an injection. In some embodiments, the formulations for administration via injection are formulated as a solid which may be reconstituted with saline or other pharmaceutically acceptable solutions.

In yet another aspect, the present disclosure provides methods of preparing a nanoparticle pharmaceutical composition comprising:
(a) admixing a pharmaceutically acceptable oil phase, a surfactant, and a therapeutic agent to form a first mixture; and
(b) heating the first mixture to a temperature from about 30° C. to about 100° C.
   In some embodiments, the temperature is from about 50° C. to about 75° C.

In still yet another aspect, the present disclosure provides methods of preparing an in-situ self assembly nanoparticle pharmaceutical composition comprising:
(a) admixing a therapeutic agent, a pharmaceutically acceptable oil phase, and a surfactant to form a first mixture;
(b) heating the first mixture to a temperature from about 30° C. to about 100° C. for a time period from about 1 minute to about 1 hour; and
(c) cooling the first mixture to room temperature.
   In some embodiments, the temperature is from about 50° C. to about 75° C. In some embodiments, the time period is from about 5 minutes to about 30 minutes.

In yet another aspect, the present disclosure provides methods of preparing a pharmaceutical composition comprising:
(a) admixing a therapeutic agent, a pharmaceutically acceptable oil phase, and a surfactant to form a first mixture;
(b) heating the first mixture to a temperature from about 30° C. to about 100° C. for a time period from about 1 minute to about 1 hour;
(c) admixing an absorption agent to the first mixture to obtain a second mixture;
(d) heating the second mixture to a temperature from about 30° C. to about 100° C. for a time period from about 1 minute to about 1 hour; and
(e) cooling the second mixture to room temperature.
   In some embodiments, the absorption agent is cellulose such as Avicel® PH 102. In other embodiments, the absorption agent is silica such as Aeroperl® 300. In some embodiments, the pharmaceutically acceptable oil phase is a fatty acid or a mixture of a fatty acid. In some embodiments, the pharmaceutically acceptable oil phase is a fatty acid. In some embodiments, the pharmaceutically acceptable oil phase is an unsaturated fatty acid. In some embodiments, the pharmaceutically acceptable oil phase is a fatty acid comprising 6 carbon atom to 24 carbon atoms. In some embodiments, the pharmaceutically acceptable oil phase is oleic acid. In other embodiments, the pharmaceutically acceptable oil phase is a composition comprising one or more compounds of the formula:

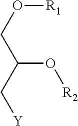

wherein:

R₁ and R₂ are each independently hydrogen or —C(O)—R₄; and

Y is hydrogen, hydroxy, or —OC(O)—R₄; wherein:
R₄ is alkyl_{(C1-25)}, alkenyl_{(C1-25)}, alkynyl_{(C1-25)}, or a substituted version of any of these groups; or a group of the formula: —C(O)—X—C(O)H, wherein X is an alkanediyl(C₁₋₁₂₎ or substituted alkanediyl(C₁₋₁₂₎;

provided that R₁, R₂, and Y are not all hydrogen.

In some embodiments, R₁ or R₂ is —C(O)—R₄; wherein R₄ is alkyl(C₄₋₁₈₎, alkenyl(C₈₋₂₅₎, or alkynyl(C₈₋₂₅₎. In some embodiments, R₄ is alkyl(C₈₋₁₀₎. In some embodiments, the compound is Miglyol® 812 In other embodiments, the pharmaceutically acceptable oil phase is a naturally derived liquid oil. In some embodiments, the naturally derived liquid oil is corn oil, coconut oil, sunflowerseed oil, vegetable oil, cottonseed oil, mineral oil, peanut oil, sesame oil, soybean oil, or olive oil. In some embodiments, the surfactant has a hydrophilic-lipophilic balance (HLB) of from about 6 to about 20.

In some embodiments, the surfactant is a polyethylene glycol alkyl ether, polyethylene glycol vitamin derivative, a polyethylene glycol sorbitan fatty acid ester, a phospholipid, a polyethylene glycol stearate, a fatty alcohol, a lipophilic vitamin, or hexadecyltrimethylammonium bromide. In some embodiments, the surfactant is conjugated to a polyethylene glycol, a cell-targeting ligand, a small molecule, a peptide, a protein, or a carbohydrate. In some embodiments, the surfactant is a polyethylene glycol vitamin derivative. In some embodiments, the polyethylene glycol vitamin derivative is a PEGylated tocopheryl succinate. In some embodiments, the PEGylated tocopheryl succinate comprises a PEG group from about 100 g/mol to about 5,000 g/mol. In some embodiments, the PEGylated tocopheryl succinate is D-α-tocopheryl polyethylene glycol 1000 succinate.

In some embodiments, the surfactant further comprises a co-surfactant. In some embodiments, the pharmaceutically acceptable oil phase and the surfactant are present in a ratio from about 1:5 to about 5:1. In some embodiments, the ratio is from about 1:3 to about 3:1. In some embodiments, the ratio is about 1:1. In other embodiments, the ratio is about 1:2.

In some embodiments, the pharmaceutical composition comprises one therapeutic agent. In other embodiments, the pharmaceutical composition comprises a mixture of two or more therapeutic agents. In some embodiments, the therapeutic agent is a substantially water insoluble or lipophilic therapeutic agent. In some embodiments, the therapeutic agent is a water-soluble therapeutic agent. In some embodiments, the therapeutic agent is a small molecule, a therapeutic nucleic acid, or a therapeutic protein or peptide. In some embodiments, the small molecule is a chemotherapeutic, an anti-viral agent, a bacteriostatic or anti-bacterial agent, or an anti-fungal agent. In some embodiments, the therapeutic agent is a protease inhibitor. In some embodiments, the therapeutic agent is ritonavir. In other embodiments, the therapeutic agent is lopinavir. In other embodiments, the therapeutic agent is a mixture of ritonavir and lopinavir. In other embodiments, the therapeutic agent is a chemotherapeutic agent. In some embodiments, the therapeutic agent is docetaxel. In other embodiments, the therapeutic agent is a natural product. In some embodiments, the therapeutic agent is curcumin.

In some embodiments, the methods further comprise adding a pharmaceutically acceptable carrier. In some embodiments, the methods further comprise formulating the composition for oral, subcutaneous, intravenous, intraperitoneal, intratumoral, intraarterial, transdermal, topical, rectal, intranasal, transdermal, or buccal administration. In some embodiments, the methods further comprise formulating the composition for oral administration. In some embodiments, the formulations for oral administration are a fixed-dose formulation, a solid granule, an oral disintegrating tablet, an oral sustained release tablet, or an oral sustained release disintegrating tablet. In other embodiments, the methods further comprise formulating the composition for administration via an injection. In some embodiments, the formulations for administration via injection are formulated as a solid which may be reconstituted with saline or other pharmaceutically acceptable solutions.

In still yet another aspect, the present disclosure provides pharmaceutical compositions prepared according to the methods described herein.

In yet another aspect, the present disclosure provides methods of treating a disease or disorder comprising administering to a patient in need thereof a therapeutically effective amount of a pharmaceutical composition described herein. In some embodiments, the therapeutic agent is a small molecule, a therapeutic nucleic acid, or a therapeutic protein or peptide. In some embodiments, the small molecule is a chemotherapeutic, an anti-viral agent, a bacteriostatic or anti-bacterial agent, or an anti-fungal agent. In some embodiments, the therapeutic agent is a protease inhibitor. In some embodiments, the therapeutic agent is ritonavir. In other embodiments, the therapeutic agent is lopinavir. In other embodiments, the therapeutic agent is a mixture of ritonavir and lopinavir. In some embodiments, the disease or disorder is human immunodeficiency virus or acquired immunodeficienecy syndrome. In other embodiments, the therapeutic agent is a chemotherapeutic agent. In other embodiments, the therapeutic agent is docetaxel. In other embodiments, the disease or disorder is cancer. In some embodiments, the cancer is prostate cancer. In other embodiments, the therapeutic agent is a natural product. In other embodiments, the therapeutic agent is curcumin. In other embodiments, the disease or disorder is cancer. In some embodiments, the cancer is prostate cancer. In some embodiments, the patient is a mammal. In some embodiments, the patient is a human.

It is contemplated that any method or composition described herein can be implemented with respect to any other method or composition described herein.

The terms “comprise” (and any form of comprise, such as “comprises” and “comprising”), “have” (and any form of have, such as “has” and “having”), “contain” (and any form of contain, such as “contains” and “containing”), and “include” (and any form of include, such as “includes” and “including”) are open-ended linking verbs. As a result, a method, composition, kit, or system that “comprises,” “has,” “contains,” or “includes” one or more recited steps or elements possesses those recited steps or elements, but is not limited to possessing only those steps or elements; it may possess (i.e., cover) elements or steps that are not recited. Likewise, an element of a method, composition, kit, or system that “comprises,” “has,” “contains,” or “includes” one or more recited features possesses those features, but is not limited to possessing only those features; it may possess features that are not recited.

Any embodiment of any of the present methods, composition, kit, and systems may consist of or consist essentially of—rather than comprise/include/contain/have—the described steps and/or features. Thus, in any of the claims, the term “consisting of” or “consisting essentially of” may be substituted for any of the open-ended linking verbs recited above, in order to change the scope of a given claim from what it would otherwise be using the open-ended linking verb.

The use of the term “or” in the claims is used to mean “and/or” unless explicitly indicated to refer to alternatives only or the alternatives are mutually exclusive, although the disclosure supports a definition that refers to only alternatives and “and/or.”

Throughout this application, the term “about” is used to indicate that a value includes the standard deviation of error for the device or method being employed to determine the value.

Following long-standing patent law, the words “a” and “an,” when used in conjunction with the word “comprising” in the claims or specification, denotes one or more, unless specifically noted.

As used in this application, the term “average molecular weight” refers to the relationship between the number of moles of each polymer species and the molar mass of that species. In particular, each polymer molecule may have different levels of polymerization and thus a different molar mass. The average molecular weight can be used to represent the molecular weight of a plurality of polymer molecules. Average molecular weight is typically synonymous with average molar mass. In particular, there are three major types of average molecular weight: number average molar mass, weight (mass) average molar mass, and Z-average molar mass. In the context of this application, unless otherwise specified, the average molecular weight represents either the number average molar mass or weight average molar mass of the formula. In some embodiments, the average molecular weight is the number average molar mass. In some embodiments, the average molecular weight may be used to describe a PEG component present in the surfactant.

An “oil phase,” as used herein, refers to a lipid, polymer, or mixture thereof which provides a hydrophobic environment for self-assembly of nanoparticles. A wide variety of different lipids and polymers may be used in the present disclosure to form the oil phase, as described herein. In some embodiments, in situ self-assembly pro-nanoparticles compositions of the present disclosure may comprise a fatty acid, monoglyceride, a diglyceride, a triglyceride, a monoester or diester of propylene glycol, or the mixture of them.

Other objects, features and advantages of the present disclosure will become apparent from the following detailed description. It should be understood, however, that the detailed description and the specific examples, while indicating specific embodiments of the disclosure, are given by way of illustration only, since various changes and modifications within the spirit and scope of the disclosure will become apparent to those skilled in the art from this detailed description.

### BRIEF DESCRIPTION OF THE DRAWINGS

The following drawings form part of the present specification and are included to further demonstrate certain aspects of the present disclosure. The disclosure may be better understood by reference to one or more of these drawings in combination with the detailed description of specific embodiments presented herein.

The patent or application file contains at least one drawing executed in color. Copies of this patent or patent application publication with color drawing(s) will be provided by the Office upon request and payment of the necessary fee.

FIGS. 1A-B. The illustration of comparing the preparation and formation of (A) RTV nanoparticle-containing granules (prepared by a microemulsion method) and (B) RTV ISNP granules.

FIG. 2. Short-term stability of RTV, LPV and LPV/RTV ISNP solid granules in a simulated physiological condition (n=3).

FIGS. 3A-B. Release of (FIG. 3A) RTV and (FIG. 3B) LPV from the ISNP granules compared with RTV nanocapsules and a commercial LPV/RTV tablet (n=3).

FIG. 4. Particle size of prototype nanoparticles.

FIGS. 5A-B. In vitro taste assessment. (A) Drug release of LPV/RTV ISNP granules within 60 min. (B) Taste evaluation of RTV and LPV/RTV ISNP granules by using the E-tongue.

FIG. 6. PK study of RTV solid granules v.s. RTV commercial solution in rats (n=3).

FIG. 7. PK Study of LPV solid granules and LPV/RTV solid granules v.s. LPV/RTV commercial tablet in rats (n=3).

FIG. 8. PK Study of ATV/RTV/FTC/TDF FDC ISNP granules v.s. a combination of commercial drugs in rats (n=3).

FIG. 9. Biodistribution of LPV/RTV ISNP granules in rats (n=3).

FIG. 10. Caco-2 permeability of TDF ISNP granules (n=3). (#p<0.05)

FIG. 11. Particle size and size distribution of LPV/RTV ISNPs for long-acting injections.

FIG. 12. Release of LPV and RTV from LPV/RTV ISNPs for long-acting injections. The study was performed in PBS (pH 7.4) at 37° C. for 14 days. Released LPV and RTV were separated from LPV/RTV ISNPs by Microcon-Y100 and measured by HPLC. Data are presented as the mean±SD (n=3).

FIGS. 13A-B. Plasma concentration vs time profiles of (A) LPV and (B) RTV from a long-acting injection by subcutaneous injection. Rat were dosed using LPV/RTV ISNPs at 100 mg/kg of LPV by subcutaneous injective. Data are shown as the mean±SD (n=3).

FIG. 14. Long-term stability blank MT and DTX MT NPs. Three different batches of DTX MT NPs and blank NPs stored at 4° C. for over 6 months. All tested samples had PI<0.32.

FIG. 15. Stability of DTX MT NPs and blank NPs in PBS at 37° C. All tested NPs were monitored for particle size for 96 hr and had PI<0.30. Data are presented as the mean±SD of three separate measurement (n=3) of each batch.

FIG. 16. In vitro release profile of DTX MT NPs in PBS at 37° C. Data are presented as the mean±SD (n=3).

FIG. 17. Long-term stability of CUR MT NPs stored at 4° C. Three different batches of CUR MT NPs and blank NPs were monitored for particle sizes over six months. For all tested samples, P.I.<0.25. Data are presented as the mean particle size of three measurement of each batch.

FIG. 18. Physical stability of particle size for both CUR MT NPs and blank NPs in PBS (pH 7.4) at 37° C. for up to 96 hr. For all tested samples, P.I.<0.25. Data are presented as the mean±SD (n=3).

FIG. 19. Release of CUR from CUR MT NPs in PBS (pH 7.4) at 37° C. for 8 hr. Curcumin release was measured by recovering CUR NP in filtrate by using the reverse spinning method. For all tested samples, P.I.<0.25. Data are presented as the mean±SD (n=3).

FIG. 20. Schematic for application of the ISNP nanotechnology to oral, parenteral and topical formulations.

### DESCRIPTION OF ILLUSTRATIVE EMBODIMENTS

As described herein, in situ self-assembly nanoparticles (ISNPs) have been developed. By mixing one or more oils and one or more surfactants, pro-ISNP compositions were obtained which are not yet constituted into a nanoparticle yet. When these pro-ISNP compositions are introduced into a liquid (e.g. water, juice, or the fluid in gastrointestinal tract), the pro-ISNP compositions spontaneously form oil based NPs in situ. And because water has completely removed from the NP preparations, the drug-loaded pre-ISNP may be convert into solid granules by mixing them with adsorption carriers. Pharmacokinetic (PK) studies demonstrated that drug-loaded solid granules increased the bioavailability of the drugs compared to the controls. To the inventor's knowledge, the studies described herein are the first studies of a pro-nanoparticle formulation. This nanotechnology provides an advantageous formulation method for transforming poorly water-soluble drugs into flexible solid oral dosage forms. These and other aspects of the disclosure are described in detail below.

### A. CHEMICAL DEFINITIONS

When used in the context of a chemical group: “hydrogen” means —H; “hydroxy” means —OH; “oxo” means ═O; “carbonyl” means —C(═O)—; “carboxy” means —C(═O)OH (also written as —COOH or —CO₂H); “halo” means independently —F, —Cl, —Br or —I; “amino” means —NH₂; “hydroxyamino” means —NHOH; “nitro” means —NO₂; imino means ═NH; “cyano” means —CN; “isocyanate” means —N═C═O; “azido” means —N₃; in a monovalent context “phosphate” means —OP(O)(OH)₂ or a deprotonated form thereof; in a divalent context “phosphate” means —OP(O)(OH)O— or a deprotonated form thereof; “mercapto” means —SH; and “thio” means ═S; “sulfonyl” means —S(O)₂—; and “sulfinyl” means —S(O)—.

In the context of chemical formulas, the symbol “—” means a single bond, “=” means a double bond, and “≡” means triple bond. The symbol “----” represents an optional bond, which if present is either single or double. The symbol “ [symbol] ” represents a single bond or a double bond. Thus, for example, the formula

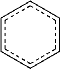

includes

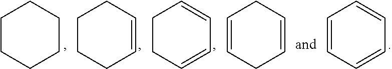

And it is understood that no one such ring atom forms part of more than one double bond. Furthermore, it is noted that the covalent bond symbol “—”, when connecting one or two stereogenic atoms, does not indicate any preferred stereochemistry. Instead, it cover all stereoisomers as well as mixtures thereof. The symbol “ [symbol] ”, when drawn perpendicularly across a bond (e.g.,

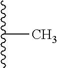

for methyl) indicates a point of attachment of the group. It is noted that the point of attachment is typically only identified in this manner for larger groups in order to assist the reader in unambiguously identifying a point of attachment. The symbol “ [symbol] ” means a single bond where the group attached to the thick end of the wedge is “out of the page.” The symbol “ [symbol] ” means a single bond where the group attached to the thick end of the wedge is “into the page”. The symbol “ [symbol] ” means a single bond where the geometry around a double bond (e.g., either E or Z) is undefined. Both options, as well as combinations thereof are therefore intended. Any undefined valency on an atom of a structure shown in this application implicitly represents a hydrogen atom bonded to that atom. A bold dot on a carbon atom indicates that the hydrogen attached to that carbon is oriented out of the plane of the paper.

When a group “R” is depicted as a “floating group” on a ring system, for example, in the formula:

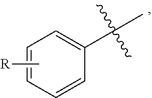

then R may replace any hydrogen atom attached to any of the ring atoms, including a depicted, implied, or expressly defined hydrogen, so long as a stable structure is formed. When a group “R” is depicted as a “floating group” on a fused ring system, as for example in the formula:

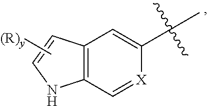

then R may replace any hydrogen attached to any of the ring atoms of either of the fused rings unless specified otherwise. Replaceable hydrogens include depicted hydrogens (e.g., the hydrogen attached to the nitrogen in the formula above), implied hydrogens (e.g., a hydrogen of the formula above that is not shown but understood to be present), expressly defined hydrogens, and optional hydrogens whose presence depends on the identity of a ring atom (e.g., a hydrogen attached to group X, when X equals —CH—), so long as a stable structure is formed. In the example depicted, R may reside on either the 5-membered or the 6-membered ring of the fused ring system. In the formula above, the subscript letter “y” immediately following the group “R” enclosed in parentheses, represents a numeric variable. Unless specified otherwise, this variable can be 0, 1, 2, or any integer greater than 2, only limited by the maximum number of replaceable hydrogen atoms of the ring or ring system.

For the groups and classes below, the number of carbon atoms in the group is as indicated as follows: “Cn” defines the exact number (n) of carbon atoms in the group/class. “Cn” defines the maximum number (n) of carbon atoms that can be in the group/class, with the minimum number as small as possible for the group in question, e.g., it is understood that the minimum number of carbon atoms in the group “alkenyl_{(C≦8)}” or the class “alkene_{(C≦8)}” is two. Compare with “alkoxy_{(C≦10)}”, which designates alkoxy groups having from 1 to 10 carbon atoms. Also compare “phosphine_{(C≦10)}”, which designates phosphine groups having from 0 to 10 carbon atoms. “Cn-n′” defines both the minimum (n) and maximum number (n′) of carbon atoms in the group. Thus, “alkyl_{(C2-10)}” designates those alkyl groups having from 2 to 10 carbon atoms. Typically the carbon number indicator follows the group it modifies, is enclosed with parentheses, and is written entirely in subscript; however, the indicator may also precede the group, or be written without parentheses, without signifying any change in meaning. Thus, the terms “C5 olefin”, “C5-olefin”, “olefin_{(C5)}”, and “olefincs” are all synonymous.

The term “saturated” as used herein means the compound or group so modified has no carbon-carbon double and no carbon-carbon triple bonds, except as noted below. In the case of substituted versions of saturated groups, one or more carbon oxygen double bond or a carbon nitrogen double bond may be present. And when such a bond is present, then carbon-carbon double bonds that may occur as part of keto-enol tautomerism or imine/enamine tautomerism are not precluded.

The term “aliphatic” when used without the “substituted” modifier signifies that the compound/group so modified is an acyclic or cyclic, but non-aromatic hydrocarbon compound or group. In aliphatic compounds/groups, the carbon atoms can be joined together in straight chains, branched chains, or non-aromatic rings (alicyclic). Aliphatic compounds/groups can be saturated, that is joined by single bonds (alkanes/alkyl), or unsaturated, with one or more double bonds (alkenes/alkenyl) or with one or more triple bonds (alkynes/alkynyl).

The term “alkyl” when used without the “substituted” modifier refers to a monovalent saturated aliphatic group with a carbon atom as the point of attachment, a linear or branched acyclic structure, and no atoms other than carbon and hydrogen. The groups —CH₃ (Me), —CH₂CH₃(Et), —CH₂CH₂CH₃ (n-Pr or propyl), —CH(CH₃)₂ (i-Pr, ⁱPr or isopropyl), —CH₂CH₂CH₂CH₃ (n-Bu), —CH(CH₃)CH₂CH₃ (sec-butyl), —CH₂CH(CH₃)₂ (isobutyl), —C(CH₃)₃ (tert-butyl, t-butyl, t-Bu or ^{t}Bu), and —CH₂C(CH₃)₃ (neo-pentyl) are non-limiting examples of alkyl groups. The term “alkanediyl” when used without the “substituted” modifier refers to a divalent saturated aliphatic group, with one or two saturated carbon atom(s) as the point(s) of attachment, a linear or branched acyclic structure, no carbon-carbon double or triple bonds, and no atoms other than carbon and hydrogen. The groups —CH₂— (methylene), —CH₂CH₂—, —CH₂C(CH₃)₂CH₂—, and —CH₂CH₂CH₂— are non-limiting examples of alkanediyl groups. The term “alkylidene” when used without the “substituted” modifier refers to the divalent group ═CRR′ in which R and R′ are independently hydrogen or alkyl. Non-limiting examples of alkylidene groups include: ═CH₂, ═CH(CH₂CH₃), and ═C(CH₃)₂. An “alkane” refers to the compound H—R, wherein R is alkyl as this term is defined above. When any of these terms is used with the “substituted” modifier one or more hydrogen atom has been independently replaced by —OH, —F, —Cl, —Br, —I, —NH₂, —NO₂, —CO₂H, —CO₂CH₃, —CN, —SH, —OCH₃, —OCH₂CH₃, —C(O)CH₃, —NHCH₃, —NHCH₂CH₃, —N(CH₃)₂, —C(O)NH₂, —C(O)NHCH₃, —C(O)N(CH₃)₂, —OC(O)CH₃, —S(O)₂₀H, or —S(O)₂NH₂. The following groups are non-limiting examples of substituted alkyl groups: —CH₂OH, —CH₂C₁, —CF₃, —CH₂CN, —CH₂C(O)OH, —CH₂C(O)OCH₃, —CH₂C(O)NH₂, —CH₂C(O)CH₃, —CH₂OCH₃, —CH₂OC(O)CH₃, —CH₂NH₂, —CH₂N(CH₃)₂, and —CH₂CH₂C₁. The term “haloalkyl” is a subset of substituted alkyl, in which the hydrogen atom replacement is limited to halo (i.e. —F, —Cl, —Br, or —I) such that no other atoms aside from carbon, hydrogen and halogen are present. The group, —CH₂Cl is a non-limiting example of a haloalkyl. The term “fluoroalkyl” is a subset of substituted alkyl, in which the hydrogen atom replacement is limited to fluoro such that no other atoms aside from carbon, hydrogen and fluorine are present. The groups —CH₂F, —CF₃, and —CH₂CF₃ are non-limiting examples of fluoroalkyl groups.

The term “alkenyl” when used without the “substituted” modifier refers to an monovalent unsaturated aliphatic group with a carbon atom as the point of attachment, a linear or branched acyclic structure, at least one nonaromatic carbon-carbon double bond, no carbon-carbon triple bonds, and no atoms other than carbon and hydrogen. Non-limiting examples include: —CH═CH₂ (vinyl), —CH═CHCH₃, —CH═CHCH₂CH₃, —CH₂CH═CH₂ (allyl), —CH₂CH═CHCH₃, and —CH═CHCH═CH₂. The term “alkenediyl” when used without the “substituted” modifier refers to a divalent unsaturated aliphatic group, with two carbon atoms as points of attachment, a linear or branched, a linear or branched acyclic structure, at least one nonaromatic carbon-carbon double bond, no carbon-carbon triple bonds, and no atoms other than carbon and hydrogen. The groups —CH═CH—, —CH═C(CH₃)CH₂—, —CH═CHCH₂—, and —CH₂CH═CHCH₂— are non-limiting examples of alkenediyl groups. It is noted that while the alkenediyl group is aliphatic, once connected at both ends, this group is not precluded from forming part of an aromatic structure. The terms “alkene” or “olefin” are synonymous and refer to a compound having the formula H—R, wherein R is alkenyl as this term is defined above. A “terminal alkene” refers to an alkene having just one carbon-carbon double bond, wherein that bond forms a vinyl group at one end of the molecule. When any of these terms are used with the “substituted” modifier one or more hydrogen atom has been independently replaced by —OH, —F, —Cl, —Br, —I, —NH₂, —NO₂, —CO₂H, —CO₂CH₃, —CN, —SH, —OCH₃, —OCH₂CH₃, —C(O)CH₃, —NHCH₃, —NHCH₂CH₃, —N(CH₃)₂, —C(O)NH₂, —C(O)NHCH₃, —C(O)N(CH₃)₂, —OC(O)CH₃, —S(O)₂OH, or —S(O)₂NH₂. The groups —CH═CHF, —CH═CHCl and —CH═CHBr are non-limiting examples of substituted alkenyl groups.

The term “alkynyl” when used without the “substituted” modifier refers to a monovalent unsaturated aliphatic group with a carbon atom as the point of attachment, a linear or branched acyclic structure, at least one carbon-carbon triple bond, and no atoms other than carbon and hydrogen. As used herein, the term alkynyl does not preclude the presence of one or more non-aromatic carbon-carbon double bonds. The groups —C≡CH, —C≡CCH₃, and —CH₂C≡CCH₃ are non-limiting examples of alkynyl groups. An “alkyne” refers to the class of compounds having the formula H—R, wherein R is alkynyl. When any of these terms are used with the “substituted” modifier one or more hydrogen atom has been independently replaced by —OH, —F, —Cl, —Br, —I, —NH₂, —NO₂, —CO₂H, —CO₂CH₃, —CN, —SH, —OCH₃, —OCH₂CH₃, —C(O)CH₃, —NHCH₃, —NHCH₂CH₃, —N(CH₃)₂, —C(O)NH₂, —C(O)NHCH₃, —C(O)N(CH₃)₂, —OC(O)CH₃, —NHC(O)CH₃, —S(O)₂OH, or —S(O)₂NH₂.

A “repeat unit” is the simplest structural entity of certain materials, for example, frameworks and/or polymers, whether organic, inorganic or metal-organic. In the case of a polymer chain, repeat units are linked together successively along the chain, like the beads of a necklace. For example, in polyethylene, -[—CH₂CH₂-]ₙ—, the repeat unit is —CH₂CH₂—. The subscript “n” denotes the degree of polymerization, that is, the number of repeat units linked together. When the value for “n” is left undefined or where “n” is absent, it simply designates repetition of the formula within the brackets as well as the polymeric nature of the material. The concept of a repeat unit applies equally to where the connectivity between the repeat units extends three dimensionally, such as in metal organic frameworks, modified polymers, thermosetting polymers, etc.

### B. PRO-NANOPARTICLE FORMULATIONS AND USES THEREFOR

The inventor developed novel in situ self-assembly nanoparticles (ISNPs) to address multiple hurdles of application of nanotechnology as well as explore the ISNP nanotechnology for various administration routes.

The ISNP Nanotechnology is not Only Scientifically Novel, but Also Behaviorally Superior to Other Existing Technologies.

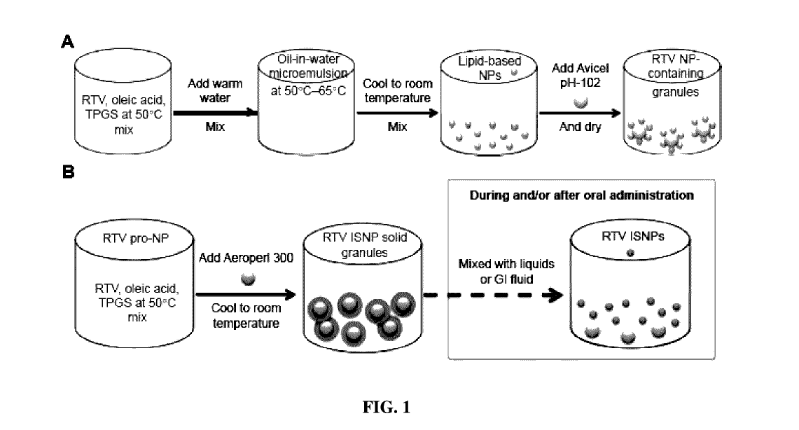

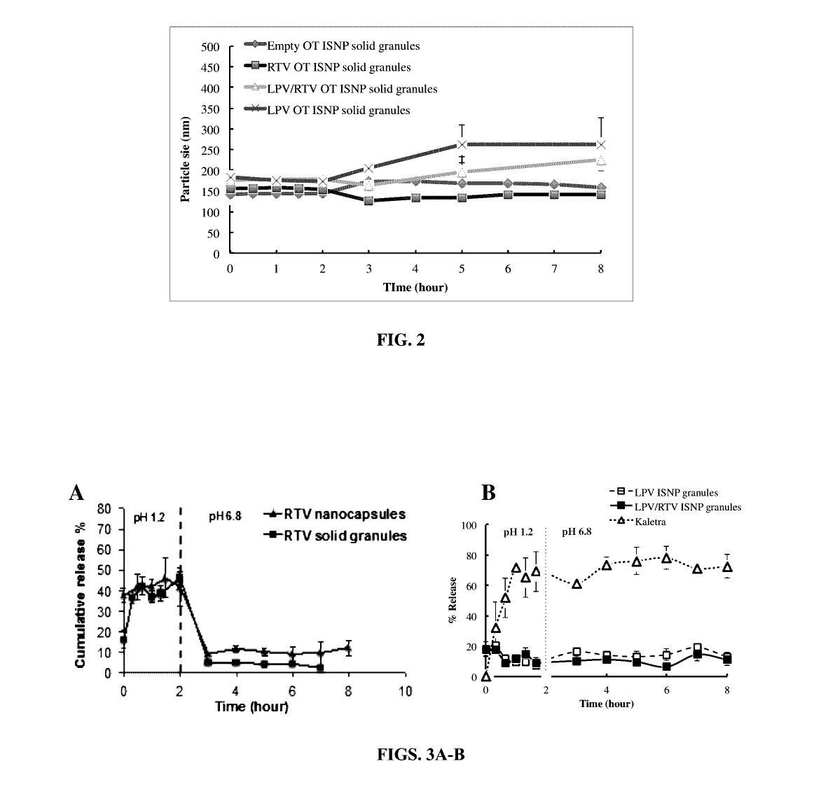

Conventional nanoparticles (NPs) and microemulsions are generally prepared in liquids, leading to instability at long-term storage (Das and Chaudhury 2011, Khan et al., 2012). Research efforts were directed toward converting liquid formulations into solid forms; however, drug loading was very low (i.e., 0.35% (Mahmoud et al., 2009) and 1.7% (Taha et al., 2009) or 0.2 mg drug/tablet (Li et al., 2011)). By completely removing water from the procedure, the inventor prepared drug-loaded “pro-NP (like a pro-drug)”. Then, they converted the pro-NPs to solid ISNP granules by mixing them with a solid carrier. Interestingly, both pro-NPs and granules do not contain NPs; however, when they are in contact with water, the materials release and spontaneously form drug-loaded ISNPs (FIG. 1B). By comparing with RTV NP-containing solid granules prepared by the conversion of nanocapsules (FIG. 1A), the inventor demonstrated that RTV ISNP granules dramatically increased the drug loading over 16 fold. The ISNP nanotechnology changes the concept of preparation of nanoparticle-containing solid dosage forms. The ISNPs are formed after or during administration, not during formulation manufacturing (FIG. 1B). Second, the ISNPs are unique and different from self-microemulsifying and self-nanoemulsifying drug delivery systems (SMEDDS and SNEDDS) that produce micro- or nano-emulsions upon mild agitation in aqueous media. The pilot data in this application manifest the difference between the ISNP nanotechnology and solid self-microemulsifying drug delivery systems. In addition to compositions and particle size, our ISNPs are fundamentally different from microemulsion. The ISNPs are stable NPs, whereas microemulsions are thermodynamically stable under a particular range of conditions (Khan et al., 2012). If the environmental conditions are altered into a range where microemulsions are no longer thermodynamically stable, they may breakdown. For example, dilution, pH change, temperature change and mixing with other surfactants may breakdown microemulsions. Very few publications in this area characterized drug-loaded NPs and reconstitution properties of solid SMEDDS and SNEDDS. In contrast, the inventor's ISNPs were very stable against dilution and reconstitution. When the inventor prepared drug-loaded pro-ISNPs for characterization, the granules were reconstituted over 2000 times in water. With this extremely high dilution, monodispersed ISNPs were still detected with particle size about 170 nm and over 95% of EE % for both LPV and RTV (Table 1). The ISNPs were physically stable in pH 1.2 for 2 hours and in pH 6.8 for 6 hours at 37° C. (FIG. 2). In addition, LPV and RTV slowly released from the ISNPs for 8 hours in simulated physiological conditions (FIG. 3). Moreover, when the granules were reconstituted with 28% poloxamer 188, the same ISNPs as reconstituted with water were detected. Without wishing to be bound by any theory, it is believed that the ISNPs are nanocapsules. Finally, unlike wet granulation and melt granulation, drugs are entrapped in the formed ISNPs after the ISNP granules mix with water. Taken together, this study represents a novel ISNP nanotechnology that is different from previously described methods. To the best of the inventor's knowledge, this is the first report on such nanotechnology to prepare ISNPs.

1. Nanoparticle Formulations

Biopharmaceutics Classification System (BCS) classifies oral drugs into: BCS I—high permeability and high solubility, BCS II—low solubility and high permeability, BCS III—high solubility and low permeability, and BCS IV—low solubility and low permeability. Low bioavailability is often associated with BCS II, III and IV drugs. The drug discovery approaches have a tendency to discover poor soluble and/or poor permeable drug candidates, leading to high demands on improving drug bioavailability through formulation development (Lipinski 2000). Most formulation technologies focus on improved solubility for BCS II drugs. Very few technologies are available to solve permeability issues for BCS III and BCS IV drugs. The lack of effective delivery systems for these drugs may lead to abandoning the development of oral formulations of molecules, nevertheless having strong therapeutic potentials. In addition to new developing drug candidates, over 40% of essential medicines on the World Health Organization (WHO) model list are BCS III drugs (Lindenberg et al., 2004). Many BCS III drugs are associated with low bioavailability, high inter- and intra-subject variability and food effects. Fixed-dose combinations (FDCs) make it possible to combine two or more drugs with different modes of pharmacological actions in a single dosing unit. The main challenge on developing FDCs usually involves product formulation and manufacturing issues. When the drugs have different properties, a formulation technology is required to improve both solubility and permeability. In addition, the formulation technology also needs to mask the taste of drugs for pediatric FDCs. Oral sustained release (SR) drug delivery systems can maintain drug level in the target tissue by controlling the rate of drug delivery to the target tissue and consequently allow a reduction in dosing frequency. Common SR oral delivery systems include reservoir systems, osmatic pumps, matrix systems and polymer-coated multiparticulates such as beads, granules, and microspheres. The limitations of these technologies include high risks on dose dumping after administration, very long processes for manufacturing, limited drug loading on beads, and complicated and time-consuming development. Finally, over 240 oral cytotoxic drugs are using in children without pediatric formulations. Crushing tablets and opening capsules are very common for these drugs. In addition to the risks of unlicensed use, unsafe exposure of parents and children to these cytotoxic drugs is problematic. The inventor has utilized the ISNP nanotechnology to develop solid dosage formulations for poorly water-soluble drugs and poorly permeable drugs. These compositions circumvent a number of the problems and challenges on oral solid dosage forms mentioned above.

For the infections diseases (i.e., HIV) that need chronic treatment, achieving compliance with medication intake plays a crucial role. Indeed, non-compliance has been identified as a critical risk factor for resistance development, further underlining the importance of improving adherence. Long-acting formulations of medications that need only infrequent dosing could facilitate maintenance therapy. There are few technologies available to develop long-acting injections. The nanotechnology in this disclosure provides an alternative for development of long-acting injections.

For example, many anti-cancer drugs are poorly water-soluble drugs, such as docetaxel and curcumin. Developing injections for these drugs are difficult. Often, high amount of solvents and surfactants have to be used to dissolve the drugs, causing severe side effects attributed to the excipients in the injections. NPs can formulate poorly water-soluble drugs and target to tumors. However, NPs are prepared in the present of water that will increase degradation of drugs and consequently result in stability issues for long-term storage. The technology in this disclosure permits complete removal of water from NP preparations, and the nanoformulations are reconstitutable with saline to provide injections.

Many treatments for local skin diseases are relying on systemic administration because of the limited permeability of skin. For example, systemic treatment for acne includes oral antibiotics, retinoids and hormonal treatment. Drug resistance could be a major issue for the treatments. In spite of various available topical treatments, conventionally available dosage forms/delivery systems usually produce a high incidence of side effects, such as skin dryness, peeling and skin irritation. The nanotechnology in this disclosure provides a novel carrier-based drug delivery system to reduce these side effects in order to achieve therapeutic efficacy by local treatments.

2. Administration

Lack of pediatric formulations has led to the need to break tablets or open capsules for administration, risking reduced efficacy and adverse effects because of inaccurate dosing. The human immunodeficiency virus (HIV) infection continues to have devastating effects on human health and the global economy (Durand and Flexner, 2013). In June 2013, the WHO released new guidelines for combination antiretroviral therapy (cART) (World Health Organization, 2013). With the impact, a total of 9.5 million children will become eligible for ART, significantly raising the demand for antiretroviral drugs for infants and children (Nelson et al., 2013). HIV treatment of infants and children is severely limited by commercially available pediatric formulations. Indeed, poor taste, need of cool-chain for storage and transportation, poor adherence, cost, and high pill burdens are all limitations of currently available pediatric antiretroviral formulations. The US guidelines recommend that one complete cART for HIV-infected children comprise two-nucleoside reverse transcriptase inhibitors (NRTI) plus one non-nucleoside reverse transcriptase inhibitors, or a protease inhibitor (PI) such as ritonavir (RTV)-boosted lopinavir (LPV) or atazanavir (ATV) (National Institutions of Health 2013). Single-unit regimen, in which patients take one unit of formulation to finish one complete cART, has been demonstrated to improve patient adherence and virologic response. To date, only four Food and Drug Administration (FDA)-approved single-unit regimens are available, including Atripla, Complera, Triumeq and Stribild. However, none of them includes PIs, likely due to poor water solubility of PIs. Moreover, there are no pediatric formulations for single-unit regimens.

The ideal pediatric formulations should be flexible and adaptable to dose adjustment, palatable, stable, affordable, safe and effective (Walsh and Mills, 2013). Challenges on oral liquid formulations include taste-masking agents, preservatives, excipients, and solubility and stability limitations (World Health Organization, 2012; Walsh 2012; Tuleu and Breitkreutz, 2013). Thus, the WHO promotes flexible solid oral dosage forms as optimal agents for pediatric administration; these include sachets to mix with liquids, sprinkles to pour over food, and oral disintegrating tablets (ODTs) (World Health Organization, 2011). These products can be manufactured using conventional formulation facilities, resulting in more affordable forms than liquids. However, this presents more challenges for water-insoluble drugs than for water-soluble drugs.

Drug bioavailability of current PIs can be reduced by pre-systemic metabolism by the enzyme CYP3A4. RTV is therefore used as a pharmacokinetic (PK) enhancer to “boost” the plasma concentrations of co-administered PIs by inhibiting CYP3A4 (Antonelli al., 2012). Except for darunavir (DRV), the PIs are structurally related peptidomimetic compounds, possessing moderate molecular size, high intrinsic lipophilicity, and pH-dependent solubility. These unfavorable physico-chemical properties compromise the development of suitable pediatric formulations. For example, both commercial LPV/RTV and RTV solutions, contain high concentrations of propylene glycol (>15.3%) and ethanol (>42.4%), making them highly unpalatable and requiring refrigeration. Multiple drugs have to be taken separately by HIV-infected children for effective viral suppression with cART. LPV/RTV oral solution is the only combination of PIs available for infant and children. LPV/RTV should be taken with two NRTIs, e.g. lamivudine (3TC) and zidovudine (AZT), twice daily. For children weighing <25 kg, 4 to 5 packets of the commercial ATV powder have to be taken with the RTV oral solution and two NRTIs, e.g. emtricitabine (FTC) and tenofovir disoproxil fumarate (TDF), once daily with food. Pill/drug burden and unpalatability greatly influence patient adherence—setting children up for suboptimal drug exposure, virologic failure and the development of drug resistance. In addition, the need of a cold chain for storage and transportation increases drug costs and limits antiretroviral options in resource-limited regions. These challenges compromise the benefits of cART in HIV-infected infants and children compared to HIV-infected adults. Pediatric formulations for antiretroviral drugs are listed on the National Institutions of Health Best Pharmaceuticals of Children Act and also on the list of the WHO Missing Drugs and Formulations for Pediatric HIV Treatment. Therefore, developing appropriate pediatric combinations to complete one regimen will greatly improve the efficacy of cART in children.

Lipid NPs have a great potential to improve oral bioavailability of poor water-soluble drugs by presenting the drug in a solubilized state in colloidal dispersion. The excipients in these systems are easily taken up by the body, and the solubility of the encapsulated drug in NPs will not be affected by the gastrointestinal (GI) environment, and food effects will be minimized. Encapsulating drugs into NPs could also mask taste of drugs. Thus, lipid NPs are excellent formulation approaches for poorly water-soluble drugs; however, they are prepared as liquids, leading to stability, handling and manufacturing difficulties. Lack of a suitable process that converts lipid NPs into solid dosage forms limits the applications of lipid NPs.

The inventor developed novel NPs designated as the OT NPs composed of oleic acid and vitamin E TPGS, and the MT NPs composed of Miglyol® 812 and vitamin E TPGS. Both were formed using an emulsion method. The inventor recently discovered that they do not need to use water in the preparation of the OT NPs and the MT NPs. With this change, they developed pro-in situ self-assembly nanoparticle (pro-ISNP) compositions. These pro-ISNP compositions are prepared by mixing the lipids, the surfactants and the therapeutic agents in the absence of water. When the pro-ISNP compositions are reconstituted into a liquid under gentle shaking (e.g. water, juice, the fluid in GI tract or saline for injection reconstitution), the ISNPs are formed. In this nanotechnology, the inventor completely removed water from the preparation procedure, and thus are able to use this ISNP nanotechnology for multiple applications on formulation development which may have limited stability in aqueous environment. See Example 1.

Preparing NPs by in situ self-assembly is a novel method. By completely removing water from NP preparation, the inventor can increase drug loading in the formulation, enhance stability for long-term storage, and manufacture solid dosage forms that produce NPs in contact with water or gastrointestinal fluid with gentle agitation. The manufacturing process of the ISNPs is simple and scalable. It can be applied for any water-insoluble drugs, and may also be used for combinations of water-soluble drugs with water-insoluble drugs, e.g., for HIV combination therapy. Improving drug taste by encapsulating drugs inside NPs is also a new approach and a novel application of nanotechnology. Finally, using sustained release (SR) NPs in solid dosage forms is a new way to prepare SR formulations and apply nanotechnology to water-insoluble drugs for SR formulations. The inventor developed pro-ISNPs and mix them with adsorption agents to form solid ISNP granules or ODTs, which are reconstitutable into syrups. Using this design, the final formulations will be heat-stable and palatable. In one manufacturing process, the inventor will produce flexible oral dosage forms, particularly for use with infants and children. This manufacturing process of ISNP solid granules is simple and scalable.

i. Oral Administration

Flexible Oral Solid Dosage Forms and Fixed-Dose Combinations.

The inventor easily converted drug-loaded ISNPs to solid granules by adding adsorption. Pilot data demonstrate that these formulations were stable, comparable with original nanoparticles, and improved bioavailability compared to the controls. Importantly, because of completely removing water from the preparation, solid granules with high drug loadings (>16%) can be manufactured. The novel ISNP nanotechnology platform can formulate poorly water-soluble drugs and improve bioavailability to provide appropriate formulations, especially for pediatric and geriatric patients. Three HIV protease inhibitors including RTV, LPV and ATV, and two NRTIs including FTC and TDF have been selected as the model drugs to demonstrate the platform using the OT ISNP. The ISNP granules improved the taste of the drugs. The ISNP solid granules have good flow properties, so that they can be compressed into ODTs by direct compression. Moreover, one can add any drugs (water soluble or water-insoluble) into the ISNPs or mix with the ISNPs to develop the FDC of drugs. For example, the inventor has successful developed LPV/RTV ISNP granules and ATV/RTV/FTC/TDF solid ISNP granules containing 4 HIV drugs to complete a regimen, which will greatly reduce pill burdens. Next, the inventor will develop (1) ATV/RTV ISNP granules and ODTs to reduce food effect, (2) ATV/RTV/FTC/TDF FDC ODTs, (3) LPV/RTV/3TC/AZT ISNP granules and FDC ODTs, (4) novel ISNP compositions to enhance taste masking, and (5) novel ISNP granules and ODTs for oral anti-cancer drugs. Flexible solid dosage forms can be reconstituted to suspensions and given to patients unable to swallow tablets or capsules, such as pediatric and geriatric patients. The novel platform can be applied for any oral formulations to overcome the challenges in oral drug formulation and delivery. See Example 2, Example 3 and Example 4.

Oral SR ODTs.

Novel sustained-release technologies are needed to formulate BCS II and BCS IV drugs, and also the drugs that encounter the first pass effects. Lipid-based nanoparticles are excellent delivery systems for poorly water-soluble drugs. Lipid nanoparticles are expected to control drug release. Often, drugs are quickly released from the nanoparticles at the initial stage and then slowly release for a prolonged time. These kinds of release profiles are optimal for drug pharmacokinetics by giving an initial drug concentration to quickly achieve the therapeutic concentration and then keeping an adequate concentration to maintain the treatment over time. According to this disclosure, one can prepare sustained-release nanoparticles by optimizing nanoparticle compositions. Then, one can convert the sustained-release nanoparticles to solid dosage forms. Using sustained-release NPs in solid oral dosage forms is a new approach to the preparation of sustained-release formulations and apply nanotechnology to water-insoluble drugs for SR formulations.

ii. Parenteral Administration

Long-Acting Injections.

Because of completely removing water from nanoparticle preparation, appropriate stability is provided to ensure the use of the ISNPs for injection. The ISNPs are reconstitutable with saline or other injectable solutions or solvents. Instead of oral administration, the inventor subcutaneously injected the reconstituted LPV/RTV OT ISNPs into rats to test the feasibility for long-acting injectable formulation. The inventor will modify the LPV/RTV OT ISNPs to provide a long acting injection to dose for once a week. See Example 5.

Injections to Treat Cancers, Especially Drug Resistant Cancers.

The inventor developed docetaxel (DTX) MT NPs to treat metastatic castration-resistant prostate cancer. Prostate cancer (PCa) is the second most common cause of cancer-related mortality in men in the United States. Androgen deprivation treatment is effective for advanced PCa. However, most of advanced PCa become resistant to deprivation treatment and progresses to castration-resistant prostate cancer (CRPC), and further to an incurable metastatic stage (mCRPC). Docetaxel (DTX)-based chemotherapy is the standard of care for mCRPC; however, eventually patients develop DTX resistance. Predominantly, the cancer-related mortality stems from the development of DTX resistance.

To overcome DTX-resistant mCRPC, the inventor developed DTX-loaded MT NPs by using an emulsion method. Recently, the inventor found that the MT NPs also can be prepared with the inventor's novel ISNP nanotechnology. The inventor can make DTX MT ISNPs using the ISNP nanotechnology to get DTX MT pro-ISNP. Once adding saline to reconstitute the DTX MT pre-ISNP, the inventor will obtain DTX ISNP suspensions that can be used as injections. See Example 6. The inventor also developed curcumin (CUR) MT NPs. Similar with DTX MT NPs, the inventor can use the ISNP nanotechnology to prepare CUR MT pro-ISNPs that kept the properties of the CUR MT NPs. To the best of the inventor's knowledge, they are the first to develop CUR nanoformulations as therapeutic agents to treat mCRPC. See Example 7.

iii. Topical Administration

The inventor envisions that the nanoparticle formulations of the present application may advantageously be employed through topical administration. Generally, topical drug products are those administered to the skin or another external body surface such as a mucous membrane. Well-known examples include lotions, foams, ointments, cremes, gels and pastes. The ISNPs can be mixed with based or gelling agents to form NP-based topical formulations to enhance skin permeability. Moreover, oleic acid is known to have anti-inflammatory effect, and TPGS is known to improve skin penetration. Thus, the OT NPs will have great potential as a carrier-based delivery system for skin diseases. See Example 8.

A transdermal drug product is another type of topical drug, but instead of treating the skin itself, it treats a condition by providing the drug systemically. In addition to various forms of transdermal patches, ointment, cream, solution, gel and foam formats are commonly used for transdermal formulations.

Another form of topical agent is a cosmetic, which generally defined cleanses, beautifies, promotes attractiveness, or alters appearance without affecting the body's structure or functions.

Beauty care or cosmetic formulations may comprise 40 or more ingredients, and may be heterogeneous, with complex microstructures. A ‘cosmeceutical’ is nominally a cosmetic-pharmaceutical hybrid, a cosmetic product that comprises one or more biologically active agents. Similar to cosmetics are topical personal care products include those for exterior personal cleanliness and hygiene.

Also, skin-applied medical devices constitute topical agent, and can include wound care dressings (bandages, gauze, sutures, etc.) or protective devices embodying a nanoparticle according to the present disclosure.

### C. PRO-NANOPARTICLE CONSTITUENTS AND METHODS OF SYNTHESIS

1. Pharmaceutically Acceptable Oil Phase

In some aspects, the nanoparticle constituents include one or more oils wherein the oil is a lipid, a polymer, or a mixture thereof. It is envisioned that various oil phases may be used to prepare the pro-nanoparticle compositions of the present disclosure. Some criteria for suitable pharmaceutically acceptable oil phases are oil phases that are (1) soluble in the selected surfactant; (2) either immiscible with water, insoluble in water, or poorly-water soluble; and (3) biocompatible.

In some aspects, the pharmaceutically acceptable oil phase of the present disclosure includes one or more lipids wherein the lipid is a saturated or unsaturated fatty acid. The fatty acids that may be used herein include C₆-C₃₀ alkyl or alkenyl groups which may be substituted with one or more halogen atoms or hydroxyl groups. Some examples of the fatty acids used herein include caprylic acid, capric acid, lauric acid, myristic acid, palmitic acid, stearic acid, arachidic acid, behenic acid, lignoceric acid, cerotic acid, myristoleic acid, palmitoleic acid, sapienic acid, oleic acid, elaidic acid, vaccenic acid, linoleic acid, linoelaidic acid, α-linolenic acid, arachidonic acid, eicosapentaenoic acid, erucic acid, or docosahexaenoic acid.

In various embodiments, an oil phase of the present disclosure may comprise a compound of the structure:

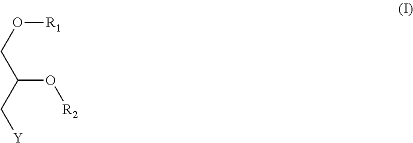

wherein:
R₁ and R₂ are each independently hydrogen or —C(O)—R₄; and
Y is hydrogen, hydroxy, or —OC(O)—R₄; wherein:
   R₄ is alkyl_{(C1-25)}, alkenyl_{(C1-25)}, alkynyl_{(C1-25)}, or a substituted version of any of these groups; or a group of the formula: —C(O)—X—C(O)H, wherein X is an alkanediyl(C₁₋₁₂₎ or substituted alkanediyl(C₁₋₁₂₎;
provided that R₁, R₂, and Y are not all hydrogen.

In some embodiments, R₄ is selected from the group C₁-C₂₅ substituted or unsubstituted alkyl, C₁-C₂₅ substituted or unsubstituted alkenyl, C₁-C₂₅ substituted or unsubstituted alkynyl, and —C(O)—X—C(O)H, wherein X is —(CH₂)_{z}—, wherein Z=1-12. In some embodiments, R₄ is selected from the group C₁-C₂₅ alkyl, C₁-C₂₅ alkenyl, C₁-C₂₅ alkynyl, and —C(O)—X—C(O)H, wherein X is —(CH₂)_{z}—, wherein Z=1-12. In the above structure it is important to note that if one or more of R₁ and R₂ are —C(O)—R₄ and/or Y is —OC(O)—R₄, then a different R₄ group may be associated with R₁, R₂, and/or Y (e.g., R₁, R₂, and/or Y do not need to have the same R₄ group).

In some embodiments, R₁ or R₂ is —C(O)—R₄, wherein R₄ is C₄-C₁₈ alkyl, C₈-C₂₅ alkenyl, or C₈-C₂₅ alkynyl. In other embodiments, R₄ is —(CH₂)_{Y}—H, wherein Y=8-10. In some embodiments, R₁, R₂, and/or R₃ is a caprylic group, a capric group, a linoleic group, or a succinic group. In some aspects, the triglyceride is a composition comprising two or more different triglyceride molecules. The triglyceride composition may further comprise a ratio of the first triglyceride to the second triglyceride from about 4:6 to about 6:4.

As would be appreciated by one of skill, various synthesis reactions may be used to produce a monoglyceride, diglyceride, triglyceride, ester of ethylene glycol or propylene glycol, or diester of ethylene glycol or propylene glycol. For example, an alcohol group present on a glycerol, ethylene glycol, or propylene glycol backbone may be reacted with a carboxylic acid group present on, e.g., caprylic acid, capric acid, linoleic acid, or a dicarboxylic acid such as malonic acid, succinic acid, glutaric acid, adipic acid, pimelic acid, suberic acid, azelaic acid, azelaic acid, or sebacic acid. In some non-limiting examples, the carboxylic acids may be reacted readily with alcohols in the presence of catalytic amounts of strong acids, such as mineral acids, to yield esters (see, e.g., Streitwieser and Heathcock, 1985 among others).

Some non-limiting examples of the pro-ISNP described herein comprise Miglyol™ 812. MIGLYOLs are hydrophobic compositions produced by SASOL Germany GmbH. These compositions are neutral oils of saturated coconut and palm kernel oil-derived caprylic and capric fatty acids and glycerin or propylene glycol. Some examples of useful MIGLYOLs include Miglyol™ 810 and 812 (Caprylic/Capric Triglyceride), Miglyol™ 818 (Caprylic/Capric/Linoleic Triglyceride), Miglyol™ 829 (Caprylic/Capric/Succinic Triglyceride), and Miglyol™ 840 (Propylene Glycol Dicaprylate/Dicaprate).

Other types of oil phases which may be used with the present disclosure include monoglycerides, diglycerides, triglycerides, esters propylene glycol, and diesters or propylene glycol, which may comprise suitable lipophilic groups linked via an ester bond to the glycerol or propylene glycol backbone. Other oil phases which may be used with the present disclosure include but are not limited to: triglyceryl monoleate, glyceryl monostearate, medium chain mono- & diglycerides, glyceryl monocaprate, glyceryl monocaprylate, decaglycerol decaoleate, triglycerol monooleate, triglycerol monostearate, polyglycerol ester of mixed fatty acids, hexaglycerol dioleate, decaglycerol mono- or dioleate, propylene glycol dicaprate, propylene glycol dicaprylate/dicaprate, glyceryl tricaprylate/caprate, glyceryl tricaprylate/caprate/laurate, glyceryl tricaprylate/caprate, triacetin, propylene glycol di-(2-ethylhexanoate), glyceryl tricaprylate/caprate/linoleate, glyceryl tricaprate, glyceryl tricaprylate, glyceryl triundecanoate.

In other aspects, the pharmaceutically acceptable oil phase may also comprise a naturally derived liquid oil such as corn oil, coconut oil, sunflower seed oil, vegetable oil, cottonseed oil, mineral oil, peanut oil, sesame oil, soybean oil, and/or olive oil. Other pharmaceutically acceptable oil phases that may be used with the present disclosure including liquid fatty alcohols, liquid fatty acids, liquid fatty esters, and phospholipids.

In other aspects, the present disclosure provides a pharmaceutically acceptable oil phase which comprises one or more pharmaceutically acceptable polymer. These pharmaceutically acceptable polymers may also include homopolymers, co-polymers, or blends of two or more polymers. Some non-limiting examples of pharmaceutically acceptable polymers include polylactic acid, poly-amino acids, poly(methyl methacrylate), poly(ethyl methacrylate), poly(butyl methacrylate), poly(isobutyl methacrylate), poly(hexyl methacrylate), poly(isodecyl methacrylate), poly(lauryl methacrylate), poly(phenyl methacrylate), poly(methyl acrylate), poly(isopropyl acrylate), poly(isobutyl acrylate), poly(octadecyl acrylate) polyethylene, polypropylene, poly(ethylene glycol), poly(ethylene oxide), poly(ethylene terephthalate), poly(vinyl acetate), polyvinyl chloride, polystyrene, polyvinyl pyrrolidone, polyvinylphenol, polylactides, polyglycolides and copolymers thereof, poly(ethylene terephthalate), poly(butic acid), poly(valeric acid), poly(lactide-co-caprolactone), poly lactide-co-glycolide, polyanhydrides, polyorthoesters, polyphosphazines, poly(vinyl alcohols), polyamides, polycarbonates, polyalkylenes, polyacrylamides, polyalkylene glycols, polyalkylene oxides, polyalkylene terephthalates, polyvinyl ethers, polyvinyl esters, polyvinyl halides, polyvinylpyrrolidone, polyglycolides, polysiloxanes, polyurethanes, blends and copolymers thereof. In some embodiments, the polymer is a polymer which undergoes complete degradation under biological conditions. Such degradation conditions can include pH, temperature, or the activity of proteins such as proteases and may result in greater than 50% degradation, greater than 75% degradation, greater than 80% degradation, greater than 90% degradation, or greater than 95% degradation. In some embodiments, the polymer is a biological polymer such as polylactic acid, poly-hydroxyalkanoates such as poly-3-hydroxybutyrate, polyhydroxyvalerate, or polyhydroxyhexanoate, or polypolyamide such as polyamide 11.

2. Surfactant

In some aspects, the present disclosure provides compositions comprising one or more surfactants. As used herein, a “surfactant” refers to a surface-active agent, including substances commonly referred to as wetting agents, detergents, dispersing agents, or emulsifying agents. In some embodiments, the surfactant has an HLB value of about 6-20. Methods of calculating HLB are known in the literature. The surfactant may be either non-ionic, ionic, or cationic, and may be a polyoxyethylene alkyl ethers, polyethylene glycol and polypropylene glycol co-polymer, polyethylene glycol vitamin derivative, polyoxyethylene sorbitan fatty acid esters, phospholipids, polyoxyethylene stearates, fatty alcohols or their derivatives, hexadecyltrimethylammonium bromide, or combinations thereof. In some embodiments, the surfactant used in the present disclosure is be chemically modified with a molecule (e.g., polyethylene glycol or polyoxyethylene) to promote increased circulation durations in the blood. Additionally, it is envisioned that the surfactants could be chemically modified with a cell-targeting ligand such as a small molecule, peptide, protein, or carbohydrate. Surfactants of the present disclosure are pharmaceutically acceptable surfactants which result in little or no toxicity when administered to a subject according to the present disclosure. In some embodiments, the surfactant is a cell-targeting ligand which has been modified with one or more other chemicals groups such as a polyethylene glycol group or a polypropylene glycol group.

In some embodiments, the compositions may further comprise a “co-surfactant”. The term “co-surfactant” refers to a surface-active agent, including substances commonly referred to as wetting agents, detergents, dispersing agents, or emulsifying agents. In some embodiments, the co-surfactant is polyoxyethylene alkyl ethers, polyoxyethylene sorbitan fatty acid esters, polyoxyethylene stearates, or fatty alcohols or their derivatives, hexadecyltrimethylammonium bromide, or combinations thereof.

In some embodiments, the compositions of the present disclosure comprise a surfactant wherein the surfactant is a modified small molecule. In some embodiments, the modified small molecule is modified by the addition of a polyethylene glycol or a polypropylene glycol group. In some embodiments, the surfactant is a PEGylated tocopheryl succinate compound. In some embodiments, the PEGylated tocopheryl succinate comprises a tocopheryl succinate of a formula:

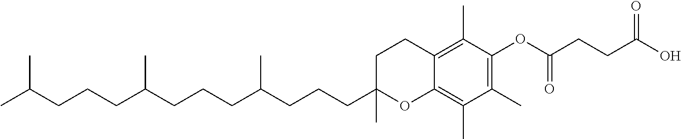 [SMILES: Cc1c(C)c2c(c(C)c1OC(=O)CCC(=O)O)CCC(C)(CCCC(C)CCCC(C)CCCC(C)C)O2]

and a PEGylated group attached to the free carboxyl group. In some embodiments, the PEG group comprises a repeating unit of ethylene glycol with a number of repeating units from 1 to 1,000. PEG is the polymeric form of ethylene glycol. The PEG portion of the compound has the formula:

Tocophery-(OCH₂CH₂)ₙOH (I)

wherein the repeating unit, n, is an integer. The number of repeating units may be from about 1, 5, 10, 15, 20, 25, 50, 75, 100, 200, 300, 400, 500, 600, 700, 800, 900, to about 1,000 units, or any range derivable therein. In some aspects, the nomenclature used to describe PEG includes the average molecular weight of the polymer (e.g. PEG-800; PEG-1000, PEG-1200, etc.). As would be obvious to a person of skill in the art, the average molecular weight does not mean that any particular PEG component within the composition has the noted molecular weight but rather that the component as a whole has the average molecular weight corresponding to that value. In some embodiments, the PEG component can have a terminal hydrogen atom can be replaced with another group including but not limited to a C₁-C₆ alkyl group (e.g. a methyl group or an ethyl group), or a reactive moiety used to attach the PEG to another compound. For example, a PEG-1000 composition generally comprises PEG molecules with 16 and 17 repeating units as shown in the formula above, but may also comprises individual PEG molecules with less than 16 or more than 17 repeating units. As the value in the name of the PEG component represents the average molecular weight, the overall polymer average molecular weight may be modified to obtain an average molecular weight from less than 500 to over a 2500 g/mol (e.g. about 10 repeating units to about 40 repeating units). In some embodiments, the PEG component of the molecule has an average molecular weight equal to or less than PEG-1000.

3. Absorption Agent

In some aspects, the pro-nanoparticle compositions may further comprise an absorption agent. The absorption agent is a compound which absorbs the solution to form a composition which contains the pro-nanoparticles and has a solid form which can be administered to a patient. In some embodiments, the absorption agent is pharmaceutically acceptable and capable of being administered to a patient. The absorption agent is an excipient which is capable of formulating the compound into an oral dosing form. In some aspects, the absorption agent is a metal oxide such as silica, alumina, titania, or a mixture of any of these compounds. In some embodiments, the absorption agent is a silica. In other embodiments, the absorption agent is a cellulosic polymer. In some embodiments, the absorption agent is microcrystalline cellulose. In other aspects, the absorption agent is an excipient such as a sugar, polysaccharide, or amino acid. In some embodiments, the excipient is a sugar molecule or sugar derivative such as mannitol or lactose. In other embodiments, the excipient is a polysaccharide such as starch.

4. Bases and Gelling Agents

In some aspects, the pro-nanoparticles may be reconstituted with liquids and mixed with a base or a gelling agent, or directly mixed with a base or a gelling agent, to form a composition which contains the nanoparticles and has a semisolid form which can be administered to a patient. In some embodiments, the base or gelling agent is pharmaceutically acceptable and capable of being administered to a patient. The base or gelling agent is an excipient which is capable of formulating the compound into a topical or transdermal form. In some aspects, the base is an ointment base, such as hydrocarbon bases, absorption bases, water-removable bases, or water-soluble bases. In some embodiments, the gelling agent is a synthetic macromolecule, such as carbomer 934 and carbopols; cellulose derivatives, such as carboxymethylcellulos or hydroxypropyl methylcellulose; and natural gums, such as tragacanth.

5. Synthesis

In some aspects, the compositions of the present disclosure may be prepared by mixing the pharmaceutically acceptable oil phase with the surfactant with each other followed by the addition and mixing of the therapeutic agent to form a pro-ISNP composition. In some embodiments, this mixture is heated to an elevated temperature sufficient to ensure the solubility of all of the components. In some embodiments, this temperature is from about 30° C. to about 100° C. In some embodiments, the temperature is about 30° C., 40° C., 50° C., 55° C., 60° C., 65° C., 70° C., 80° C., 90° C., or 100° C. To this pro-ISNP composition, water, saline, or other injectable pharmaceutically acceptable solutions may be added to the pro-ISNP composition to obtain the in situ nanoparticle compositions. In some embodiments, the water is warmed to the temperature of the pro-ISNP composition. When the water is added to the pro-ISNP composition, the water is added with agitation and/or mixing. When the liquid component (e.g. water, saline, or other injectable pharmaceutically acceptable solutions) is added to the pro-ISNP composition, a nanoparticle suspension is formed. To the pro-ISNP composition, an absorption agent may be added. The absorption agent may be used as an additive to the composition so that it can be formulated as a solid for oral administration. Saline and other injectable solutions may be used to reconstitute the pro-ISNP composition for injection. Water and juice may be added to reconstitute the solid granules to generate a suspension which may then be administered orally.

### D. THERAPEUTIC AGENTS

1. Nucleic Acids

In some aspects of the present disclosure, the nanoparticle compositions comprise one or more nucleic acids. In addition, it should be clear that the present disclosure is not limited to the specific nucleic acids disclosed herein. Formulations of pro-ISNP compositions may further comprise a nucleic acid based therapeutic agents. The present disclosure is not limited in scope to any particular source, sequence, or type of nucleic acid, however, as one of ordinary skill in the art could readily identify related homologs in various other sources of the nucleic acid including nucleic acids from non-human species (e.g., mouse, rat, rabbit, dog, monkey, gibbon, chimp, ape, baboon, cow, pig, horse, sheep, cat and other species). It is contemplated that the nucleic acid used in the present disclosure can comprises a sequence based upon a naturally-occurring sequence. Allowing for the degeneracy of the genetic code, sequences that have at least about 50%, usually at least about 60%, more usually about 70%, most usually about 80%, preferably at least about 90% and most preferably about 95% of nucleotides that are identical to the nucleotide sequence of the naturally-occurring sequence. In another embodiment, the nucleic acid is a complementary sequence to a naturally occurring sequence, or complementary to 75%, 80%, 85%, 90%, 95% and 100%.

In some aspects, the nucleic acid is a sequence which silences, is complimentary to, or replaces another sequence present in vivo. Sequences of 17 bases in length should occur only once in the human genome and, therefore, suffice to specify a unique target sequence. Although shorter oligomers are easier to make and increase in vivo accessibility, numerous other factors are involved in determining the specificity of hybridization. Both binding affinity and sequence specificity of an oligonucleotide to its complementary target increases with increasing length. It is contemplated that exemplary oligonucleotides of 8, 9, 10, 11, 12, 13, 14, 15, 16, 17, 18, 19, 20, 25, 30, 35, 40, 45, 50, 55, 60, 65, 70, 75, 80, 85, 90, 95, 100 or more base pairs will be used, although others are contemplated. Longer polynucleotides encoding 250, 500, 1000, 1212, 1500, 2000, 2500, 3000 or longer are contemplated as well.

As stated above, “complementary” or “antisense” means polynucleotide sequences that are substantially complementary over their entire length and have very few base mismatches. For example, sequences of fifteen bases in length may be termed complementary when they have complementary nucleotides at thirteen or fourteen positions. Naturally, sequences which are completely complementary will be sequences which are entirely complementary throughout their entire length and have no base mismatches. Other sequences with lower degrees of homology also are contemplated. For example, an antisense construct which has limited regions of high homology, but also contains a non-homologous region (e.g., ribozyme; see below) could be designed. These molecules, though having less than 50% homology, would bind to target sequences under appropriate conditions.

Inhibitory RNA.

As mentioned above, the present disclosure contemplates the use of one or more inhibitory nucleic acid for reducing expression and/or activation of a gene or gene product. Examples of an inhibitory nucleic acid include but are not limited to molecules targeted to an nucleic acid sequence, such as an siRNA (small interfering RNA), short hairpin RNA (shRNA), double-stranded RNA, an antisense oligonucleotide, a ribozyme and molecules targeted to a gene or gene product such as an aptamer.

An inhibitory nucleic acid may inhibit the transcription of a gene or prevent the translation of the gene transcript in a cell. An inhibitory nucleic acid may be from 16 to 1000 nucleotides long, and in certain embodiments from 18 to 100 nucleotides long.

In some embodiment, an inhibitory nucleic acid is capable of decreasing the expression of a particular genetic product by at least 10%, at least 20%, at least 30%, or at least 40%, at least 50%, at least 60%, or at least 70%, at least 75%, at least 80%, at least 90%, at least 95% or more or any ranges in between the foregoing.

In some embodiments, the nucleic acids of the present disclosure comprise one or more modified nucleosides comprising a modified sugar moiety. Such compounds comprising one or more sugar-modified nucleosides may have desirable properties, such as enhanced nuclease stability or increased binding affinity with a target nucleic acid relative to an oligonucleotide comprising only nucleosides comprising naturally occurring sugar moieties. In some embodiments, modified sugar moieties are substituted sugar moieties. In some embodiments, modified sugar moieties are sugar surrogates. Such sugar surrogates may comprise one or more substitutions corresponding to those of substituted sugar moieties. In some embodiments, nucleosides of the present disclosure comprise one or more unmodified nucleobases. In certain embodiments, nucleosides of the present disclosure comprise one or more modified nucleobases.

2. Peptides and Proteins

The use of peptides and proteins as drugs continues to grow. As with many complex molecules, delivery issues may prevent the effective use of peptide/polypeptide drugs. Thus, the pro-ISNPs of the present disclosure may find use for the delivery of peptide/polypeptide drugs including but not limited to antibodies (Infliximab, Herceptin, Cetuximab, Rituximab), peptide hormones (insulin), clotting factors, anti-cancer peptides (Adalimumab, Alemtuzumab, Bevacizumab, Bortezomib, Cilengitide, Triptorelin pamoate, Leuprolide acetate, Histrelin acetate, Goserelin acetate, Buserelin acetate, Abarelix acetate, Degarelix acetate), cytokines, interferons, interleukins (IL-1, IL-2, etc.), antivirals (Enfuvirtide), growth factors, enzymes (TPA), and a host of others (Teriparatide, Exenatide, Liraglutide, Lanreotide, Pramlintide, Ziconotide, Icatabant, Ecallantide, Tesamorelin, Mifamurtide and Nesiritude).

3. Small Molecules

The overwhelming majority of drugs—antibiotics, antiviral, cancer chemotherapeutics, anti-hypertensives, statins, anti-depressives, and many others—and many others are categorized as “small molecules,” a general term applied to the class of compounds also described as organopharmaeuticals. In some aspects, these drugs or therapeutic agents are compounds which have a molecular weight of less than 2500 g/mol. In some embodiments, the therapeutic agents have a molecular weight from about 100, 150, 200, 250, 300, 350, 400, 450, 500, 550, 600, 650, 700, 750, 800, 900, 1000, 1250, 1500, 1750, 2000, 2250, to about 2500 g/mol. These therapeutic agents may be compounds which have a definitive structural formula and may be present as a neutral molecule or as a salt. In some embodiments, small molecule therapeutic agents are compounds which have a definitive chemical structure and formula which expressed through a specific connectivity of bonds and atoms. In another embodiment, the therapeutic agents used in the methods described herein are small molecule compounds which are not particular soluble in water. Some non-limiting examples of therapeutic agents are BCS classes II and IV compounds or other agents that similarly exhibit poor solubility. The BCS definition describes a compound in which the effective dosing is not soluble in 250 mL of water at a pH from 1-7.5. The USP categories “very slightly soluble” and “insoluble” describe a material that requires 1,000 or more parts of the aqueous liquid to dissolve 1 part solute. As used herein, when a compound is described as poorly soluble, it refers to a compound which has solubility in water of less than 1 mg/mL.

The methods of the present disclosure may be used to prepare nanoparticles using many classes of therapeutic or cosmetic agents including, but not limited to chemotherapeutics, agents for the prevention and treatment of acne, wrinkles or scars, agents for the prevention of restenosis, agents for treating renal disease, agents used for intermittent claudication, agents used in the treatment of hypotension and shock, angiotensin converting enzyme inhibitors, antianginal agents, anti-arrhythmics, anti-hypertensive agents, antiotensin II receptor antagonists, antiplatelet drugs, β-blockers β1 selective, beta blocking agents, botanical products for cardiovascular indications, calcium channel blockers, cardiovascular/diagnostics, central alpha-2 agonists, coronary vasodilators, diuretics and renal tubule inhibitors, neutral endopeptidase/angiotensin converting enzyme inhibitors, peripheral vasodilators, potassium channel openers, anticonvulsants, antiemetics, antinauseants, anti-parkinson agents, antispasticity agents, cerebral stimulants, drugs to treat head trauma, drugs to assist with memory (e.g., to treat alzheimers/senility/dementia), drugs to treat migraine, drugs to treat movement disorders; also included are drugs to treat a disease such as multiple sclerosis, narcolepsy/sleep apnea, stroke, tardive dyskinesia; chronic graft versus host disease, eating disorders, learning disabilities, minimal brain dysfunction, obsessive compulsive disorder, panic, alcoholism, drug abuse, developmental disorders, diabetes, benign prostate disease, sexual dysfunction, rejection of transplanted organs, xerostomia, aids patients with kaposi's syndrome; antineoplastic hormones, biological response modifiers for cancer treatment; also included are vascular agents, cytoxic alkylating agents, cytoxic antimetabolics, cytoxics, immunomodulators, multi-drug resistance modulators, radiosensitizers, anorexigenic agents/CNS stimulants, antianxiety agents/anxiolytics, antidepressants, antipsychotics/schizophrenia, antimanics, sedatives and hypnotics, enkephalin analgesics, hallucinogenic agents, narcotic antagonists/agonists/analgesics, analgesics, epidural and intrathecal anesthetic agents, general, local, regional neuromuscular blocking agents sedatives, preanesthetic adrenal/acth, anabolic steroids, dopamine agonists, growth hormone and analogs, hyperglycemic agents, hypoglycemic agents, large volume parenterals (lvps), lipid-altering agents, nutrients/amino acids, nutritional lvps, obesity drugs (anorectics), somatostatin, thyroid agents, vasopressin, vitamins other than d, antiallergy nasal sprays, antiasthmatic dry powder inhalers, antiasthmatic metered dose inhalers, antiasthmatics (nonsteroidal), (antihistamines, antitussives, decongestants, etc.), beta-2 agonists, bronchoconstrictors, bronchodilators, cough-cold-allergy preparations, inhaled corticosteroids, mucolytic agents, pulmonary anti-inflammatory agents, pulmonary surfactants, anticholinergics, antidiarrheals, antiemetics, cathartics and laxatives, cholelitholytic agents, gastrointestinal motility modifying agents, h2 receptor antagonists, inflammatory bowel disease agents, irritable bowel syndrome agents, liver agents, metal chelators, miscellaneous gastric secretory agents, miscellaneous gi drugs (including hemorrhoidal preparations), pancreatitis agents, pancreatic enzymes, prostaglandins, prostaglandins, gi, proton pump inhibitors, sclerosing agents, sucralfate, anti-progestins, contraceptives, oral contraceptives, estrogens, gonadotropins, gnrh agonists, gnrh antagonists, oxytocics, progestins, uterine-acting agents, anti-anemia drugs, anticoagulants, antifibrinolytics, antiplatelet agents, antithrombin drugs, coagulants, fibrinolytics, hematology, heparin inhibitors (including protamine sulfate & heparinase), blood drugs (e.g., drugs for hemoglobinopathies, hrombocytopenia, and peripheral vascular disease), prostaglandins, vitamin k, anti-androgens, androgens/testosterone, gnrh agonists, gnrh antagonists, aminoglycosides, antibacterial agents, sulfonamides, antibiotics, antigonorrheal agents, anti-resistant antimicrobials, antisepsis immunomodulators, antitumor agents, cephalosporins, clindamycins, dermatologics, detergents, erythromycins, macrolides, anti-infectives (topical), other systemic antimicrobial drugs, otic-antibiotic in combination, penem antibiotics, penicillins, peptides—antibiotic, sulfonamides, systemic antibiotics, immunomodulators, immunostimulatory agents, aminoglycosides, anthelmintic agents, antibacterial (bacterial vaginosis), antibacterial—quinolones, antifungal (candidiasis), antifungal, systemic, anti-infectives/systemic, antimalarials, antimycobacterial, antiparasitic agents, antiprotozoal agents, antitrichomonads, antituberculosis, chronic fatigue syndrome, immunomodulators, immunostimulatory agents, macrolides, other drugs—aids related illnesses, other antiparasitic antimicrobial drugs, spiramycin, systemic antibiotics anti-gout drugs, corticosteroids, systemic, cyclooxygenase inhibitors, enzyme blockers, immunomodulators for rheumatic diseases, metalloproteinase inhibitors, nonsteroidal anti-inflammatory agents, non-steroidal anti-inflammatory agents, antifungals, antihistamines, contraceptives, detergents, non-narcotic analgesics, nsaids, vitamins, analgesics, nonnarcotic, antipyretics, counterirritants, muscle relaxant, anticaries preparations, antigingivitis agents, antiplaque agents, antifibrinolytics, chelating agents, alpha adrenergic agonists/blockers, antibiotics, antifungals, antiprotozoals, antivirals, beta adrenergic blockers, carbonic anhydrase inhibitors, corticosteroids, immune system regulators, mast cell inhibitors, nonsteroidal anti-inflammatory agents, pro staglandins, and proteolytic enzymes.

Some non-limiting examples of therapeutic agents include albendazole, p-aminosalicylic acid, naproxen, carbamazepine, paclitaxel and other taxanes, carvedilol, ticagrelor, phenytoin, rifaximin, nimesulide, domperidone, retinotic acid, benzoyl peroxide, salicylic acid, clindamycin, candesartan cilexetil, telmisartan, antiodarone, felodipine, diazepam, metaxalone, aceclolenae, hydrochlorothiazide, zaleplon, glipizide, repaglinide, glibenclamide, and praziquantel.

One class of small molecule therapeutic agents is natural products. A natural product refers to a compound which origins from a living organism and was first obtained by purification from a mixture of compounds from the living organism. Some non-limiting examples of natural products are compounds which are derived from a plant such as taxol, aspirin, curcumin, or a polyphenol such as resveratrol and epigallocatechin.

In other aspects, the small molecule therapeutic agent is a diagnostic agent. Some non-limiting examples of diagnostic agents include, but are not limited to, magnetic resonance image enhancement agents, positron emission tomography products, radioactive diagnostic agents, radioactive therapeutic agents, radio-opaque contrast agents, radiopharmaceuticals, ultrasound imaging agents, and angiographic diagnostic agents.

### E. KITS

The present disclosure also provides kits. Any of the components disclosed herein may be combined in the form of a kit. In some embodiments, the kits comprise a pro-ISNP or other pro-nanoparticle composition as described above or in the claims.

The kits will generally include at least one vial, test tube, flask, bottle, syringe or other container, into which a component may be placed, and preferably, suitably aliquoted. Where there is more than one component in the kit, the kit also will generally contain a second, third or other additional containers into which the additional components may be separately placed. However, various combinations of components may be comprised in a container. In some embodiments, all of the delivery components are combined in a single container. In other embodiments, some or all of the delivery components with the instant pro-ISNP or other pro-nanoparticle compositions are provided in separate containers.

The kits of the present disclosure also will typically include packaging for containing the various containers in close confinement for commercial sale. Such packaging may include cardboard or injection or blow molded plastic packaging into which the desired containers are retained or a glass vial containing a syringable composition. A kit may also include instructions for employing the kit components. Instructions may include variations that can be implemented.

### F. EXAMPLES

The following examples are included to demonstrate preferred embodiments of the disclosure. It should be appreciated by those of skill in the art that the techniques disclosed in the examples which follow represent techniques discovered by the inventor to function well in the practice of the disclosure, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the disclosure.

### Example 1

### Two Different Methods to Prepare Novel Nanoformulations

An emulsion method was used to prepare the OT and MT NPs:
(1) OT NPs are composed of water, oleic acid and D-alpha-tocopheryl polyethylene glycol 1000 succinate (TPGS). Briefly, 4 mg oleic acid and 8 mg TPGS (1:2, w/w) were added into a glass vial, and heated to 60° C. and mixed for 5 min. Two ml of pre-heated Milliq water were added into the mixture and stirred for 20 min. After cooled down to room temperature, the OT NPs will be formed, which are suspension.
(2) MT NPs are composed of water, Migloyl 812 and TPGS. Briefly, 4 mg Migloyl 812 and 4 mg TPGS (1:1, w/w) were heated to 35° C. Two (2) ml of filtered and Milliq water pre-heated at 65° C. was added into the mixture. The mixture was stirred for 20 min then cool down to room temperature. The MT nanoparticle suspension will be produced.

Properties of MT NPs.

The inventor can lyophilize the MT NP, DTX MT NPs and

CUR MT NPs without losing the properties of the NPs. After lyophilization, they can reconstitute the lyophilized powder to the NPs by adding water and gently shaking. This does not require adding of cryoprotectants for lyophilization. Thus, they can ensure long-term stability of the NPs as parenteral formulations by lyophilization. Also, the NPs can pass 0.2 μm filters without changes. Thus, problems relating to the sterilization of NPs for injections have been addressed.

Novel In Situ Self-Assembly Nanoparticles (ISNP).

This is a novel method to prepare nanoformulations:
(1) OT ISNPs are composed of oleic acid and D-alpha-tocopheryl polyethylene glycol 1000 succinate (TPGS) at a 1:2 ratio (w/w). Briefly, oleic acid and TPGS were added into a glass vial, and mixed for 10 min at 60° C. After cooled down to room temperature, the OT pro-ISNP composition was formed. After adding 1 ml Milliq water with gentle agitation, the inventor obtained OT ISNPs (NP suspension). The inventor measured the NP properties using the NP suspension.
(2) MT ISNPs are composed of Migloyl™ 812 and TPGS at a 1:1 ratio (w/w). The preparation procedure was the same with that for the OT ISNPs.

Preparing ISNP Solid Granules.

The inventor added absorption agents, such as Avicel PH 102 or Aeroperl 300, into the melted excipients. Briefly, oleic acid (or Migloyl™ 812) and TPGS were added into a glass vial at the defined ratio as described above, and mixed for 10-15 min at 60° C. to form pro-ISNP composition. Then, solid absorption agents were added into the pro-ISNP composition at 60° C. and mixed for 1 min to form granules. Then, the inventor let the granules cool down to room temperature with mixing to form the pro-ISNP solid granules.

Drug-Loaded Nanoformulations.

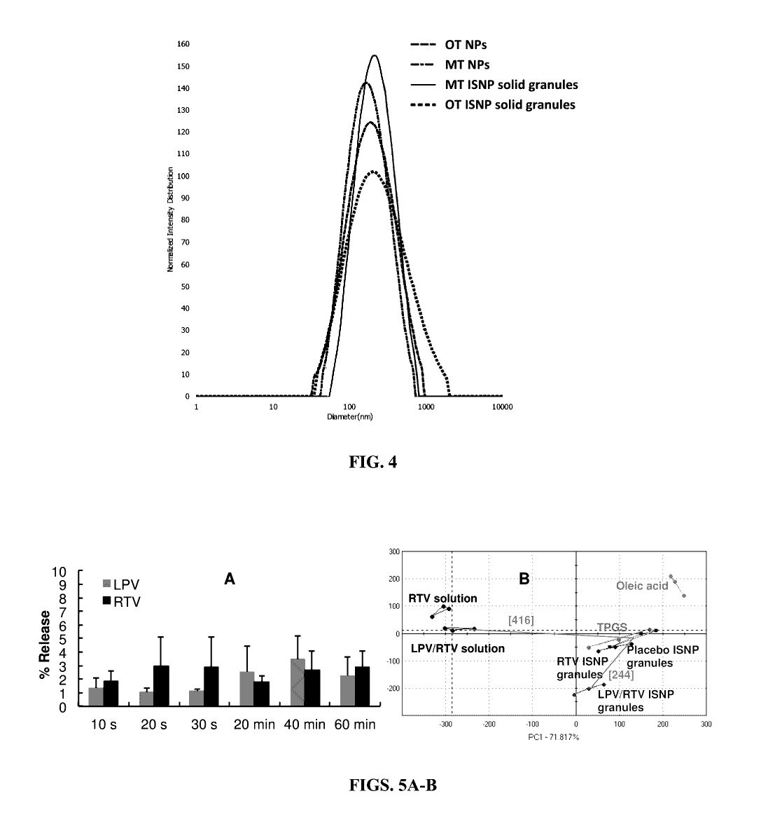

The inventor added drugs with the excipients at the beginning and followed mixing step as described above. An illustration of the preparation procedures is shown in FIGS. 1A and 1B. The detailed procedures and compositions are described in Examples 3, 5 and 6. The examples of particle size for the OT NPs, the MT NPs, the OT ISNPs, and the MT ISNPs are shown in FIG. 4 and Table 0.

TABLE 0
Particle size and size distribution of prototype nanoparticles

| Formulations | Size (nm) | P.I. | D (10%) | D (50%) | D (90%) |
|---|---|---|---|---|---|
| OT NPs | 161.0 | 0.293 | 72.2 | 180.2 | 445.0 |
| MT NPs | 148.1 | 0.230 | 75.3 | 162.3 | 359.4 |
| NPs from the | 190.3 | 0.203 | 99.9 | 204.3 | 420.9 |
| OT ISNP | | | | | |
| solid granules | | | | | |
| NPs from the | 214.3 | 0.297 | 74.3 | 213.8 | 696.4 |
| MT ISNP | | | | | |
| solid granules | | | | | |

P.I.: polydispersity index. P.I. <0.3 indicates monodispersed particles.
D (10%): 10% of nanoparticles have size smaller than this value.
D (50%): 50% of nanoparticles have size smaller than this value.
D (90%): 90% of nanoparticles have size smaller than this value.

### Example 2

As explained in the previous example, the inventor has developed a novel nanotechnology to prepare solid granules that produces ISNPs in contact with water with gentle agitation. They have utilized the nanotechnology to prepare RTV, LPV, LPV/RTV, and ATV/RTV/FDC/TDF solid ISNP granules. Pilot data from their laboratory demonstrated that these formulations were stable over time at room temperature and improved bioavailability compared to the controls. One goal is to implement this nanotechnology to formulate ATV/RTV and ATV/RTV/FDC/TDF oral disintegrating tablets (ODTs) for pediatric administration. With new ODTs, the inventor postulates that adequate systemic ATV exposure will be achieved, ATV bioavailability will be improved, and food effects of ATV will be reduced. Moreover, with the 4-drug combination ODTs, pill burden and drug cost will be reduced.

The inventor has developed RTV, LPV, LPV/RTV and ATV/RTV/FDC/TDF solid granules that spontaneously produced ISNPs in contact with water. Such NPs should improve bioavailability and reduce food effects. The preliminary data indicates that the novel ISNP nanotechnology improves bioavailability of LPV, RTV, ATV and TDF. Currently commercial ATV formulations are associated with food effects. In addition of PK, biodistribution and toxicity studies in rats, the inventor will use beagle dogs to evaluate food effects of ATV/RTV ODTs. To complete an ARV regimen, LPV/RTV should be taken with two NRTIs, e.g., 3TC and AZT, twice daily without regard to food intake. In addition, AZT while a first generation NRTI, with known resistance mutations, nevertheless represents one of the antiretroviral drug with good penetration of the blood brain barrier, and hence its continued use in cART. There are no drug-drug interaction among LPV, RTV, 3TC and AZT. Thus, the inventor will develop LPV, RTV, 3TC and AZT as the second PI-based FDC. Bitter taste and pill burden are the limitations of current LPV/RTV/3TC/AZT regimen. We will use LPV/RTV ISNPs to improve taste and decrease the drug doses and formulate the FDC to reduce pill burden. LPV/RTV/3TC/AZT FDCs will be heat stable, palatable and flexible for twice daily administration without the need to combine with food and with appropriate brain penetration. The inventor has demonstrated that the ISNPs can efficiently mask the taste of LPV and RTV. They will use clindamycin as a model drug to further develop the ISNP nanotechnology for taste masking. Handling compounded anti-cancer drugs for pediatric patients is problematic. The inventor will use methotrexate (MTX) to develop child-friendly and safe MTX ISNP granules and ODTs. The in vitro and in vivo studies proposed here will provide sufficient information to evaluate the potential of the proposed ARV formulations for clinical applications.

This unique nanotechnology represents a novel approach to overcome the challenges of using NPs for oral solid dosage forms. By completely removing water from NP preparation, the inventor dramatically increased drug loading and easily manufactured drug-loaded solid granules that produced self-assembly NPs in situ when introduced into water. Moreover, these were the first studies to encapsulate two PIs or four ARV drugs into the same NP formulation. The pilot in vitro and in vivo studies on RTV, LPV, LPV/RTV and ATV/RTV/FDC/TDF solid granules demonstrated the advantages of the novel ISNP nanotechnology. Second, masking drug taste by encapsulating drugs inside NPs is also an innovative method to improve palatability. Taste masking is essential for conventional ODTs because drugs are entirely released in the mouth. However, with ISNPs, drugs will be encapsulated in the NPs while the NPs are formed in the mouth, resulting in improved taste. Third, using in situ NPs is a truly new way to design ODTs by direct compression. Incorporation of NPs into ODTs by direct compression is challenging because conformation/properties of NPs would be changed by compression. However, direct compression will not affect the ISNPs. To the inventor's best knowledge, there are no reports about using in situ NPs to manufacture ODTs by direct compression. Finally, the improved bioavailability of PI-loaded solid granules resulted from the in situ-formed NPs; thus, dispersion in liquid will have minimal influence on the bioavailability. In other words, ATV/RTV ODTs will be reconstitutable, providing an opportunity to use the ODTs in infants. Importantly, the novel nanotechnology platform will be easy to be applied for other challenging drugs, potentially leading to multi-drug ARV combinations for non-adult populations and thereby providing an innovative platform for the development of new pediatric formulations. This nanotechnology takes the advantages of NPs as well as ODTs. With a single approach, the inventor can produce age-appropriate pediatric formulations (whether for infant, toddler or child).

Previously, the inventor developed and optimized nanocapsules composed of Brij 78, d-α-tocopheryl polyethylene glycol 1000 succinate (TPGS) and Miglyol 812 to encapsulate paclitaxel to overcome P-gp-mediated drug resistance in cancer (Dong et al., 2009) by using an emulsion method. The inventor has used the similar microemulsion procedure to develop new nanocapsules to load RTV, and then converted RTV nanocapsules to RTV nanoparticle-containing granules by wet granulation (FIG. 1A). With this conventional procedure, the inventor is still not possible to meet the dose requirement of RTV in one tablet, though the inventor obtained about 1% of RTV loading in the final granules.

TABLE 1
Properties of RTV, LPV and LPV/RTV ISNPs from reconstituted solid granules
(n = 3)

| Formulations | Particle size (nm) | P.I. | EE % | Zeta potential (mV) |
|---|---|---|---|---|
| RTV NPs from solid granules | 214 ± 14 | 0.292 ± 0.02 | 71.8 ± 4.8 | −52.9 ± 5.7 |
| LPV NPs from solid granules | 225 ± 18 | 0.305 ± 0.02 | 96.7 ± 5.7 | −18.7 ± 6.2 |
| LPV/RTV NPs from solid granules | 195 ± 25 | 0.276 ± 0.08 | 95.3 ± 4.2 (LPV) | −16.9 ± 2.5 |
| | | | 93.6 ± 5.4 (RTV) | |

Recently, the inventor developed a novel ISNP nanotechnology to prepare the pro-IS NP compositions by simply mixing oleic acid and melted TPGS. When the pro-ISNP composition was reconstituted into water under gentle shaking, the inventor obtained the nanocapsules as described above (FIG. 1B). They were able to load over 25% (w/w) of RTV or LPV into the pro-ISNP compositions. The inventor further converted the drug-loaded pro-ISNP to solid granules by mixing them with Avicel PH 102 or Aeropearl 300. The solid granules showed a very good flow. The stability data demonstrated that the RTV solid granules were physically and chemically stable at least for one month at RT. The drug loadings in the final solid powder were over 16%. Moreover, the inventor formulated both LPV and RTV into the same pro-ISNP compositions with a drug loading of 15.5% and 3.9%, respectively. After the inventor added water into the solid granules with gentle shaking, drug-loaded NPs formed and their properties were similar with the previous drug-loaded nanocapsules.

Flavors, sweeteners, coating and ion exchange resins have been used to mask unpleasant tastes of drugs; however, none of them could improve bioavailability for poorly water-soluble drugs. Lipid formulations, such as emulsion and microemulsion, are the most used technologies to improve bioavailability for poorly water-soluble drugs in commercial medications. However, high amounts of lipids and surfactants are required to form emulsions and microemulsions, which introduce unpleasant tastes and consequently limit lipid formulations for pediatric use. Particle size reduction and salt formation also are common technologies to solve solubility issues; however, with these technologies, drugs will dissolve in the mouth and generate unpleasant tastes. Solubility enhancers and absorption enhancers have been studied to improve oral bioavailability; however, the safety of excipients in children has been called into questions (Fasinu et al. 2011). Solid dispersion has been studied for taste masking or improved bioavailability; however, solid dispersion only could be used for drugs with melting points less than 200° C. Using lipid NPs is a new and promising way to combine the advantages of physical taste-masking methods, such as binding drug to an excipient and entrapping it in a particle. Lipids are hydrophobic making them excellent excipients for taste masking of drugs. The strengths of the ISNP nanotechnology is flexibility on formulation design and improved bioavailability. Both RTV and LPV are notorious for bad taste. The inventor has prepared LPV/RTV ISNP granules and measured drug release in PBS at pH 6.5 for 10-30 s to mimic drug dwelling time in the oral cavity while given by sprinkles, as well as for 20-60 min to mimic handling and administration time for sachets. Less than 3% of LPV or RTV released from the ISNP granules over 60 min, demonstrated the high taste-masking efficiency (FIG. 5A). To confirm the taste masking, we further used a sensor set array #2 (composed of ZZ, AB, GA, BB, CA, DA and JE 7 sensors) of an Astree E-tongue to evaluate the tastes. The principal component analysis (PCA) computes the data attained by all 7 sensors on the PCA chat, representing the largest (PC1, x-axis) and second largest (PC2, y-axis) relative contribution factors (FIG. 5B). It could be assumed that PC1 represents the intensity of bitterness. The taste comparison is based on the Euclidean distances between the center of the cluster of one formulation to the center of the cluster of the placebo ISNP granules on the PCA chart. As shown in FIG. 5B, the clusters of RTV and LPV/RTV ISNP granules are closer to the cluster of placebo ISNP granules compared to those from RTV and LPV/RTV solutions. Moreover, we calculated the Euclidean distances between formulation and the placebo using “Masking Efficiency” analysis. As shown in FIG. 5B, the distance of LPV/RTV ISNP granules (244) is shorter than that of LPV (416), indicating similar tastes of LPV/RTV ISNP granules to the placebo. Oleic acid is the major component in olive oil, and TPGS is used as a supplement of vitamin E in nutrition without complaint on taste. Thus, the placebo ISNPs should not have taste issues in humans. Overall, the data demonstrate that the ISNP granules sufficiently masked the tastes of LPV and RTV.

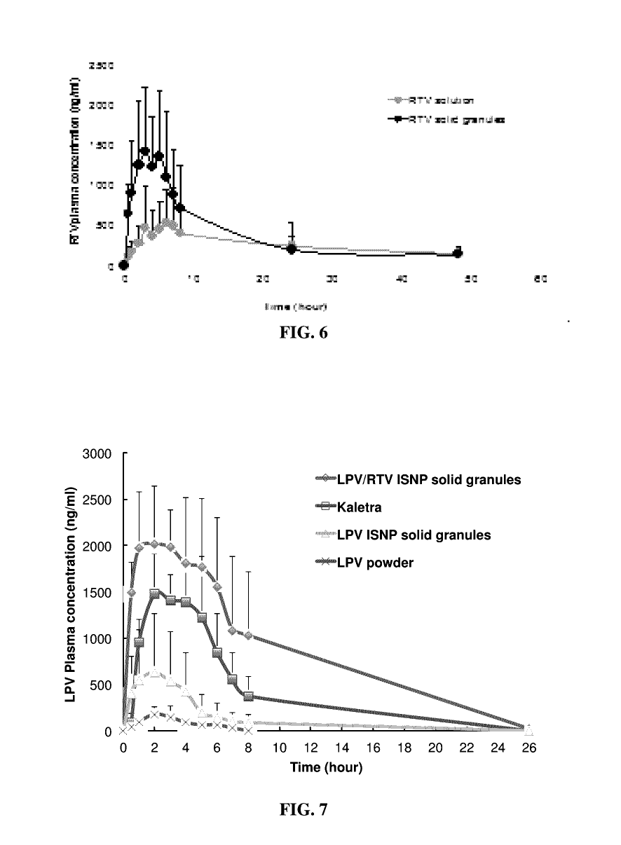

The inventor has also compared the PK of RTV ISNP granules, LPV ISNP granules, LPV/RTV ISNP granules and ATV/RTV/FTC/TDF ISNP granules with the marketed formulations. As shown in FIGS. 6 and 7, ISNP-based solid granules increased the bioavailability of RTV and LPV/RTV. The relative bioavailability of RTV solid granules and LPV/RTV solid granules was 230±104% and 203.9±94%, respectively, compared to the commercial formulations. Moreover, LPV/RTV ISNP solid granules maintained LPV plasma concentration >1000 ng/ml over 8 hours. In contrast, LPV ISNP solid granules did not achieve adequate LPV plasma concentration though increased the bioavailability compared to LPV powder. In the literature, LPV solid lipid NPs, LPV nanocrystals and LPV polymer NPs improved the AUCs without co-administration of RTV; however, they did not maintain adequate PI plasma concentrations over 8 hours. It is also noteworthy that none of these PI NPs were developed as final oral solid dosage forms. In Kaletra, LPV plasma concentration maintained over 8 hours (FIG. 7). Noticeably, the purpose of adding RTV was not only to enhance bioavailability (AUC) but also to maintain consistent PI plasma concentration over time 14, which is more important for HIV treatment because the HIV inhibitory quotient (IQ) is calculated based on C_{trough}—the lowest concentration required for virological response (Becker, 2003 and Boffito et al., 2008). Moreover, the ISNP granules increased the bioavailability for ATV and TDF (FIG. 8) in the ATV/RTV/FTC/TDF FDC granules. Therefore, the novel ISNP nanotechnology can improve oral bioavailability for various drugs.

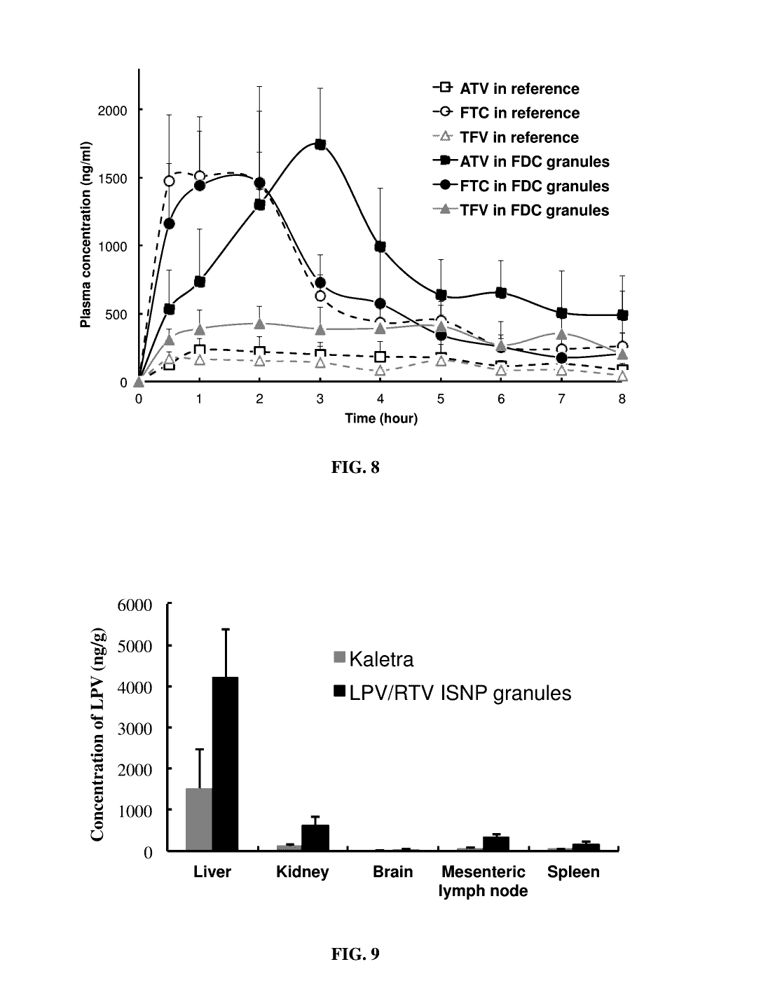

Brain and lymphoid tissues constitute major sanctuary sites for HIV because they are poorly perfused organs and stringent exclusion barriers to many conventional therapies. Lipid NPs can be taken by Peyer's patches or as chylomicron-like particles in the intestine to enter the lymphatic circulation. The inventor studied the biodistribution of Kaletra® and LPV/RTV ISNP granules (FIG. 9). As compared to Kaletra®, the granules significantly increased LPV concentrations in all tested tissues.

Preparation and Characterization of ATV/RTV Solid Granules by the ISNP Nanotechnology:

One of the limitations associated with ATV-based HIV treatment is the food effect, which decreases the convenience of the administration and consequently reduces patient adherence. The ISNP nanotechnology fundamentally solves the solubility issue, and consequently could avoid food effects. The inventor will develop ATV/RTV ISNP granules to avoid the food effect of ATV. They will first measure the solubility of ATV in FDA-approved lipids and surfactants, such as glyceryls, fatty alcohols, Myrijs, pluronics, Tweens and phospholipids. Then, they will prepare ATV/RTV ISNP solid granules containing a 3:1, 4:1 or 5:1 ratio of ATV and RTV for the PK studies in rats. The ATV/RTV ISNP granules will be prepared as described in Example 1. Briefly, ATV and RTV will be mixed with melted excipients at 60-70° C. for 10 min to prepare pro-ISNPs. And then absorption carriers will be added into the pro-ISNPs. After cooling, the inventor will obtain ATV/RTV solid granules. Different solid carriers, such as Avicels, Aeropearls, lactose and dextran, will be tested to produce optimal flowability and drug loading. Once the solid granules are dispersed into water with gentle agitation, ATV/RTV NPs will be spontaneously produced. The inventor will fully characterize the NPs and all characterization methods for drug-loaded NPs were established in their previous studies (Pham et al., 2016, Guo et al., 2016). To characterize the ATV/RTV solid granules, the inventor will measure the manufacturing yield to evaluate the loss of the material, partially indicating the flowability of the final solid granules. The inventor will measure angle of repose, the bulk density and the tapped density, and Hausner ratio to evaluate flowability and compressibility of the solid granules. To understand food effects, they will perform two-stage dissolution studies under fasted and fed conditions. For the fasted state, they will start the study in fasted-state simulated gastric fluid for 2 hours and then switch to fasted-state simulated intestinal fluid for another 6 hours. For the fed state, the study will be in fed-state simulated gastric fluid and then in fed-state simulated intestinal fluid. As quality control, they will measure two-stage dissolution in the SGF and in pH 6.8 medium as mentioned above. The solid granules will be stored at RT over time, and the stability of the solid granules will be tested according to appearance, particle size, zeta potential, drug content (including degradation monitoring), EE % and dissolution. They will mix the ATV/RTV solid granules with other excipients, e.g. flavorants, sweetener, citric acid and sodium carbonate (effervescent disintegration pair to assist disintegration and masking taste), crospovidone (superdisintegrant to provide fast disintegration), and Avicel (to adjust compressibility), and talc (lubricant to improve flowability for tablet compression). The homogenous blend will then be compressed to ODTs by direct compression using a MiniPress (SMI, NJ). The compression parameters will be adjusted to fill the die with correct weights and to compress the tablets with a desired hardness. The final tablet weight is <1 gram, and friability is <2% for 100 rotations. The inventor will prepare three different ODTs using the selected solid granules for analytical tests and taste evaluation. In addition to PK, biodistribution and toxicity studies in rats, we will use beagle dogs to evaluate food effects of ATV/RTV ODTs.

Preparation and Characterization of ATV/RTV/FTC/TDF FDC ODTs by the ISNP Nanotechnology.

The inventor developed ATV/RTV/FTC/TDF FDC ISNP granules and studies their PK profile. Furthermore, they will compress the FDC granules to FDC ODTs for once daily dosing to cover children in different ages. They will prepare the FDC ISNP granules as described in Example 1. Then, they will add other solid excipients to compress the FDC granules to the FDC ODTs and characterize them as described above. Finally, they will compare the PK profiles of the FDC granules and the FDC ODTs.

Preparation and Characterization of LPV/RTV/3TC/AZT FDC Granules and ODTs by the ISNP Nanotechnology:

LPV/RTV oral solution is the only combination of PIs. Because of poor taste, the oral solutions are mixed with chocolate milk prior to administration and juice, jelly and strong flavors are given after each dose. Additionally, the LPV/RTV oral solution requires refrigeration for storage and transportation. Patient adherence is a major problem for the regimens containing LPV/RTV oral solution, especially in resource-limited settings. This aim will utilize LPV/RTV pro-ISNP to solve the taste and cold-chain issues and combine them with 3TC and AZT to reduce pill burden. They will prepare the FDC ISNP granules as described in Example 1. Then, they will add other solid excipients to compress the FDC granules to the FDC ODTs and characterize them as described above. Finally, they will compare the PK profiles of the FDC granules and the FDC ODTs.

Optimization of ISNP Compositions for Improved Taste Masking by Using the ISNP Nanotechnology: The Inventor Will Use Clindamycin as a Model Drug for this Study.

Clindamycin free base, an amorphous form, has low solubility and high permeability (BCS II). To improve solubility, pro-drugs, e.g., its hydrochloride (HCl) salt, were developed and are used in commercial medications. Clindamycin HCl is a BCS I drug with a bioavailability of 90%. In the commercial pediatric solution, clindamycin HCl is completely dissolved in the solution, leading to perceptible exposure of the drug to the taste buds—extremely bitterness. Numerous liquid compositions of clindamycin HCl have been studied; however, the stability and taste still are problematic. As a result, there are no palatable and easy-swallowing clindamycin formulations commercially available. On the other hand, during drug product development, it is very often difficult to develop suitable salt forms. Intensive efforts and time have to be put on salt screening and polymorphism studies. We will use the ISNP nanotechnology to formulate clindamycin free base for taste masking along with bioavailability improvement. The ISNP granules will solve the solubility issue of clindamycin free base to provide sufficient bioavailability. At the same time, the taste of clindamycin free base will be efficiently masked by encapsulating it into the ISNPs. First, they will test the solubility of clindamycin free base in FDA-approved excipients. These lipids and surfactants are widely used in foods without causing taste problems. The excipients that show high solubility will be selected. Then, they will find a composition for placebo pro-NPs with two components (one lipid and one surfactant). They will mix clindamycin free based with pro-NP to prepare the ISNP granules as described in Example 1.

They Will Add Three New Approaches to Further Enhance Taste Masking.

Firstly, the studies showed that lecithin blocks bitter taste by directly binding lecithin with gustatory cell membrane (Zidan 2016). They will incorporate lecithin into the placebo ISNPs to develop novel lecithin-enhanced ISNPs that can block bitterness. Secondly, polymers can directly bind small molecules via a number of non-covalent interactions (Madgulkar, Bhalekar et al. 2009). In particular, Eudragit E PO is insoluble in the mouth (pH 5-7), but readily soluble in the stomach (pH 1-4). Thus, they will add Eudragit E PO into the placebo ISNPs to prepare novel polymer-enhanced ISNPs, which could enhance entrapment, bind bitter drugs and further control drug release. Finally, the surface of solid carriers contains different functional groups that control the formation of hydrogen bonds with entrapped materials. They observed the different release rates of drug-loaded ISNPs by using Avicel PH 105 and Aeropeal 300. Thus, they will test different solid carriers to control the release of materials from the solid ISNP granules, consequently controlling the drug release in the mouth. They will combine these new approaches to maximize the efficiency of taste masking.

Preparation and Characterization of Children-Friendly and Safe MTX ODTs by Using the ISNP Nanotechnology:

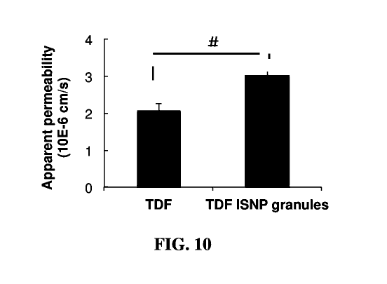

Because the ISNPs are formed after the granules are in contact with water, the compress force will have little or no influence on the ISNP formation. The taste of the drug will be masked by the ISNP granules; thus, ODT technology and taste masking technology are combined in our novel ODTs. MTX does not have long-term stability in aqueous solutions. To date, the only pediatric oral solution in literature was recently developed as a compounding solution by the pharmacists in the hospital, which was stable for 120 days (Vrignaud, Briot et al. 2015). MTX possesses low permeability (a BCS III drug). The oral bioavailability of MTX is on average 30%. Since MTX is cytotoxic, safety handling must be considered when developing flexible and reconstituted solid dosage forms. The ISNPs will protect MTX from enzyme degradation and precipitation in the GI tract. According to the inventor's preliminary data, the ISNP granules increased TDF (a BCS III drug) bioavailability (FIG. 8). Their further studies showed that the ISNPs improved cell permeability of TDF in a Caco-2 model (FIG. 10). Because lipid NPs enter cells by endocytosis (Dong 2009), the inventor can use the ISNPs to change MTX uptake pathway and consequently avoid the saturation of the transporter. Moreover, the ISNPs can be taken by the lymphatic systems (FIG. 9). Thus, the ISNP nanotechnology has great potential to increase bioavailability for low-permeable drugs.

The inventor will screen FDA-approved lipids and surfactants to select optimal excipients to prepare pro-NPs for MTX. MTX ISNP granules will be prepared as described in Example 1. Commercial MTX tablets are in a range of 5-15 mg per tablet; thus, they will set up the target at 15 mg MTX in 300 mg granules. MTX is a cytotoxic drug. To safely use the granules by humans, they will coat a polymer outside the granules by a Micro Flo-coater (Fraud-vect, IA). FDA-approved polymers will be optimized to efficiently release ISNPs. Particle size and dissolution will be studied for coated and uncoated granules to evaluate and adjust the influence of coating. Since the purpose of coating is only for the isolation of the drug during handling, 2-3% coating will be enough, which will be quick and simple. Once drug-loaded solid granules are dispersed into water with gentle agitation, MTX-loaded ISNPs will be spontaneously produced. Stability and characterization will be followed as described in the previous studies (Guo et al., 2016; Pham et al., 2016). The taste of MTX ISNP granules will be compared with a commercial capsule formulation. The coated MTX ISNP granules will be compressed to the ODTs as described above. The rat studies will be performed to compare the PK profiles of MTX ODTs and commercial MTX tablets.

### Example 3

Ritonavir (RTV) is a protease inhibitor for HIV treatments. However, it is not used as a therapeutic agent, but a “boost” agent to inhibit enzyme CYP3A4 and P-glycoprotein to enhance the bioavailability of other HIV drugs. Currently commercial RTV formulations include tablets, capsules and solution. Only the solution is suitable for pediatric patients. RTV is not water-soluble. Based on solubility measurements, the saturated solubility of RTV in the simulated gastric fluid at 37° C. is about 0.2 mg/ml. Lopinavir is another protease inhibitor with poor water solubility. It has to be used with RTV to achieve adequate bioavailability. The RTV or LPV/RTV solution is composed of 42.4% ethanol and 15.3% propylene glycol. RTV is very bitter. Once it dissolved in the high amounts of ethanol and propylene glycol, the RTV solution has very unpleasant taste. Moreover, RTV is not stable in the solution for a long time; thus the storage condition for the RTV solution is 6 months at 4° C. With these limitations on taste and cool-chain storage, the use of the RTV solution is problematic. Therefore, new RTV pediatric formulations are urgent needs to improve palatability and stability. At the beginning, the inventor developed RTV OT NPs by using an emulsion method. After preparation, the RTV OT NPs was in water as suspensions. The inventor has tried to convert the NP suspension to solid dosage forms by wet granulation in order to improve the stability. However, they observed low drug loading (˜1.2%).

Recently, they developed the ISNP nanotechnology discussed in Example 1 and completely removed water from the NP preparation. They are able to easily convert the RTV OT ISNPs to solid granules. In a comparison, the properties of the RTV OT NPs and the RTV OT ISNP solid granules are comparable. By using the ISNP nanotechnology, they also developed LPV and LPV/RTV OT ISNP solid granules.

Solubility Screening for RTV and LPV.

These studies were performed to test the solubility of RTV in oils and surfactants. Briefly, oils and surfactants were accurately weighed into glass vials. Certain amounts of CUR were added into the glass vials. The vials were stirred on the hot plate maintained at 65° C. for 3 min. The solubilization was monitored by eyes and the drug solubility (w/w) was estimated. The results are shown in Table 2 for RTV and in Table 3 for LPV. According to this study, oleic acid and TPGS were selected for RTV and LPV nanoformulations.

TABLE 2
Soluble of ritonavir in oils and surfactants

| Excipents | Esimated ritonavir solubility (mg/mg) |
|---|---|
| Oleic acid | 0.432 |
| Miglyo812 | 0.0020 |
| Kollisolv MCT70 | 0.0150 |
| Glyceryl behenate | 0.1720 |
| Kolliwax GMS I | 0.1360 |
| Killiphor P 188/Poloxamer F68 | 0.0630 |
| TPGS | 0.0540 |
| Brij 78 | 0.0530 |

TABLE 3
Soluble of lopinavir in oils and surfactants

| Excipents | Esimated Lopinavir solubility (mg/mg) |
|---|---|
| Oleic acid | 1.19 |
| Miglyo812 | 0.0703 |
| Kollisolv MCT70 | 0.0779 |
| Glyceryl behenate | 0.0744 |
| Kolliwax GMS I | 0.5846 |
| Killiphor P 188/Poloxamer F68 | 0.3431 |
| TPGS | 0.6012 |
| Brij 78 | 0.4725 |

Preparation of RTV OT NPs, RTV OT ISNPs, LPV ISNPs and LPV/RTV ISNPs.

The NPs were prepared by using an emulsion method and the ISNPs were prepared by using the pro-ISNP composition nanotechnology as described in Example 1. To load drugs, the drugs were weighed and added into the melted oleic acid and TPGS and then the inventor followed the rest procedure for the prototype NPs or ISNPs.

Preparation of RTV OT ISNP Solid Granules, LPV OT ISNP Solid Granules, and LPV/RTV OT ISNP Solid Granules.

Briefly, drugs were mixed with melted TPGS and oleic acid at 60° C. for 10 min. an adsorption agent, such as Avicel PH 102 and Aeropearl 300, was added into melted mixture and cooled down to room temperature with stirring. Then, the inventor got the solid granules. The example compositions of drug nanoformulations are shown in Table 4. The inventor can make the nanoformualtions by proportionally change the amount of each component.

TABLE 4
Composition of RTV OT NPs, RTV ISNP solid granules, LPV ISNP solid
granules and LPV/RTV ISNP solid granules

| | | | Oleic | | | |
|---|---|---|---|---|---|---|
| | LPV | RTV | acid | TPGS | Water | Aeropearl |
| Formulations | (mg) | (mg) | (mg) | (mg) | (ml) | 300 (mg) |
| RTV OT NPs | | 0.6 | 2 | 4 | 1 | — |
| RTV OT ISNP solid | — | 50 | 50 | 100 | — | 110 |
| granules | | | | | | |
| LPV OT ISNP solid | 50 | — | 50 | 100 | — | 110 |
| granules | | | | | | |
| LPV/RTV OT ISNP | 50 | 12.5 | 50 | 100 | — | 110 |
| solid granules | | | | | | |

Characterization of RTV OT NPs, RTV OT ISNP Solid Granules, LPV OT ISNP Solid Granules and LPV/RTV OT ISNP Solid Granules.

The measurement methods for RTV OT NPs are similar with those for DTX MT NPs and CUR MT NPs in Examples 6 and 7. For drug ISNP solid granules, the measurement methods were described in Example 2. Table 5 contains updated information for the Table 2 in Example 2.

TABLE 5
Summary of physiochemical properties of RTV OT NPs, RTV ISNP solid
granules, LPV ISNP solid granules and LPV/RTV ISNP solid granules

| | Theoretical | Measured drug | Mean diameter | | % Drug Entrapment |
|---|---|---|---|---|---|
| Formulations | drug loading % | loading % | (nm) | P.I. | Efficiency |
| RTV OT NPs | 4.8 | 4.6 ± 0.05 | 239.5 ± 23.8 | 0.281 ± 0.017 | 93.7 ± 2.0 |
| RTV ISNP solid granules | 16.1 | 17.8 ± 2.3 | 160.4 ± 7.1 | 0.282 ± 0.009 | 70.2 ± 0.7 |
| LPV ISNP solid granules | 16.1 | 14.6 ± 0.8 | 154.3 ± 8.2 | 0.200 ± 0.016 | 95.2 ± 3.8 |
| LPV/RTV ISNP solid granules | 16.1 (LPV) | 14.4 ± 1.8 (LPV) | 157.6 ± 24.1 | 0.203 ± 0.033 | 101.4 ± 4.8 (LPV) |
| | 3.5 (RTV) | 3.2 ± 0.6 (RTV) | | | 103.1 ± 5.9 (RTV) |

To evaluate stability of particle size at physiological condition, 20 mg of solid granules (about 13 mg of ISNPs) were incubated in 11 ml of the simulated gastric fluid (pH 2) for 2 hours. Then, 0.5 M NaOH and 2M KH₂PO₄ were added into the SGF to adjust pH to 7. The solid granules were continually incubated for another 6 hours. At predetermined time points, the inventor withdrew 1.5 ml of medium, centrifuged and took 1.1 ml of supernatant to measure particle size. The results are shown in FIG. 2. Apparently, in situ NPs formed after the inventor put solid granules into the SGF. Particle size of the in situ NPs had no significant difference compared to the initial at time 0 (p>0.05). The data in FIG. 2 also demonstrate that ISNPs are different from SMEDDS and SNEDDS because both microemulsions and nanoemulsions cannot be stable at this kind of low concentration over 8 hours.

The inventor measured release profiles of drug loading nanoformulations. Also, see FIG. 3A for the release profiles of RTV OT NPs and RTV OT ISNP solid granules, and see FIG. 3B for the release profiles of LPV/RTV ISNP solid granules. Currently, the inventor has six-month stability at room temperature for RTV OT ISNP solid granules (Table 6), LPV OT ISNP solid granules (Table 7A) and LPV/RTV OT ISNP solid granules (Table 7B).

TABLE 6
Long-term stability of RTV OT ISNP solid granules at room temperature

| Parameters | 2 weeks | 5 weeks* | 14 weeks* | 24 weeks* |
|---|---|---|---|---|
| Measured drug loading % | 15.3 ± 0.6 | 15.7 ± 1.1 | 15.4 ± 0.7 | 15.1 ± 0.2 |
| Degradation % | 0 | 0 | 0 | 0 |
| EE % | 70.2 ± 7.1 | 71.5 ± 6.6 | 59.3 ± 6.2 | 63.8 ± 2.2 |
| Mean diameter (nm) | 300.2 ± 27.3 | 290.3 ± 18.9 | 251.9 ± 9.9 | 267.9 ± 22.8 |
| PI | 0.311 ± 0.01 | 0.305 ± 0.01 | 0.276 ± 0.01 | 0.307 ± 0.01 |
| Zeta potential (mV) | −42.9 ± 12.3 | −38.6 ± 8.1 | −57.4 ± 2.5 | −39.9 ± 2.0 |

TABLES 7A-B
Long-term stability of LPV and LPV/RTV OT ISNP solid granules

| | | | | |
|---|---|---|---|---|
| Parameters | One month | Two months | Three months | Six months |

A. LPVISNP granules

| | | | | |
|---|---|---|---|---|
| Measured DL % | 13.8 ± 0.6 | 14.2 ± 0.4 | 13.1 ± 0.4 | 15.5 ± 1.31 |
| Degradation % | 0 | 0 | 0 | 0 |
| EE % | 98.9 ± 2.8 | 101.2 ± 4.0 | 97.6 ± 3.5 | 98.7 ± 2.9 |
| Particle size (nm) | 166.2 ± 6.6 | 162.8 ± 9.6 | 143.6 ± 7.6 | 171.0 ± 14.7 |
| P.I. | 0.210 ± 0.020 | 0.100 ± 0.023 | 0.250 ± 0.021 | 0.235 ± 0.027 |

B. LPV/RTV ISNP granules

| | | | | |
|---|---|---|---|---|
| Measured DL % | 14.1 ± 1.4 (LPV) | 15.2 ± 1.9 (LPV) | 14.1 ± 1.9 (LPV) | 15.4 ± 2.9 (LPV) |
| | 3.6 ± 0.3 (RTV) | 4.4 ± 0.5 (RTV) | 3.1 ± 0.4 (RTV) | 3.4 ± 0.7 (RTV) |
| Degradation % | 0 | 0 | 0 | 0 |
| EE % | 95.6 ± 5.6 (LPV) | 87.0 ± 4.1 (LPV) | 98.9 ± 1.7 (LPV) | 96.3 ± 3.6 (LPV) |
| | 96.2 ± 3.2 (RTV) | 91.9 ± 5.0 (RTV) | 93.9 ± 2.2 (RTV) | 100.1 ± 0.8 (RTV) |
| Particle size (nm) | 158.9 ± 8.7 | 150.8 ± 6.3 | 144.4 ± 17.6 | 161.3 ± 30.9 |
| P.I. | 0.244 ± 0.039 | 0.200 ± 0.023 | 0.272 ± 0.012 | 0.263 ± 0.020 |

The pharmacokinetic studies of RTV OT ISNP solid granules are shown in FIG. 6. The pharmacokinetic studies of LPV/RTV OT ISNP solid granules and LPV OT ISNP solid granules are shown in FIG. 7.

### Example 4

Many diseases, such as HIV and tuberculosis, require administration of multiple drugs. FDCs make it possible to combine two or more drugs with different modes of pharmacological actions in a single dosing unit. The US guidelines recommend that one complete cART for HIV-infected children comprise two-nucleoside reverse transcriptase inhibitors (NRTI) such as FTC and TDF, plus a PI such as RTV-boosted LPV or ATV. However, the poor palatability of the RTV oral solution, and pill burdens become significant challenges on medication adherence for this regimen. Single-unit regimen, in which patients take one unit of a FDC to finish one complete cART, has been demonstrated to improve patient adherence and virologic response in adult patients (Food and Drug Administration, 2006). Compared with separate drugs, FDCs not only reduce pill burden but also decrease manufacturing and distributing costs. Patient compliance is pivotal to avoid development of drug resistance. However, most combinations are in adult formulations (tables or capsules). The main challenge on developing FDCs usually involves product formulation and manufacturing issues. Drugs in the FDCs have to be physically and chemically compatible along with their excipients. When the drugs contain different physicochemical properties (different BCS classes), the formulation technology is required to improve both solubility and permeability. In addition, the formulation technology also needs to mask the taste of drugs for pediatric FDCs. Moreover, the costs of pediatric formulations must be considered during development. The difficulty of formulation development could result in higher costs of pediatric formulations compared to existing adult formulations. This economic barrier of pediatric formulations could limit the clinical applications. Unfortunately, there are no such “super” formulation technologies available for pediatric FDCs. This leads to more challenges on the development of pediatric FDCs. As a result, very few pediatric FDCs are commercially available.

The inventor has recently developed a FDC ISNP granule containing 4 HIV drugs: ATV (BCS II), RTV (BCS IV), FTC (BCS I) and TDF (BCS III). The 4-drug FDC ISNP granules will achieve a single-unit regimen for one dosing per day. This study established the ISNP nanotechnology for FDC granules containing drugs in different BCS categories. The inventor will further develop the FDC granules to FDC ODTs to improve medication adherence in adolescent. Thus, they will develop child-friendly FDC granules and ODTs from one manufacturing process to provide suitable dosage forms for children of all ages. The manufacturing of the granules and the ODTs is simple and scalable. Moreover, the 4-drug FDCs will greatly reduce pill burdens. Therefore, costs of formulations, one of the major burdens of current HIV treatment, will be significantly reduced by the novel ISNP nanotechnology.

Methods.

The inventor prepared the ISNP granules as described in Example 2 with different drugs. The proportion of each drug in the FDC ISNP granules was designed to give 10 mg ATV/4 mg RTV/6 mg FTC/8 mg TDF per kg for once-daily dosing. Briefly, ATV, RTV, FTC, TDF, oleic acid, TPGS were added into a glass vial and mixed at 60° C. for 10 min. Then, an absorption agent, such as Avicel PH 102 and Aeropearl 300, was added into the mixture and cooled down to room temperature. Then, the inventor got the solid granules. The example compositions of FDC nanoformulations are shown in Table 8.

TABLE 8
Composition of ATV/RTV/FTC/TDF ISNP solid granules

| | | | | | Oleic | | Aeropearl |
|---|---|---|---|---|---|---|---|
| | ATV | RTV | FTC | TDF | acid | TPGS | 300 |
| Formulations | (mg) | (mg) | (mg) | (mg) | (mg) | (mg) | (mg) |
| ATV/RTV/ | 40 | 16 | 32 | 24 | 12.5 | 25 | 8 |
| FTC/TDF | | | | | | | |
| ISNP | | | | | | | |
| solid granules | | | | | | | |

The FDC ISNP granules were characterized for particle size, DL %, EE % and stability at room temperature as described in Example 2. The inventor administered rats a dose equivalent to 10 mg/kg ATV in the reference by oral gavage, which is corresponding to the recommended dose of 200 mg ATV in a child weighing 20 kg. To test the permeability of the ISNPs, the inventor conducted the permeability of TDF ISNPs in a Caco-2 model.

Results.

The FDC did not change the physiochemical properties of the RTV ISNPs in terms of particle size (148±17 nm), P.I. (0.26±0.04) and EE % (85.7±11.7%). Interestingly, over 80% ATV, 40% FTC and 40% TDF were also entrapped into the ISNPs. The FDC ISNP granules were stable at room temperature at least for one month (Table 9). The PK study showed that ATV/RTV/FTC/TDF granules increased the bioavailability of ATV (2.3 folds), RTV (2 folds), and TDF (3 folds) as well as kept the bioavailability of FTC compared to the reference (a mixture of commercial drugs) (FIG. 8). ATV is poorly water-soluble and has about 20% bioavailability without food. The FDC granules improved the bioavailability of ATV, properly by improving the solubility of ATV and the bioavailability of RTV. TDF has low permeability and is degraded by the enzymes in the GI tract, leading to 25% bioavailability. The ISNPs increased the TDF bioavailability by improving cell permeability (FIG. 10), and properly by protecting TDF from the degradation and improving the TDF uptake through the lymphatic systems. FTC is a BSC I compound with over 98% bioavailability; the FDC granules kept the FTC bioavailability. These preliminary data demonstrate the application of the ISNP nanotechnology for 4-drug FDCs, and also the ability of the ISNPs to enhance drug permeability. The FDC ISNP granules could reduce the required doses of ATV, RTV and TDF in terms of improved bioavailability, consequently reducing toxicities of these drugs.

TABLE 9
Long-term stability of ATV/RTV/FTC/TDF OT ISNP solid granules

| | day 0 | | | | One month | | | |
|---|---|---|---|---|---|---|---|---|
| Parameters | ATV | RTV | FTC | TDF | ATV | RTV | FTC | TDF |
| Theoretical DL % | 25.4 | 10.2 | 20.3 | 15.2 | 25.4 | 10.2 | 20.3 | 15.2 |
| Measured DL (%) | 22.9 ± 0.9 | 8.0 ± 0.7 | 18.9 ± 0.8 | 14.0 ± 0.4 | 22.8 ± 1.1 | 8.4 ± 1.0 | 20.9 ± 0.7 | 14.0 ± 0.2 |
| % drug AUC in HPLC | 99.3 | 99 | 99.2 | 99.1 | 99.2 | 99.3 | 99.1 | 99.5 |
| EE (%) | 91.0 ± 9.8 | 85.7 ± 11.7 | 33.9 ± 6.4 | 40.4 ± 7.6 | 83.0 ± 7.9 | 97.7 ± 3.4 | 40.3 ± 2.4 | 48.1 ± 0.4 |

| | | |
|---|---|---|
| Particle size (nm) | 148 ± 17 | 176 ± 25 |
| P.I. | 0.26 ± 0.04 | 0.29 ± 0.02 |

### Example 5

Human immunodeficiency virus (HIV) is one of the major global public health issues with approximately 36.7 million people living with HIV and 1.1 million people died from AIDs-related illness worldwide in 2015, according to the World Health Organization (WHO) (2016). There is no cure for HIV infection. However, HIV infection can be treated and prevented from developing into AIDS by using oral HIV medications called antiretroviral therapy (ART). Even though medications can improve the survival of persons infected with HIV, there are non-adherence issues such as forgetting to take HIV medications, side effects, and pill burden (Chesney, 2000; Adefolalu and Nkosi, 2013; Johnson and Neilands, 2007). Also, at least 95% adherence is necessary for fully effective HIV viral suppression (Shuter, 2008).

LPV is one of the potent protease inhibitors (PIs) used for the treatment of HIV infection. LPV has very poor bioavailability when it is administered orally due to high first pass metabolism, primarily mediated by the cytochrome 450 3A4 (CYP3A4). To solve this bioavailability problem, RTV is co-administered with LPV by inhibiting the CYP3A4-mediated metabolism of LPV. Both LPV and RTV are poorly water-soluble drugs. Kaletra® is once a day combination pill of LPV/RTV at a 4:1 ratio (LPV/RTV, w/w). Still, the use of taking oral LPV/RTV pill does not address the issue of non-adherence to ART. Moreover, the potential side effects for LPV/RTV are gastrointestinal disturbance, such as nausea, vomiting, and diarrhea (Kaletra, 2015).

Long-acting injectable nanoformulations for ART offers alternative therapeutic options for the treatment of HIV. Injectable nanoparticle has the potential to improve the pharmacokinetic properties of drug molecules and overcome GI side effects (Margolis and Marta, 2015), therefore it will likely improve drug adherence. Furthermore, long-acting injections are not influenced by first-pass pass metabolism, decreasing the potential for drug-drug interactions. Recently, the inventor has developed the ISNP granules that were capable of encapsulating both LPV and RTV with over 95% entrapment efficiency (EE) and physically stable over 6 months at room temperature (Pham et al., 2016). The LPV/RTV ISNP granules increased the bioavailability of LPV over 2.5 fold compared with a commercial LPV/RTV tablet. Interesting, the inventor also obtained excellent nanoparticles by reconstituting pro-NPs, an intermediate in the preparation of the ISNP granules.

The purpose of this study was to develop a novel long-acting injectable nanoformulation to encapsulate LPV and RTV by using the ISNP nanotechnology. FDA approved D-α-tocopherol polyethylene glycol 1000 succinate (TPGS) and oleic acid excipients were chosen as surfactant and lipid, respectively. Long-acting ISNPs were characterized in terms of particle size, EE, drug loading (DL), in vitro release study and pharmacokinetic study.

A. Materials and Methods

Preparation of ISNPs.

NPs were prepared using an ISNP method as described in Example 2. Briefly, 50 mg of oleic acid, 100 mg of TPGS, 50 mg of LPV, and 12.5 mg of RTV (LPV: RTV=4:1, w/w) were weighed and heated in a glass vial at 50° C. for 10 min and cooled to room temperature to form pro-NP. Then, 1 ml of 0.9% NaCl was added into pro-NP and vortexed to obtain LPV/RTV ISNPs.

Characterization of LPV/RTV ISNPs.

To determine particle size and size distribution, 100 μl of LPV/RTV ISNPs were diluted with 1 ml of Milliq water and then measured using a Delsa Nano HC Particle Analyzer (Beckman Coulter) at 165° light scattering at 25° C.

Determination of Entrapment Efficiency and Drug Loading.

To measure drug entrapment efficiency in the ISNPs, free LPV and RTV were separated from the ISNPs using a membrane filtration device (Microcon Y-100) by centrifugation at 14,000 rpm for 5 min at 4° C. Then, free LPV and RTV were measured by a HPLC method using a Waters Alliance HPLC System and an Inertsil ODS-3 column (4.6×150 mm) (GL Sciences Inc.). The HPLC mobile phase consisted of acetonitrile-0.1% TFA (70:30, v/v) at flow rate of 0.5 ml/min with detection at 210 nm. Entrapment efficiency was calculated as follows:

% drug entrapment efficiency=(drug entrapped in ISNPs/total drug in ISNPs)×100% (w/w)

To determine drug loading, 1 part of LPV/RTV ISNPs was dissolved in 33.3 parts of methanol by aggressively vortexing for 5 min for HPLC measurement. Drug loading was calculated as follows:

% drug loading=[drug in ISNPs/total weight of (drug+excipients)]×100% (w/w)

In Vitro Release Studies.

LPV/RTV release studies (n=4) were completed at 37° C. using PBS buffer as the release medium. 200 μl of LPV/RTV OT NPs were added into 20 ml PBS and shaken at 135 rpm at 37° C. 500 μl of sample were withdrawn at predetermined time intervals, and added into a Microcon Y-100 to separate the released drugs from the NPs as described above. Then, 400 μl of MeOH were added into the membrane to destroy LPV/RTV ISNPs and collect the drugs. The concentration of LPV and RTV were determined by using HPLC as described above.

Pharmacokinetic Studies.

Animal studies were performed in compliance with guidelines set by the University of North Texas Health Science Center Institutional Animal Care and Use Committees. Rats were housed in groups of 2 under a 12-h light/dark cycle with free access to food and water for 1 week before use. Sprague Dawley rats (males, 276-300 g, n=4) were purchased from Charles River Laboratories (Wilmington, Mass.). Pharmacokinetic studies were conducted to evaluate the pharmacokinetic profiles of LPV and RTV from LPV/RTV ISNPs. Rats were treated with 100 ul of LPV/RTV ISNPs to provide 100 mg/kg of LPV by subcutaneous injection. After the injection, the inventor collected the blood at 0, 1, 2, 3, 5, 6, 24 hours and 2, 3, 4, 5, 6 days. The blood was immediately centrifuged at 3400 rpm for 5 min at 4° C., and 100 ul of plasma samples were transferred to a new tube and stored at −20° C. until further analysis within 3 weeks. LPV and RTV concentrations in the plasma were determined by liquid chromatography-mass spectrometry (LC-MS, an Agilent G6460 Triple Quad LC-MS system) using a previously reported method.

B. Results

Characterization of LPV/RTV ISNPs.

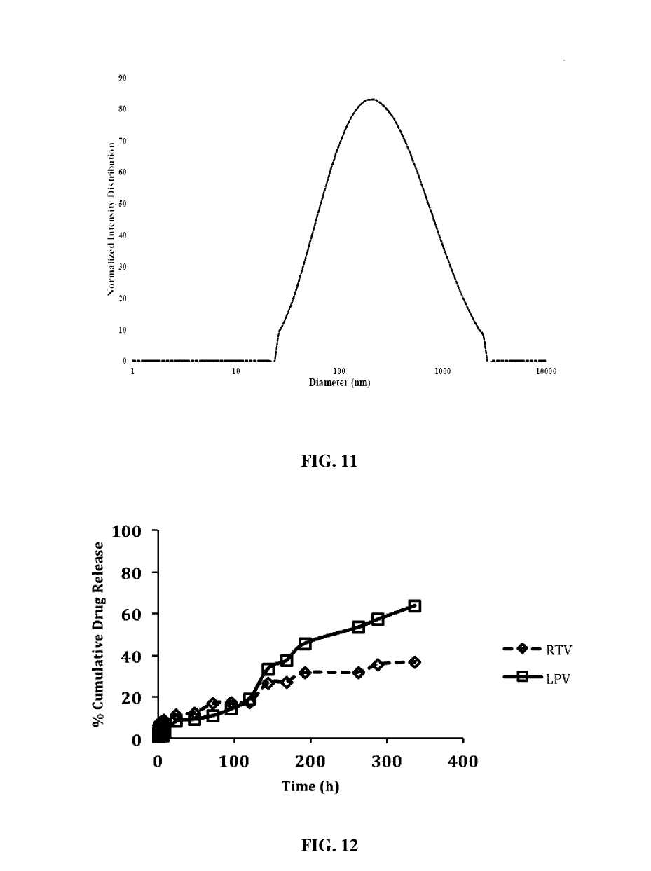

As shown in Table 10, particle size of LPV/RTV ISNPs were 167.8±9.9 nm (n=3) after the inventor reconstituted the pro-NP with 0.9% NaCl. LPV/RTV ISNPs were monodispersed with a narrow size distribution (P.I.<0.35) (FIG. 11). Particle sizes were consistent with the result from LPV/RTV ISNP granules in the inventor's previous study. As shown in Table 1, the inventor was able to entrap 98.9% LPV and 98.7% RTV in the ISNP granules after reconstitution, respectively. The drug loadings were 23.5% LPV and 5.9% RTV, respectively.

TABLE 10
Physico-chemical properties of LPV/RTV ISNPs.

| | | | | | DL % |
|---|---|---|---|---|---|
| | Mass | Particle | | | (w/w, |
| Drugs | (μg/ml) | size^{a} (nm)^{#} | P.I. | EE % | drug/oil) |
| LPV | 25 | 167.8 ± 9.9 | 0.310 ± 0.024 | 98.9 ± 0.5 | 23.5 |
| RTV | 6.25 | | | 98.7 ± 0.2 | 5.9 |

In Vitro Release Studies.

The in vitro release profile of LPV/RTV ISNPs was investigated in PBS buffer in a physiological condition (pH 7.4). The cumulative LPV and RTV release from LPV/RTV ISNPs is shown in FIG. 12. LPV and RTV exhibited the sustained release profiles. A slow release rate of LPV was observed about 20% on 5 days for LPV, followed by a sustained release beyond 14 days. RTV releases faster than LPV in the first 5 days but slower than LPV after that. The inventor's ISNP formulation did not showed any burst release patterns, confirming that drugs were homogenously dispersed with excipients. This proven that LPV and RTV were loaded inside oleic acid and TPGS and that the drugs were not on the surface of the ISNPs.

Pharmacokinetics.

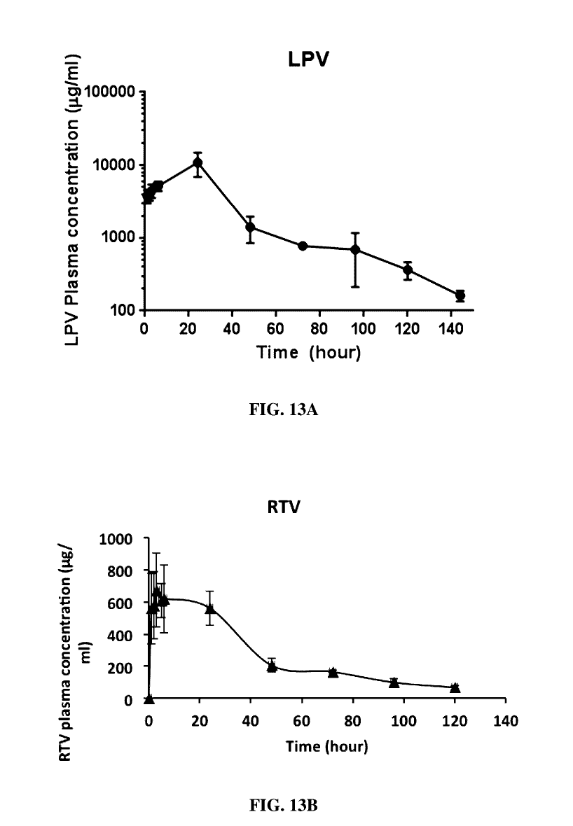

Plasma concentrations of LPV and RTV are plotted in FIGS. 13 A-B. C_{trough} is the lowest drug concentration during the course of a dosing interval, typically occurring immediately prior to the next scheduled dose. After 6 days, LPV C_{trough} remained above 160 ng/ml, and RTV C_{trough} was about 50 ng/ml with one subcutaneous injection. In the injection sites, the inventor did not observe any irritation and inflammation. Mice did not show any discomfort during the treatments.

C. Discussion

Lipid NPs are the ideal technologies for poorly water-soluble drugs. Several methods had been done to prepare lipid-based NPs. However, these NPs are prepared in a liquid form, and consequently long-term storage in solution and stability of nanoparticle sizes are problematic. To solve the issues, techniques, such as freeze-drying and spray-drying (Li et al., 2011; Ohsima et al., 2009), are used to dry the nanoparticles. The disadvantages of these approaches are time consuming and size increase after reconstituting the dyed nanoparticles. The novel ISNP technology offers the novel procedure to prepare nanoparticles without using water. Pro-NP (a mixture of lipid and surfactant) spontaneously forms the ISNP with appropriate particle size and distribution when in contact with saline. Therefore, the ISNP technology overcomes the aforementioned issues associated with other lipid-based nanoparticles. For example, the stability of particle size is less concerned for the ISNPs because pro-NP is the final formulation kept on the shelf and the ISNPs form dafter adding saline. The preparation procedure is very simple. The excipients, oleic acid and TPGS, are natural materials and have been approved by the FDA. Thus, the novel ISNP technology has a great potential to produce parenteral nanoformulations for clinical translation.

Development of FDC nanoformulations for injection is quite challenging. Drugs in the FDC have to be encapsulated into one nanoparticle. Very often, the nanoparticle could be suitable for one drug, but not for the other drug, because drugs have different physicochemical properties. LPV and RTV have been encapsulated into PLGA nanoparticles; however, less than 46% of LPV and RTV were entrapped into the nanoparticles (Destache et al., 2009). With the ISNP technology, drugs are dissolved in the excipients, and then encapsulated through a self-assembly process by binding to the excipients to form the ISNPs. This novel mechanism leads to the minimum amounts of excipients with higher EE % and drug loading. In this study, the inventor successfully entrapped over 98% of LPV and RTV into one nanoparticle with 23.5% drug loading for LPV and 5.9% drug loading for RTV by using the ISNP nanotechnology. The new LPV/RTV ISNPs could be a potential FDC for injection to treat HIV.

In addition to the aforementioned advantages, the inventor demonstrated the sustain-released properties of drug-loaded ISNPs in this study. According to the in vitro release, about 60% LPV and 40% RTV were released from the ISNPs after 14 days. The further animal pharmacokinetic study confirmed that the ISNPs maintained the drug concentrations over 6 days by one subcutaneous injection. Because of the limited fluid volume in the injection site, the ISNPs could form a “depot” and slowly released the drugs over time. Long-acting injectable formulations offer alternative therapeutic options for the treatment of HIV. Nanoparticulate drugs through subcutaneous injection can retain in the lymphatic system and improve exposure of the drugs in intracellular and overcome HIV lymphatic drug insufficiency as by oral dosing (Spreen et al., 2013; Ho et al., 2015). Additionally, LPV/RTV are both lipophilic molecules that move easily in lipoidic compartment in underlying subcutaneous tissue (Schneider et al., 2009); thus, LPV/RTV ISNP injection will facilitate the drug movement through the lymphatic circulation. The inventor did not observe any redness, swelling, irritation on the mouse skin, suggesting that LPV/RTV ISNPs did not cause skin hypersensivity reaction and trigger immune response. LPV/RTV ISNP injection could be advantageous in term of improving patient adherence and lowering dose medication on the next dosing schedule and helping in management of HIV.

To conclude, in this study LPV/RTV ISNPs were successfully prepared for the purpose of a long-acting injection by using the novel ISNP nanotechnology. After mixing saline with LPV/RTV pro-NP, LPV/RTV ISNPs formed with particle size of 168 nm and over 98% of EE % for both drugs. The preparation of the ISNPs is simple and scalable with few concerns on nanoparticle stability. The LPV/RTV ISNPs exhibited the sustained release behavior after subcutaneous injection into mice as well as in an in vitro physiological condition. Therefore, the novel ISNP nanotechnology has great potential to produce long-acting FDC injectable nanoformulations. LPV/RTV ISNPs could be new nanoformulation to improve patient adherence and therapeutic effectiveness.

### Example 6

Prostate cancer (PCa) is the most common malignancy in men and the second leading cause of cancer death after lung cancer among men in the United States (Siegel et al., 2014). Androgen deprivation therapy (ADT) is used as a standard treatment for metastatic prostate cancer. The response rate of prostate cancer to ADT can be up to 80% with monotherapy and more than 90% with combination therapy (Suzuki et al., 2007). Despite to this response rate, most prostate cancers eventually stop responding to this treatment referred to as castration resistant prostate cancer (CRPC).

Docetaxel (DTX)-based therapy remains the mainstay of treatment options with CRPC and has shown improving overall survival (Petrylak, 2006). DTX is a semisynthetic compound belonging to the taxane family found in the European yew tree Taxus baccata (Bissery et al., 1991). It is a microtubule stabilizing agent that promotes tubulin assembly and stabilizes the polymers against depolymerization (Ringel and Horwitz, 1991). While DTX shows promising outcomes, it is not the ultimate solution for patients with CRPC since all men eventually develop resistance to a drug or unable to tolerate its toxicities and adverse events (Berthold et al., 2008; Singer and Srinivasan, 2012 and Hennenfent and Govindan, 2006). DTX is highly lipophilic with poor water solubility that requires a complex solvent system for its commercial formulation (Hennenfent and Govindan, 2006). Current formulation of DTX with Tween 80 may cause hypersensitivity reactions due to its water insolubility and reduce uptake by tumor tissue (Weiszhar et al., 2012 and Ma et al., 2011). To avoid these disadvantages, a new DTX formulation in the absence of Tween 80 is necessary. In addition, a potential resistance mechanism for CRPC includes drug efflux pump, microtubule alterations, and apoptotic defects (Antonaraski and Armstrong, 2011). The most extensively studied mechanism of resistance is the overexpression of P-glycoprotein (P-gp) and multidrug resistance protein 1 (MDR-1) (O'Neill et al., 2011 and Kawai et al., 2000). P-gp and MDR-1 are membrane proteins belonging to the ATP-binding cassette (ABC) family of transporters. It has been shown to pump substrates, including DTX, out of tumor cells through an ATP-dependent mechanism, therefore reduce intracellular concentrations of a drug.

Nanoparticle (NP) drug delivery systems offer alternative therapeutic options for the treatment of PCa that have the potential to control release of drugs, alter and improve the pharmacokinetic and pharmacodynamic properties of drug molecules, protect drugs from degradation, and reduce toxicities and side effects (Jong et al., 2008 and Markman et al., 2013). NPs expect to increase the concentration of drugs in cancer cells due to enhanced permeation and retention (EPR) effect and to avoid chemotherapy toxicity in normal cells (Dong and Mumper, 2010). Furthermore, the use of NPs may overcome or at least reduce drug resistance. Currently, lipid-based nanoformulations for DTX included liposomes, micelles, solid lipid NPs, and nanocapsules have been developed to solubilize lipophilic and poor water solubility drug, resulting in enhanced the efficacy and bioavailability of the drug (Zhang et al., 2014; Ostacolo et al., 2014; Mosallaei et al., 2013 and Nassar et al., 2011). To date, a study has reported that DTX lipid NPs comprised of DTX conjugated various fatty chain lengths showed to lower cytotoxicity compared to free DTX in the sensitive prostate cancer cell lines but did not target the study on resistant prostate cancer cell lines (Feng et al., 2011).

In this study, the inventor developed a novel lipid-based DTX NPs, named as DTX MT NPs, and evaluated cytotoxicity of DTX MT NPs in sensitive and resistant prostate cancer cell lines, PC-3 and DU145. DTX MT NPs were prepared by an oil-in-water (o/w) emulsion method. D-α-tocopheryl polyethylene glycol 1000 succinate (TPGS) was used as the drug surfactant and Miglyol 812 was used as the oil phase. Miglyol 812 is a biocompatible/biodegradable, medium-chain triglyceride (Guo et al., 2013). TPGS as a water-soluble vitamin E drug delivery vehicle is approved by the Food and Drug Administration (FDA). It has many potential applications including P-gp inhibitor, solubilizer, and permeation enhancer (Dong et al., 2009). Therefore, the blank NPs are safe. In addition, DTX MT NPs were characterized in terms of particle size, zeta potential, polydispersity index (PI), drug entrapment efficiency (EE), drug loading (DL), in vitro release study, and stability studies.

A. Materials and Methods

Materials and Cell Culture.

Docetaxel was purchased from LC Laboratories (Woburn, Mass.). TPGS, Kollisolv® MCT70, Kolliwax® GMSI, Tween 80, Tween 20, and Poloxamer 188 were obtained from BASF (Florham Park, N.J.). Migloyl 812 and Imwitor® 960 were obtained from Cremer (Eatontown, N.J.). Oleic acid was obtained from Spectrum (Gardena, Calif.). Labrafac was purchased from Gattefosse (Cedex, France). Microcon Y-100 with MWCO 100 kDa was purchased from Millipore (Bedford, Mass.). Ethanol (200 proof, USP grade) was purchased from Pharmco-AAPER (Brookfield, Conn.). Methanol and acetonitrile (HPLC grade) were purchased from Fisher Scientific (Pittsburgh, Pa.). Phosphoric acid (85 wt %) and dimethyl sulfoxide (DMSO) were purchased from Sigma-Aldrich (St. Louis, Mo.). PBS and RPMI 1640 medium were purchased from ATCC (Manassas, Va.). The sensitive prostate cancer cell lines, PC3 and DU145, were obtained from ATCC. The resistant prostate cancer cell lines, PC and DU145, were obtained from the University of Michigan. Cells were maintained in RPMI 1640 supplemented with 10% fetal bovine serum (FBS) and 1% penicillin-streptomycin. Cells were cultured at 37° C. in humidified incubator with 5% CO₂ and maintained in exponential growth phase by periodic subcultivation. Resistant cell lines were treated with 5 nM docetaxel in DMSO.

Screening of Oils and Surfactants.

These studies were performed to test the solubility in oils and surfactants. The solubility of DTX was determined in oils (oleic acid, Migloyl 812, Kollisolv® MCT70, Kolliwax® GMSI, Imwitor® 960, and Labrafac) and surfactants (TPGS, Poloxamer 188, Tween 20, and Tween 80). Oils and surfactants were accurately weighed into glass vials. Excess amounts of DTX were added into glass vials containing vehicles. The vials were stirred on the hot plate maintained at 65° C. for 3 min. The drug solubility (w/w) was calculated.

Preparation of DTX NPs.

NPs were prepared by a warm o/w emulsion method as describe in Example 1. Defined amounts of Migloyl 812 and TPGS with a 1:1 (w/w) ratio were weighed into glass vials and heated to 35° C. Two (2) mL of preheated Milli-Q water to 65° C. were added into the mixture of melted oil and surfactant. The mixture was stirred at 1,300 rpm for 20 min with stirring at room temperature to allow it to cool down. To prepare DTX MT NPs, 75 μL, of 4 mg/mL DTX dissolved in ethanol was added directly to the melted oil and surfactant. Ethanol was removed by N₂ stream prior to initiating the process described above. Particle size and size distribution of NPs were measured using a N5 Submicron Particle Size Analyzer (Beckman Coulter). Ten microliters of prepared NPs were diluted with 1 mL of Milli-Q water to reach within density range required by the instrument, and particle size and analysis was performed at 90° light scattering at 25° C.

Physiochemical Characterization of DTX NPs. Particle Size and Zeta Potential Measurement.

Ten microliter of blank NPs and DTX MT NPs were diluted with 1 mL of Milli-Q water and added 10 μL of PBS buffer (pH 7.4) for measurement of zeta potentials using a Delsa Nano C Particle Size Analyzer (Beckman Coulter Inc., CA, USA)

Determination of Drug Loading and Entrapment Efficiency.

The concentration of DTX MT NPs was quantified by HPLC using Inertsil ODS-3 column (4.6 mm×150 mm, 5 μm particle size, GL Sciences Inc, CA, USA). The chromatography conditions were as follows: the mobile phase consisted of 0.1% ortho phosphoric acid and acetonitrile (40:60, v/v) at a flow rate of 1.0 mL/min, and the effluent was detected at 230 nm. The mobile phases were degassed before use. The injection volume was 30 μL, and the analysis time was 4.9 min per sample. The retention time of DTX was about 4.9 min. For DTX standard curve, DTX was dissolved in methanol. The curve was found to be linear in the concentration range 0.5-100 μg/mL and r=1. To quantify DTX in NPs, 1 part of DTX MT NPs in water were dissolved in 8 parts of methanol. Drug loading and entrapment efficiencies were determined by separating free DTX and DTX-loaded NPs using Microcon Y-100, and then measuring DTX NP-containing supernatants. To ensure mass balance, the filtrates were also assayed for DTX. The drug-loading content and drug entrapment efficiency were calculated based on the following formula:

$$\% drug loading = \frac{drug entrapped in NPs}{weight of oil} 100 \% ( w \text{/} w )$$

$$\% drug entrapment efficiency = \frac{drug entrapped in NPs}{total drug added into NP preparation} 100 \% ( w \text{/} w )$$

Particle size stability of NPs in 4° C. and 37° C.

The physical stability of DTX NP suspension was evaluated over storage at 4° C. for six months. The stability of all NP suspensions was also analyzed at 37° C. in 10 mM PBS, pH 7.4 by adding 100 μL NP suspension to 13 mL PBS buffer with a water-bath shaker mixing at 150 rpm. At each time interval, 1 mL aliquots were removed and allowed to equilibrate to room temperature before particle size measurements.

In Vitro Release Study.

DTX release studies (n=3) were completed at 37° C. using PBS buffer as release medium. 200 μL, of DTX MT NPs was added into 20 mL 1×PBS and shaken at speed of 135 rpm at 37° C. 500 μL from previous sample was added into centrifugal filter and centrifuged at 14,000 rpm. 200 μL of methanol was added into centrifugal filter and performed a reverse spinning to collect DTX MT NPs. The solution was then vortex and transferred into the insert of HPLC vial. The concentration of DTX released from the NPs was determined by using HPLC assay.

In Vitro Cytotoxicity-MTT Assay.

The cytotoxicity of DTX MT NPs was tested in PC3 and DU145 sensitive and resistant cell using MTT [3-(4,5-dimethylthiazol-2-yl)-2,5-diphenyltetrazolium bromide] assay. Cells were seeded into 96-well plates with a density of 7,000 cells/well and incubated at 37° C. for 24 hr under 5% CO₂. Following that, cells were treated with a drug equivalent concentration ranging from 0.5-10,000 nM for DTX, DTX MT NPs, and blank NPs. Due to low water solubility, DTX was dissolved with DMSO. After 72 hr of incubation, the medium was removed from each well and replaced with 90 μL of DMEM medium without phenol red and 10 μL of MTT (5 mg/mL in PBS). The plates were incubated at 37° C. with 5% CO₂ for 4 hr. Formazan produced by mitochondria enzymes of living cells was dissolved in 150 μL of isopropanol containing 0.04 N HCl for 5 min. The absorbance was measured at a test wavelength of 570 nm and a reference wavelength of 650 nm in a microplate reader (Synergy H1 hybrid Multi-Mode Microplate Reader)

Statistical Analysis.

Statistical comparisons were made with ANOVA followed by pairwise comparison using Student's t test using GraphPad Prism software. Results were considered significant at p<0.05.

B. Results

Preparation of DTX MT NPs.

To avoid safety issues related to the excipients, the inventor chose FDA-approved excipients for this study. The solubility of DTX in various oils and surfactants at 65° C. was determined. As shown in Table 11, DTX was found to have the highest solubility in Migloyl 812 for oil and Tween 80 for drug surfactant. Therefore, Migloyl 812 was chosen for further preparation of lipid-based NPs. Tweens and TPGS are nonionic surfactant that can enhance solubility of poorly water soluble drug. However, the use of Tween 80 in DTX formulation can cause adverse events. In addition, TPGS has the ability to suppress multi-drug resistance (MDR) by inhibiting P-gp activity, therefore TPGS was chosen over Poloxamer 188 and Tweens (Zhu et al., 2014).

Particle Size and Zeta Potential.

The mean particle sizes and PI of all tested NPs are shown in Table 12. All NPs had mean particle size diameters of less than 160 nm and PI of less than 0.32. These results suggested that the entrapment of DTX had no influence on the mean particle size of DTX MT NPs. Negative zeta potential value was observed for blank NPs and DTX MT NPs with zeta potentials of −39.1 to −45.5 mV, respectively. Zeta potential gives an indication of the potential stability of the suspension and indicates the degree of repulsion between adjacent, similarly charged particles in a suspension. These results indicated that zeta potential value small than −30 mV played an important role to prevent aggregation of NPs in PBS buffer.

Drug Loading and Entrapment Efficiency.

The lipid-based NPs offer a highly versatile delivery drug platform to carry hydrophobic drugs with high loading yields and high entrapment efficiencies. The lipid core can accommodate large amount of hydrophobic drugs that can be encapsulated either inside lipid core or chemically linked to TPGS. As shown in Table 12, the DL and EE for DTX MT NPs were 3% and 91%, respectively. High EE might be due to the tendency of DTX as a hydrophobic molecule to enter Migloyl 812 as a hydrophobic core of the NPs.

Physical Stability of NPs.

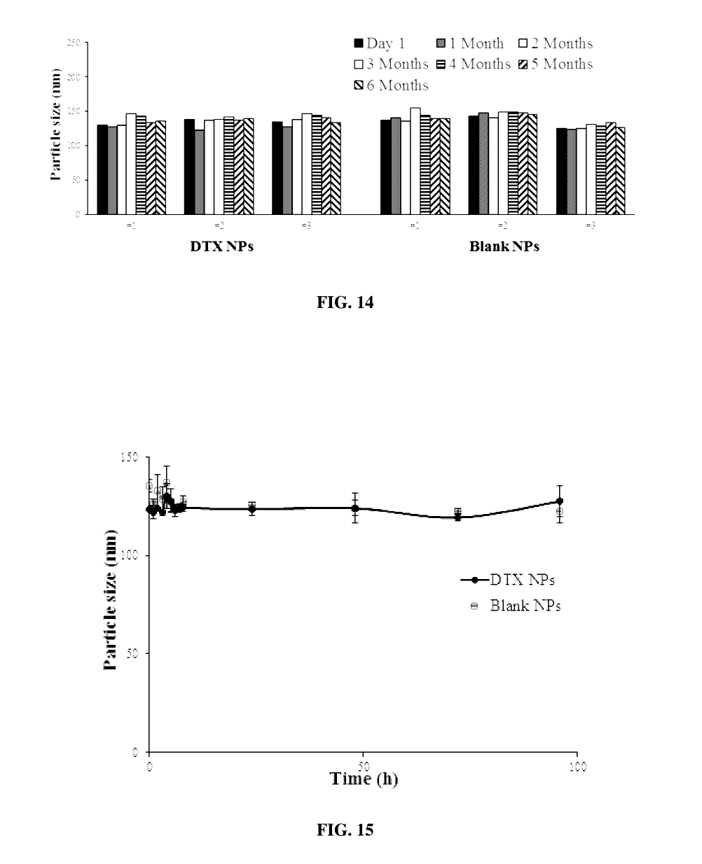

The physical stability of DTX MT NPs was investigated by monitoring the changes in particle sizes at 4° C. for long-term storage and at 37° C. for 96 hr in PBS to mimic physiological conditions. The NPs with and without DTX stored as liquid suspensions did not show significantly changes in particle sizes at 4° C. during six-month storage (FIG. 14). Under physiological conditions, particle sizes for DTX-loaded and blank NPs remained stable for 96 hr (FIG. 15).

In Vitro Release Study.

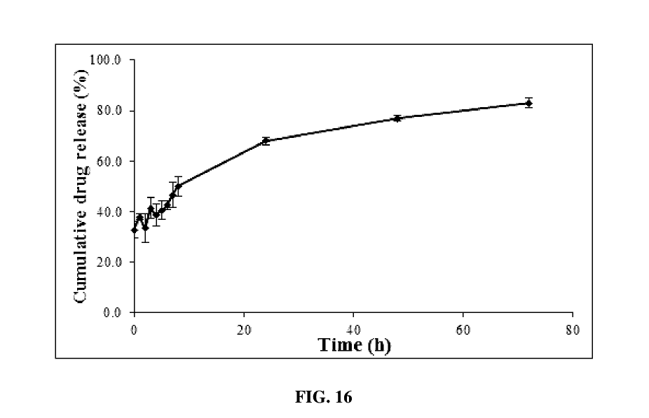

Drug release study of DTX MT NPs in PBS buffer at 37° C. appeared to be released in a biphasic pattern, with an initial burst release and followed by a sustained release (FIG. 16). Around 30% of the drug was released at the initial time point and followed in continuous way for up to 72 hours reaching 80% of cumulative release.

In Vitro Cytotoxicity Studies.

The cytotoxicity of DTX and DTX MT NPs in sensitive and resistant prostate cancer cell lines, DU145 and PC3, was evaluated using MTT assay (Table 13). Blank NPs showed no toxicity in all tested cell lines (IC₅₀, >2 μM). In sensitive cell lines, the IC₅₀ values of DTX MT NPs were no significant difference from those of free DTX. Both free DTX and DTX MT NPs in PC3 and DU145 resistant cell lines showed higher than in IC₅₀ values when compared to PC3 and DU145 sensitive cell lines. However, DTX OT NPs induced the greater cytotoxicity in the resistant DU145 cells (p<0.03) and PC3 cells (p<0.002) compared to free DTX. As shown in Table 11, the IC₅₀ value of DTX MT NPs in resistant PC3 cell line was 1.5-fold lower than that of free DTX, and the IC₅₀ value of DTX MT NPs in resistant DU145 cells was 1.8-fold lower than that of free DTX.

C. Discussion

Docetaxel remains the standard first line therapy for CRPC. However, as previously noted, the commercial formulation of DTX, Taxotere®, is formulated using Tween 80 and ethanol (50:50, v/v) that can cause adverse events including hypersensitivity reactions. Despite that patients received pre-treatment with corticosteroids prior using DTX, hypersensitivity reactions with DTX can still occur (Norris et al., 2010). An ideal formulation should avoid the risk associated with the surfactant Tween 80 related anaphylactic fatalities. In addition, the ability to create drug delivery systems to overcome biological barriers, deliver hydrophobic, poorly water-soluble drugs, and selectively allow drugs to release at target site at a therapeutic concentration are desired (Desai, 2012). Lipid-based drug delivery systems have been developed to overcome these problems (Kroon et al., 2014 and Jin et al., 2014).

In the present study, the inventor were successfully developed DTX MT NPs. To avoid toxicity associated with excipients, the FDA-approved pharmaceutical excipients were screened to test the solubility of DTX. In terms of solubility screening, Migloyl 812 as the oil phase and TPGS as the surfactant phase were chosen. Migloyl 812 is a lipid at room temperature that helps to solubilize poorly water-soluble drugs so that drugs remain dissolved inside oil core and thus may increase drug payload (Dong et al., 2009 and Hippalgaonkar et al., 2010). TPGS was selected for this study to emulsify and solubilize DTX and also to inhibit P-gp. With this design, the EE % of DTX was very high in the MT NPs with an appropriate drug loading. The particle size of NPs plays a critical parameter that can affect the physical stability, biodistribution and drug release from NPs, and cellular uptake (Cho et al., 2013). It was previously reported that the CRPC shows hypervascular permeability and impaired lymphatic drainage. This property allows NPs to have a higher probability to extravasate from the vascular compartment into the tumor interstitium and to decrease the clearance of high-molecular-weight compound from the tumor interstitium (Luo et al., 2010). Moreover, NPs with certain particle size tend to accumulate in tumor tissues better than normal tissues through the EPR effect, which is referred to as size-dependent passive targeting. Studies have been suggested that particles between 100 to 200 nm are desired for adhesion to and interaction with cells. It can also enhance intracellular uptake and retention effect of NPs (Kulkami and Feng, 2013 and Cho et al., 2008). These results showed that all MT NP batches have the mean particle size within this range (<160 nm), which is ideal to take advantages of NPs for cancer drug delivery (Table 10). Particles with zeta potential value larger than +30 mV or smaller than −30 mV are normally considered stable for colloidal dispersions (Mitri et al., 2011). The zeta potentials of the MT NPs were found to be in the size range of −39.1 to −45.4 mV, indicating good dispersion stability. Indeed, stability measurements demonstrated that DTX MT NPs were stable over 6 months at 4° C. (FIGS. 6A-B) and over 96 hr under a mimicked physiological condition (FIG. 7). An initial burst release was observed for DTX MT NPs, which might associate with the distribution of DTX on the surface of the NPs. The location of the drug on the surface of NPs permits rapid release when it comes in contact with surrounding fluid. The remaining portion was located within the lipid core of the NPs and could release slowly over 72 hours.

Next, the inventor investigated the cytotoxic effect of DTX MT NPs in sensitive and resistant CRPC cell lines, PC3 and DU145. TPGS has been reported to improve the adhesion of NPs to cancer cells, overcome multidrug resistance, and inhibit P-gp (Duhem et al., 2014 and Koudelka et al., 2015). DTX MT NPs had the same cytotoxicity with free DTX in sensitive cell lines (p>0.05). The IC₅₀s for the blank MT NPs were over 2500 nM (Table 11), indicating that the blank MT NPs were very safe for these cell lines. By carefully choose the excipients for NP development, the inventor prevented the cytotoxicity from the formulation vehicles as well as keeping the cytotoxicity of the drug; thus, DTX MT NPs could be an appropriate alliterative to the currently commercial DTX formulation with reduced side effects associated with the excipients. This result is different from previously reported with DTX-lipid conjugated NPs with different fatty acid chain lengths in which free DTX had higher cytotoxicity DTX conjugates in sensitive DU145 cells (Dong et al., 2009). Takeda et al. demonstrated that P-gp and MDR-1 mRNA were responsible for drug resistance in DU145 and PC3 resistant cells (Mizokami et al., 2007). Moreover, the level of P-gp in DU145 resistant cells was more expressed than that in PC3 resistant cells. Overexpression of P-gp is a major factor in resistant DU145 cells, but not in PC3 cells; other resistant mechanisms, such as microtublin alteration and changes of apoptosis genes, were mainly identified in PCs resistant cells. Compared to the IC₅₀s of free DTX, DTX MT NPs significantly improved potency of DTX in both resistant cell lines, demonstrating that DTX MT NPs overcame drug resistance in both PC3 and DU145 cell lines. Drug-loaded NPs have been studied for P-gp-mediated drug resistance. It is also known that TPGS is P-gp inhibitor. Thus, the inventor expected to see DTX MT NPs to reduce the IC₅₀ of DTX in resistant DU145 cells. However, very few studies discussed NPs for drug resistance that is not controlled by P-gp overexpression, especially in CRPC. The finding that DTX MT NPs overcame drug resistance in PC3 cells is very interested.

In this study, DTX MT NPs were successfully prepared using an oil-in-water emulsion method. DTX MT NPs has particle size of 114 nm with a narrow size distribution and over 91% of EE %. They were stable at 4° C. over 6 months. DTX MT NPs showed same cytotoxicity in sensitive prostate cancer cells and superior cytotoxicity in resistant prostate cancer cells compared to free DTX. DTX MT NPs could be considered as a promising alternative to the commercial DTX formulation to avoid toxicity related to the excipients, and also could be the second-line treatment for DTX resistant CRPC.

TABLE 11
Solubility of docetaxel in oils and surfactants

| Oil | Solubility (w/w) | Surfactant | Solubility (w/w) |
|---|---|---|---|
| Oleic acid | 0.066 | TPGS | 0.008 |
| Migloyl 812 | 0.079 | Poloxamer 188 | 0.019 |
| Kollisolv MCT70 | 0.038 | Tween 20 | 0.052 |
| Kolliwax GMS I | 0.025 | Tween 80 | 0.082 |
| Imwitor 960K | 0.053 | | |
| Labrafac | 0.022 | | |

TABLE 12
Physiochemical properties of blank and docetaxel NPs

| | Theoretical | Mean | | Zeta | DL (% | |
|---|---|---|---|---|---|---|
| | loading | diameter | | potential | w/w, | |
| Formulations | (μg/mL) | (nm) | PI | (mV) | drug/oil) | EE (%) |
| Blank NPs | n/a | 134.9 ± 9.3 | 0.237 ± 0.006 | −39.1 ± 0.7 | n/a | n/a |
| DTX MT NPs | 150 | 111.4 ± 9.5 | 0.225 ± 0.014 | −45.4 ± 2.1 | 3.44 | 91.6 ± 0.7 |

Data are shown as mean ± SD, obtained from three batches.
PI, polydispersity index;
DL, drug loading;
EE, drug entrapment efficiency

TABLE 13
Docetaxel (DTX) cytotoxicity studies at 72 hr

| | DTX (nM) | DTX NPs (nM) | Blank NPs (nM) |
|---|---|---|---|
| PC3 sensitive | 7.0 ± 0.4^{#} | 7.4 ± 0.1^{#} | 2563.0 ± 149.1 |
| DU145 | 6.8 ± 0.5^{##} | 7.2 ± 0.3^{##} | 2767.0 ± 180.0 |
| sensitive | | | |
| PC3 resistance | 75.3 ± 2.7* | 50.2 ± 2.3* | 2837.0 ± 619.9 |
| DU145 | 870.3 ± 85.4** | 493.2 ± 53.8** | 5559 ± 681.3 |
| resistance | | | |

IC₅₀ values (nM) of DTX, DTX MT NPs, and blank NPs in DU145 and PC3 sensitive cell lines and DU145 and PC3 resistant cell lines.
Data are presented as the mean ± SD of triplicate measurements (n = 3).
^{#}, ^{##}p > 0.05 and *, **p < 0.05.

### Example 7

Prostate cancer is the second leading cause of death in men in the United States. It represents one of the major epidemiological problems in older men (Sanna and Sechi, Maturitas, 2012). Anti-androgen is the first line treatment of patients who diagnosed with prostate cancer (PCa), but eventually most patient will develop androgen-independent resistant in prostate cancer that are highly metastatic which referred to as castration-resistant prostate cancer (CRPC) (Van Brussel and Mickisch, 2003). Taxanes (Docetaxel and Paclitaxel) appeared to be the most efficacy drugs for the treatment of CRPC (Tannock et al., 2004); however, eventually patients develop DTX resistance. Predominantly, the cancer-related mortality stems from the development of DTX resistance. Multiple mechanisms exist for drug resistance and a drug is not confronted for a single mechanism. Resistance to Taxanes is associated with overexpression of P-gp, the product of multiple drug resistant (MDR1) that can efflux the drugs out (Abidi and Cabazitaxel, 2013). The fairly compelling data repeatedly showed the correlation between P-gp overexpression and poor clinical outcomes in many cancers including PCa (Wilken et al., 2011 and Anand et al., 2007). Indeed, P-gp overexpression is the main cause of resistance in paclitaxel-resistant DU145 cells (DU145-TxR). However, the use of P-gp inhibitors to enhance efficacy in drug resistance cancers has not been successful in clinical trials (Shishodia et al., 2007; Anand et al., 2007 and Anand et al., 2008). Because of the lack of specificity, P-gp inhibitors also inhibit P-gp in normal tissues, leading to significant side effects. Although new cytotoxicity agents, e.g. cabazitaxel, were developed to avoid P-gp recognition, tumor heterogeneity still generates drug resistance. Recently, it has been demonstrated that up-regulation of apoptosis genes and microtublin alterations are also resistant mechanisms in CRPC^{[5,6]}. Thus, a new agent is needed to select for overcoming the resistance mechanisms of CRPC.

Certain dietary supplement known as phytochemical plays an important role in suppression of tumor cell proliferation and prevents adverse effects from commonly related to current chemotherapies (Mates et al., 2011). Curcumin is a promising cancer chemopreventive agent and has been extensively studied over the past decade. Curcumin is a bioactive compound extracted from the rhizome of the Curcuma longa plant (Ratz-Lyko et al., 2014). It has a low molecular weight and is a yellow, lipophilic polyphenolic compound of Indian spice turmeric (Mishra and Palanivelu, 2008). Due to a wide range of biological and pharmacological activities, curcumin contains anti-inflammatory and antioxidant properties (Aggarwal and Shishodia, 2006; Aggarwal and Sung, 2009 and Shishodia et al., 2007). Moreover, curcumin has shown to be the most effective in inhibitory effects by induced cell cycle arrest and apoptosis in numerous human cancer cell lines (Wilken et al., 2011 and Anand et al., 2007). The inhibition of carcinogenesis by CUR is influenced by different mechanisms such as nuclear factor-kappaB (NF-kB), transcription factor activator protein-1 (AP-1), mitogen activated protein kinase (MAPK), tumor protein 53 (p53), nuclear β-catenin signaling, and AKT signal pathways (Anand et al., 2008; Hatcher et al., 2008 and Parveen and Sahoo, 2008). Deeb et al. demonstrated that CUR was able to block phosphorylation of Ikβα and inactivated NF-kB. Thus, CUR can increase sensitivity of prostate cancer cells in PC-3, DU145 and LNCaP (Deeb et al., 2004). In addition, curcumin is known to downregulate the three transporters including P-glycoprotein (P-gp), multidrug resistance associated protein (MRP1), and breast cancer resistance protein (ABCG2) (Limtrakul et al., 2007 Chearwae et al., 2006. Despite its superior properties, curcumin has been limited in use in clinical trial due to hydrophobicity, low water solubility, instability, and poor pharmacokinetics (Wang et al., 1997; Tonnesen et al., 2002 and Shoba et al., 1998). Drug delivery methods need to be investigate to overcome the drawback of delivering CUR.

NPs have the potential to improve the therapeutic index of currently available drugs by increasing drug efficacy and achieving steady state therapeutic levels of drugs over time. The effectiveness of cancer therapy in solid tumors depends on adequate delivery of the therapeutic agents to tumor cells. NPs can pass through leaky and hyperpermeable tumor vasculature and accumulate in the tumor vicinity utilizing the enhanced permeability and retention (EPR) effect. Further, actively targeting delivery is afforded by the use of ligand-coated NPs that bind to a cancer cell receptor, resulting in cell-specific recognition to improve drug delivery for solid tumors. Several different nanoparticle-based formulations of CUR were extensively studied including solid dispersion nanoparticles, liposomes, and polymeric nanoparticles (Grama et al., 2011 and Verderio et al., 2013). These formulations had drawbacks due to drug leakage, poor physical stability, and toxicity of excipients (Sun et al., 2013). Dhule et al. prepared liposome coated with CUR and cyclodextrins for treatment of osteosarcoma and reported disadvantages such as low stability and encapsulation efficiency (Popat et al., 2014). Mazzarino et al. prepared CUR encapsulated with chitosan nanoparticles and coated with polycaprolactone; however, there was no release study nor cellular uptake (Mazzarino, 2012). Thus, lipid-based nanoparticles (LNPs) delivery system is an emerging alternate system in promising approach in overcome disadvantages of CUR. This system has several advantages due to biodegradable and/or biocompatible nature of lipids, resulting in low toxicity. The lipid-based system typically composed of excipient combination consisting of oil, surfactant, and water. D-alpha-tocopheryl poly(ethylene glycol) succinate 1000 (TPGS) is widely used as a stabilizer and biocompatible. It inhibits drug efflux through allosteric regulation of P-gp ATP enzyme (Collnot et al., 2010). Further, TPGS has been approved by the US Food and Drug Administration (FDA) as safe pharmaceutic excipient and has been cooperated into many nanoparticle formulations. Migloyl 812, also FDA-approved, was selected as oil phase due to biodegradable and biocompatible properties, to facilitate the formation of 0/W phase (Dong et al., 2009).

In the previous studies, the inventor has developed paclitaxel (PX) and doxorubicin-loaded NPs (named BTM NPs) to overcome P-gp-mediated drug resistance in cancer (Abidi and Cabazitaxel, 2013 and Chun and Lee, 2004). The in vitro studies demonstrated that PX BTM NPs can overcome P-gp-mediated multidrug resistance by endocytosis, P-gp inhibition, ATP depletion and mitochondria alteration. Moreover, the study showed that the pharmaceutical excipients and the empty BTM NPs are P-gp inhibitors. Of interest, the mixture of the empty NPs and PX did not inhibit tumor growth while PX BTM NPs showed marked anti-cancer efficacy in the mice bearing resistant cancer. These results emphasize the importance of coincidentally delivering chemosensitizers and anti-cancer drugs. The objective of this study is to develop novel CUR MT NPs as a therapeutic agent to treat resistant CRPC. The inventor developed CUR MT NPs, so called CUR MT NPs, by optimizing NPs with varying lipids and surfactant and tested them in sensitive and resistant DU145 and PC-3 prostate cancer cell lines. In addition, CUR MT NPs were characterized in terms of particle size, zeta potential, drug entrapment efficiency (EE), polydispersity index, drug loading (DL), stability studies, and release study.

A. Materials and Methods

Materials and Cell Culture.

Curcumin was purchased from Adipogen Corporation (San Diego, Calif.). PBS was purchased from ATCC (Manassas, Va.). D-alpha-tocopheryl polyethylene glycol 1000 succinate, kollisolv MCT70, poloxamer 188, tween 20 and tween 80 were purchased from BASF (Ludwigshafen, Germany). Migloyl 812, migloyl 829, imwitor 491, imwitor 900K and imwitor 960 were obtained from Cremer (Eatontown, N.J.). Microcon Y-100 with MWCO 100 kDa was purchased from Millipore (Bedford, Mass.). Ethanol USP grade was purchased from Pharmco-AAPER (Brookfield, Conn.). Labrafac, Compriltol 888, labrasol, Gelucire 44/14 were obtained from Gattefossé (Saint Priest Cedex, France). Phosphoric acid 85 wt % was purchased from Sigma-Aldrich (St. Louis, Mo.). Acetonitrile, HPLC grade and methanol were purchased from Fisher Scientific (Pittsburgh, Pa.). The sensitive prostate cancer cell lines, PC3 and DU145, were obtained from ATCC. The resistant prostate cancer cell lines, PC and DU145, the University of Michigan. Cells were maintained in RMPI1640 supplemented with 10% fetal bovine serum (FBS) and 1% penicillin-streptomycin. Cells were cultured at 37° C. in humidified incubator with 5% CO2 and maintained in exponential growth phase by periodic subcultivation. Resistant cell lines were treated with 5 nM docetaxel in DMSO.

Screening of Oils and Surfactants.

These studies were performed to test the solubility in oils and surfactants. The solubility of CUR was determined in oils (Migloyl 812 N, Migloyl 829, Imwitor 491, Kollisolv MCT70, labrafac, Imwitor 900K, Imwitor 960 and Campriltol 888) and surfactants (Tween 20, Tween 80, Labrasol, Poloxamer 188, TPGS and Gelucire 44/14). Oils and surfactants were accurately weighed into glass vials. Excess amounts of CUR were added into glass vials containing vehicles. The vials were stirred on the hot plate maintained at 65° C. for 3 min. The drug solubility (w/w) was calculated.

Preparation of CUR Nanoparticles.

Nanoparticles were prepared by an emulsion method as described in Example 1. Defined amount of Migloyl 812 and TPGS with a 1:1 (w/w) ratio and heated to 35° C. Two (2) ml of filtered and deionized (D.I.) water pre-heated at 65° C. was added into the mixture of melted oils and surfactants. The mixture were stirred for 20 min then cool down to room temperature. To prepare CUR MT NPs, 100 μL of 4 mg/ml of curcumin dissolved in ethanol was directly added to the melted oil and surfactant and ethanol was removed by N₂ stream prior to initiating the process described above. Particle size and size distribution of NPs were measured using N5 Submicron Particle Size Analyzer (Beckman). Ten microliters of curcumin nanoparticles were diluted with 1 ml of D.I. water to reach within density range required by the instrument, and particle size analysis was performed at 90° light scattering at 25° C.

Physiochemical Characterization of CUR Nanoparticles. Particle Size and Zeta Potential Measurement.

Dynamic light scattering was used to determine the particle size of blank NPs and CUR MT NPs by using Beckman Coulter (Delsa Nano HC Particle Analyzer, California, USA). Particle size of nanoparticles were analyzed as described above. Ten microliter of blank NPs and CUR MT NPs were diluted with 1 mL of D.I. water and added 10 μL of PBS buffer (pH 7.4) for measurement of zeta potentials.

Determination of Drug Loading (DL) and Entrapment Efficiency (EE).

The concentrations of curcumin were quantified by HPLC using an Inertsil ODS-3 column (4.6 mm×150 mm, 5 μm particle size, GL Sciences Inc, CA, USA). The chromatography conditions were as follows: the mobile phase consisted of 0.1% ortho phosphoric acid and acetonitrile (40:60, v/v) at a flow rate of 1.0 mL/min, and the effluent was detected at 420 nm. The mobile phases were degas sed before use. The injection volume was 30 μl, and the analysis time was 6.5 min per sample. The HPLC method was validated for the linear ranger, limitation of detection, accuracy and precision. The retention time of CUR was about 6.5 min. For curcumin standard curve, curcumin was dissolved in methanol. The curve was found to be linear in the concentration range 0.1-50 μg/mL and r=0.998. To quantify curcumin in NPs, 1 part of curcumin NPs in water were dissolved in 8 parts of methanol. Drug loading and entrapment efficiencies were determined by separating free CUR and curcumin-loaded NPs using Microcon Y-100, and then measuring curcumin NP-containing supernatants. To ensure mass balance, the filtrates were also assayed for curcumin. The drug-loading content and drug entrapment efficiency were calculated based on the following formula:

% drug entrapment efficiency (EE)=[drug entrapped in NPs/total drug added into NP preparation]×100% (w/w)

% drug loading (DL)=[drug entrapped in NPs/weight of oil]×100% (w/w)

Particle Size Stability of NPs in 4° C. and 37° C.

The physical stability of curcumin lipid based nanoparticle suspension was evaluated over storage at 4° C. for five months. The stability of all NP suspensions was also analyzed at 37° C. in 10 mM PBS, pH 7.4 by adding 100 μL NP suspension to 13 mL PBS buffer with water-bath shaker mixing at 150 rpm. At each time interval, 1 mL aliquots were removed and allowed to equilibrate to room temperature before measuring particle sizes.

In Vitro Release Studies.

Curcumin release studies (n=3) were completed at 37° C. using PBS buffer as release medium. 200 μl of CUR MT NPs were added into 20 mL 1×PBS and shaken at speed of 135 rpm at 37° C. 500 μL from previous sample were added into centrifugal filter and centrifuged at 14,000 rpm. 200 μL of MeOH were added into centrifugal filter and reverse spinning to collect CUR MT NPs. The solution was then vortex and transferred into insert of HPLC vial. The filtrated solution of CUR MT NPs were transferred into insert of HPLC vial and particle size of CUR MT NPs were measured. The concentration of CUR was determined by using HPLC assay.

In Vitro Cytotoxicity Studies.

The cytotoxicity of CUR MT NPs was tested in DU145 and PC-3 human prostate cancer cell lines using MTT [3-(4,5-dimethylthiazol-2-yl)-2,5-diphenyltetrazolium bromide] assay. Cells were seeded into 96-well plates at 7,000 cells/well and incubated 37° C. for 24 hr under 5% CO₂. After overnight incubation, cells were treated with drug concentration ranging from 0-27 μM for CUR, CUR MT NPs, and blank NPs. Due to low water solubility, CUR was dissolved with DMSO. After 72 hr of incubation, the medium was removed from each well and replaced with 90 μL of DMEM medium without phenol read and MTT (10 μL of 5 mg/mL in PBS). After incubating for 4 hr at 37° C. with 5% CO₂, 0.04 N HCl/isopropanol was added to each well and incubated for 5 min to dissolve formazan crystals formed by viable cells. The absorbance was measured at a test wavelength of 570 nm and a reference wavelength of 650 nm in a microplate reader (Beckman Coulter, Inc.).

Statistical Analysis.

Statistical comparisons were made with ANOVA followed by pairwise comparisons using Student's t test using GraphPad Prism version 5.0 software. Results were considered significant at 95% confidence interval (p<0.05)

B. Results

Selection of Oils and Surfactants.

As shown in Table 14, CUR was found to have the highest solubility in imwitor 960 for lipid and Tween 20 for drug surfactant. Migloyl 812 was used to prepare lipid-based NPs. Tweens and TPGS are nonionic surfactant which can enhance solubility of poorly water soluble drug. However, due to toxicity associated with the uses of Tweens 20, TPGS was selected for this study. In addition, TPGS was selected due to the advantages in its ability to suppress multi-drug resistance (MDR) by inhibiting P-glycoprotein activity (Lawrence and Rees, 2000; Araya et al., 2005 and Cho, et al., 2014). Imwitor were eliminated due to the formation of highly crystalline structure with a perfect lattice that lead to drug expulsion (Hou et al., 2003).

Preparation and Characterization of CUR LNPs.

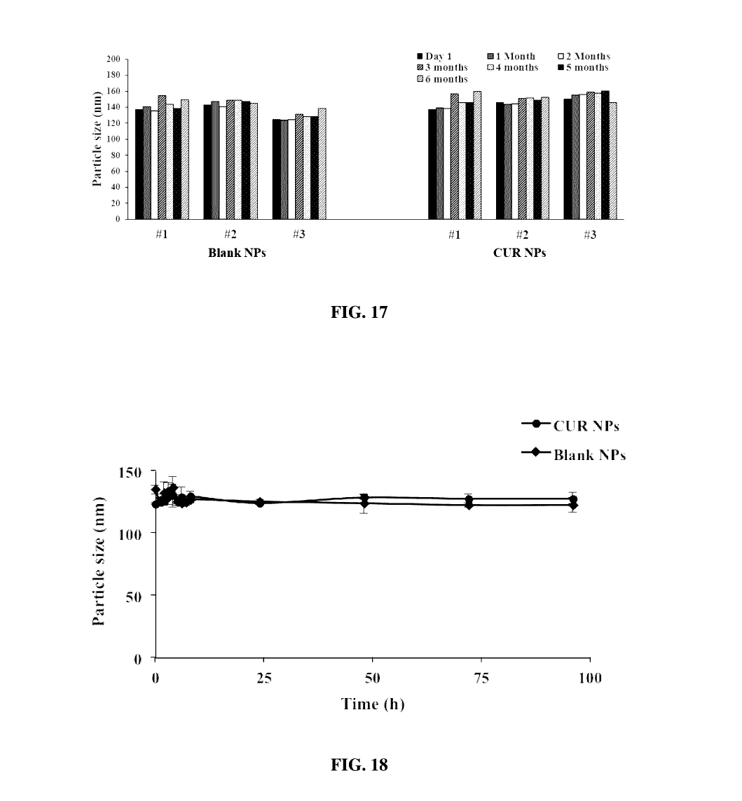

Curcumin-loaded nanoparticles were engineered by an O/W emulsion method. The CUR MT NPs were formed after cooling down to room temperature. The mean particle size of CUR MT NPs showed size ranging from 110 to 140 nm with an average particle size of 125 nm. The nanoparticle sizes were similar to blank NPs, which was around 140 nm (Table 15). The particle sizes less than 200 nm showed better distribution in tumors than larger nanoparticles (He et al., 2010). Polydispersity index (P.I.) demonstrated the narrow size distribution of CUR MT NPs. The zeta potentials of blank NPs and CUR MT NPs were −36.28 mV and -39.96 mV, respectively. The entrapment efficiency and drug loading were 83.9% and 3.15%, respectively. The physical stability of blank NPs and CUR MT NPs were monitored in both long-term and short-term storages. Blank NPs and CUR MT NPs stored as liquid suspension at 4° C. over 6 months in the long-term study and at 37° C. in PBS buffer under physiological condition in the short-term study. Blank NPs and CUR MT NPs stored as liquid suspension at 4° C. remained stable for several months and showed no significant change in particle sizes (FIG. 17). The stability of blank NPs and CUR MT NPs at PBS buffer was evaluated up to 96 hr to stimulate maximum persistence in body temperature (FIG. 18). No significant changes in particle sizes were observed for both blank NPs and CUR MT NPs preparation incubated at 37° C. in physiological pH 7.4.

In Vitro Release of Curcumin from Nanoparticles.

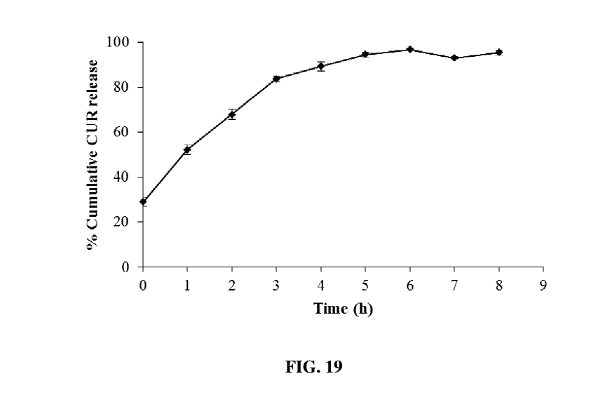

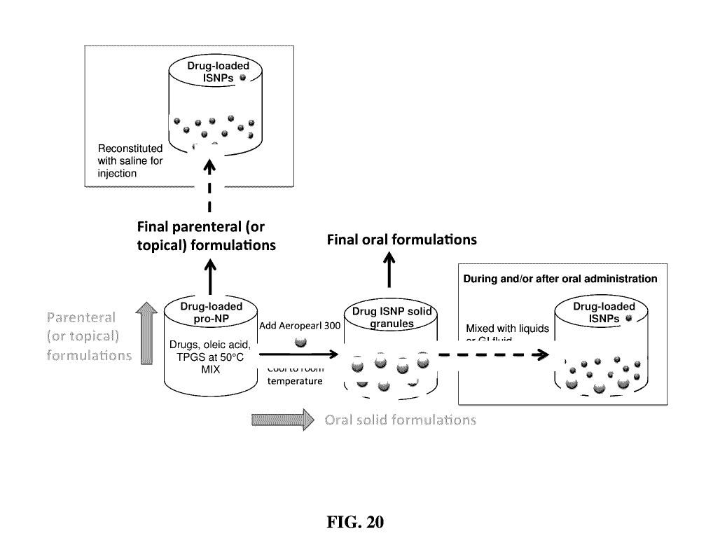

The in vitro release profile of CUR MT NPs were investigated in PBS buffer at 37° C. The cumulative curcumin release from CUR MT NPs is shown in FIG. 19. Curcumin exhibited the initial burst release at 29%. After 5 hours, the cumulative drug release was nearly complete.

In Vitro Cytotoxicity Studies.

The inventor studied cytotoxicity of curcumin in sensitive and resistant of DU145 and PC-3 cell lines by using standard MTT assay. As shown in Table 16, Free CUR showed same cytotoxicity in sensitive and resistant of PC-3 cell lines (p>0.05). In contrast, the IC₅₀ of free CUR was 1.4-fold higher in resistant DU145 than that in sensitive DU145. In a comparison, CUR MT NPs were 2-fold more potent than free CUR in all tested cell lines. Importantly, CUR MT NPs showed no significantly different IC₅₀s in sensitive and resistant PC-3 and DU145 cell lines. Blank MT NPs were safe and did not show cytotoxicity until 2500 nM.

C. Discussion

Curcumin (CUR) has been shown strong cancer preventive activity in animals (Ratz-Lyko et al., 2014; Aggarwal and Shishodia, 2006 and Aggarwal and Sung, 2009). CUR has an extremely safe profile in both animals and humans (Hatcher et al., 2008 and Parveen and Sahoo, 2008), and is “Generally Recognized as Safe” by the Food and Drug Administration. It has pleiotropic properties that modulate numerous targets including proteins (i.e., tubulin), transcription factors, growth factors and their receptors, cytokines, enzymes, and genes regulating cell proliferation and apoptosis (Shishodia et al., 2007; Wilken et al., 2011 and Anand et al., 2007). Because of this multi-targeted behavior, CUR performing a wide spectrum of actions will be a potential therapeutic agent to overcome resistant PCa caused by multiple mechanisms. CUR has been studied as a therapeutic or prevention agent in many clinical trials for various cancers with little success. For example, a clinical study comprised of 15 patients with colorectal cancer showed the cancer was nonresponsive to CUR at a daily dose of 3.6 g (4 months). So far all clinical results from oral administration of CUR have revealed very poor bioavailability. The studies concluded that while CUR exhibits anticancer effects at a concentration of 5-30 μM in cancer cells, achieving these concentrations at the tumor site in humans has not been accomplished due to its low bioavailability and higher metabolic activity. Therefore, CUR must be formulated in such a way that it can overcome these critical issues. In the inventor's view, encapsulation of CUR in nanoparticles (NPs) with possible targeting moieties is an essential approach to increase CUR concentration in the tumor site, consequently providing superior anticancer activity for cancer therapy. The inventor prepared nanoformulation of CUR based on an oil-in-water method. TPGS and curcumin can together inhibit P-gp ATPase, or reducing P-gp expression in the intestinal epithelium (Xie et al., 201). Thus, TPGS was used in this study as a P-gp inhibitot to assist CUR to overcome drug resistance.

Our LNPs with and without CUR did not significantly change in mean diameters for up to 6 months. It had been reported Neves et al that liquid lipid Migloyl 812 reduces particle crystallinity, conferring better stability and suitable for controlled release (Neves et al., 2013). Thus, the optimal of long-term stability of CUR MT NPs could result from the use of liquid Migloyl 812 in the NPs. All of NPs were smaller than 160 nm. Studies have been suggested that particles between 100 to 200 nm are desired for adhesion to and interaction with cells. It can also enhance intracellular uptake and retention effect of NPs (Kulkami and Feng, 2013 and Cho et al., 2008). P.I. were <0.25 for both blank MT NPs and CUR MT NPs, demonstrating the narrow size distribution of the NPs. Particles with zeta potential value larger than +30 mV or smaller than −30 mV are normally considered stable for colloidal dispersions (Mitri et al., 2011). Moreover, studies showed that the particles sizes with positive charge facilitate plasma protein adsorption due to opsonisation and they are susceptible to RES clearance. Negative charge can eliminate protein adsorption and RES clearance (Gessner et al., 2000). The zeta potential of CUR MT NPs were −36.28 mV, which is favor for NP stability their bioactivities. CUR MT NPs showed a relatively rapid release. The rapid release of CUR from the NPs could result from the low solubility of CUR in Miglyol 812, leading to weak interaction between Miglyol 812 and CUR. In future studies, the inventor will further optimize the NP composition to use another liquid oils to replace Miglycol 812 to modify the release profile. Overall, the inventor developed CUR MT NPs that showed appropriate properties to potentially deliver CUR to prostate tumors.

To test potential of CUR MT NPs to overcome drug resistance in CRPC, the inventor selected resistant and sensitive DU145 and PC-3 cell lines for cytotoxicity studies. PC3 and DU145 are CRPC. Deeb et al reported the usage of free CUR to sensitize hormone-resistant DU145 and PC-3 cells (Deeb et al., 2006). In these studies, free CUR showed the same IC50s in both sensitive and resistant PC3 cells, indicating that free CUR overcame hormone resistance as well as drug resistance in PC3. The microtubule dynamics is important for paclitaxel resistance because the target of paclitaxel is the microtubule. Alterations of microtubule formation, down-regulation of microtubule-related genes, alterations in tubulin composition expression, and up-regulation apoptosis genes were identified in the resistant PC-3 cells. However, P-gp overexpression is an important factor in resistant PC-3 cells. Thus, free CUR has potential to overcome the drug resistant mechanisms present in PC3 cells. Differently, free CUR showed higher IC50 in resistant DU145 cells compared to sensitive DU145 (Table 14). Indeed, P-gp overexpression is the only main cause of resistance DU145 cells. Thus, similar to previous BTM NPs, the MT NPs also overcame P-gp-mediated resistance in DU145. There are controversial reports about if CUR is a P-gp substrate. According to the results, CUR could be a P-gp substrate and the MT NPs helped CUR to overcome P-gp in resistant DU145. Moreover, CUR MT NPs were 2-fold more potent than free CUR in all tested cell lines, demonstrating that the NPs enhanced CUR cytotoxicity. Importantly, CUR MT NPs showed no significantly different IC₅₀s in sensitive and resistant PC-3 and DU145 cell lines, demonstrating that CUR MT NPs completely overcame the mechanisms not only in PC-3 but also in DU145 and the NP formulation enhanced CUR to overcome resistance in DU145. Taken together, this study demonstrates that the potential to use CUR MT NPs as a novel therapeutic agent to treat drug resistance in CRPC. To the best of the inventor's knowledge, there are no reports in literatures to use CUR MT NPs to overcome drug resistance in prostate cancer.

CUR MT NPs composed of Miglyol 812 and TPGS were successfully prepared by an emulsion method. CUR MT NPs had particle size of 124 nm with the EE % of 91.6%. The particle size of CUR MT NPs was stabled at 4° C. for 6 months and showed no significant change in particle size in a physiological condition for 96 hours. Cytotoxicity studies showed that CUR MT NPs overcame drug resistant in both PC3 and DU145 cell lines. CUR has been used for cancer therapy and prevention, and also used as a chemosensitizer to combine with doxorubicin and paclitaxel to overcome drug resistance in various cancers. However, to the best of the inventor's knowledge, this is the first report to use CUR NP itself as a novel therapeutic agent to overcome resistant CRPC.

TABLE 14
Soluble of Curcumin in oils and surfactants

| Oil | Solubility (w/w) | Surfactant | Solubility (w/w) |
|---|---|---|---|
| Migloyl 812 N | 0.0153 | Tween 20 | 0.166 |
| Migloyl 829 | 0.0210 | Tween 80 | 0.141 |
| Imwitor 491 | 0.0261 | Labrasol | 0.173 |
| Kollisolv MCT | 0.0179 | Poloxamer 188 | 0.153 |
| 70 | | | |
| Labrafac | 0.0120 | TPGS | 0.164 |
| Imwitor 900K | 0.0512 | Gelucire 44/14 | 0.153 |
| Imwitor 960 | 0.0488 | | |
| Campriltol 888 | 0.0160 | | |

TABLE 15
Physiochemical properties of Curcumin and Blank lipid based nanoparticles
(n = 3)

| | | | | | | % Drug |
|---|---|---|---|---|---|---|
| | Theoretical | Mean^{a} | | Zeta | % Drug | loading |
| | Loading | diameter | | Potential | Entrapment | (w/w, |
| Formulations | (μg/ml) | (nm) | P.I. | (mV) | Efficiency | drug/oil) |
| CUR MT NPs | 150 | 124.8 ± 7.6 | 0.186 ± 0.016 | −36.3 ± 2.4 | 83.9 ± 5.3 | 3.15 |
| Blank NPs | n/a | 139.0 ± 2.4 | 0.220 ± 0.022 | −39.1 ± 0.7 | n/a | n/a |

^{a}The data are presented as the mean of the mean particle size of nanoparticles in different batches ± SD (n = 3).

TABLE 16
IC₅₀ values (μM) of CUR, CUR MT NPs, and blank NPs incubated in PC-3 and
DU145 cell lines for 72 hr using MTT assay (n = 3)

| Cancer cells | DTX (nM) | CUR (uM) | CUR NPs (uM) | Blank NPs (uM) |
|---|---|---|---|---|
| PC-3 sensitive | 7.0 ± 0.4^{#} | 7.9 ± 0.3* | 3.9 ± 0.4** | 2563.0 ± 149.1 |
| DU-145 sensitive | 6.8 ± 0.5^{##} | 7.2 ± 0.4^{###} | 5.1 ± 0.3*** | 2767.0 ± 180.0 |
| PC-3 resistance | 75.3 ± 2.7^{#} | 7.7 ± 0.1* | 4.3 ± 0.3** | 2837.0 ± 619.9 |
| DU-145 resistance | 870.3 ± 85.4^{##} | 10.0 ± 0.5^{###} | 4.9 ± 0.4*** | 5559 ± 681.3 |

Free CUR was used as a standard for comparing with CUR MT NPs.
^{#}, ^{##}, ^{###}p < 0.05;
*, **, ***p > 0.05.

### Example 8

Clindamycin is an antibiotics used to treat moderate and severe acne by topical and oral administration. Clindamycin free base is poorly water-soluble. The commercially available clindamycin topical formulations, such as gels and lotions, are made of clindamycin salts, such as its phosphate and hydrochloride salt. The permeability of these commercial clindamycin products is limited because of high water solubility of these salts, leading to low lipid permeability in skin. Thus, the lower bioavailability of these conventional topical formulations is considered an issue for effective treatment. Moreover, another major problem associated with conventional clindamycin salt topical formulation is the drug's side effects such as cutaneous irritation, dryness, peeling and scaling. Retinoic acid is a drug used to treat acne. The combination of retinoic acid and antibiotics is an innovative approach for acne treatment. However, retinoic acid is followed by side effects, such as erythema and irritation. Moreover, retinoic acid is a poorly water-soluble drug. Oleic acid has been demonstrated to modulate the closure of surgically induce skin wounds as well anti-inflammatory characteristics. TPGS is a non-ionic surfactant which possesses a better capacity to dissolve water-insoluble drugs and is less toxic. TPGS has been used as a penetration enhancer for transdermal delivery. The inventor's OT NPs combining the benefits of oleic acid and TPGS will promote drug delivery to skin. Therefore, the inventor has developed clindamycin/retinoic acid OT gels for topical acne therapy. The OT NPs solved the solubility issues for retinoic acid and clindamycin free base. By using clindamycin free base, the inventor will improve the permeability of clindamycin and consequently improve the drug bioavailability. By encapsulating both drugs into the OT NPs, the NPs will prevent drugs from the contact with skin as well as facilitating the skin penetration. Therefore, the side effects of the drugs will be reduces and the bioavailability will be improved. With the help of oleic acid, the clindamycin/retinoic acid OT gels will provide superior antibacterial effect.

Methods.

The inventor prepared clindamycin/retinoic acid OT ISNPs as described in Example 2. Briefly, 20 mg clindamycin free base, 1 mg retinoic acid, 30 mg oleic acid and 60 mg TPGS were mixed in a glass vial at 60° C. for 10 min. Then, the mixture was moved to a cool plate and continually stirred to room temperature. After preparation, the inventor obtained the clindamycin/retinoic acid OT pro-ISNP that is semi-solid (soft wax). Then, a gel agent (1050 mg), such as Carbopol 941 and Carbopol 1342, was prepared and mixed with clindamycin/retinoic acid OT pro-ISNP. The drug-loaded gel was reconstituted with water to measure particles size, polydispersity index and size distribution. The gel could be directly applied to acne for the treatment.

Results.

After reconstituted with water, the OT pro-ISNP formed the ISNPs. The particle size of clindamycin/retinoic acid ISNPs was 310 nm with a narrow size distribution (polydispersity index=0.26). According to the observation, clindamycin free base and retinoic acid quickly dissolved in melted TPGS and oleic acid, indicating great miscibility of the components that could result in high entrapment efficiency of drugs. The inventor is currently measure the entrapment efficiency, long-term stability and skin permeability using an in vitro human skin permeation experiment based on glass Franz diffusion cells and an ex vivo drug permeation model based on rat skin.

All of the compositions and methods disclosed and claimed herein can be made and executed without undue experimentation in light of the present disclosure. While the compositions and methods of this disclosure have been described in terms of certain embodiments, it will be apparent to those of skill in the art that variations may be applied to the compositions and methods and in the steps or in the sequence of steps of the methods described herein without departing from the concept, spirit and scope of the disclosure. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the disclosure as defined by the appended claims.

### V. REFERENCES

The following references, to the extent that they provide exemplary procedural or other details supplementary to those set forth herein, are specifically incorporated herein by reference.
Abdelwahed et al., Adv. Drug Deliv. Rev., 58:1688-1713, 2006
Abidi and Cabazitaxel, J Pharmacol Pharmacothe., 4:230-237, 2013.
Adefolalu and Nkosi, Diseases 1:18-35, 2013.
Aggarwal and Shishodia, Biochem Pharmacol., 71:1397-1421, 2006.
Aggarwal and Sung, Trends Pharmacol Sci., 30:85-94, 2009.
Anand et al., Cancer Lett., 267:133-164, 2008.
Anand et al., Mol Pharm., 4:807-818, 2007.
Antonaraski and Armstrong, Prostate Cancer Prostatic Dis., 14:192-205, 2011.
Antonelli and Turriziani, International journal of antimicrobial agents, 40: 95-102, 2012.
Araya et al., Int J Pharm., 305:61-74, 2005.
Balakrishnan et al., Eur J Pharm Biopharm., 72:3:539-545, 2009.
Baldwin et al., Sensors (Basel), 11:5:4744-4766, 2011.
Becker, Expert Rev Anti Infect Ther., 1:3:403-413, 2003.
Berthold et al., J Clin Oncol., 26:242-245, 2008.
Bissery et al., Cancer Res., 51:4845-4852, 1991.
Boffito et al., Antivir Ther., 13:7:901-907, 2008.
Chearwae et al., Pharmacol, 57:376-388, 2006.
Chesney, M., Clin Infect Dis 30:S171-S176, 2000.
Cho et al., Clin Cancer Res., 14:1310-1316, 2008.
Cho et al., Mol Pharm, 10(6):2093-2110, 2013.
Cho, et al., Int J Nanomedicine, 9:495-504, 2014.
Chun and Lee, Biochem Biophys Res Commun., 315:771-779, 2004.
Clayden, HIV HCV TB Pipeline Report, November, 2013.
Collnot et al., Mol Pharmaceut., 7:642-651, 2010.
Das and Chaudhury, AAPS Pharm Sci Tech., 12:62-76, 2011.
De Jaeghere et al., J Control Release., 68:2:291-298, 2000.
Deeb et al., J Exp Ther Oncol., 5:81-89, 2006.
Deeb et al., Mol Cancer Ther., 3:803-812, 2004.
Desai, The AAPS Journal, 14(2):282-295, 2012.
Destache et al., BMC Infect. Dis. 9:198-206, 2009.
Dong and Mumper, Nanomedicine, 5:597-615, 2010.
Dong et al., Eur J Pharm Biopharm., 72:9-17, 2009.
Dong et al., Oncogene, 21:6510-6519, 2002.
Duhem et al., J Controlled Release, 182:33-34, 2014.
Durand and Flexner, Clin Pharmacol Ther., 93:5:382-384, 2013.
Fasinu et al., Biopharmaceutics & drug disposition, 32:185-209, 2011.
Feng et al. Int J Nanomedicine., 6:2545-2556, 2011.
Gessner et al., Int J Pharm., 196:245-249, 2000.
Grama et al., Curr Opin Colloid In., 16:238-245, 2011.
Guo et al., Eur J Pharm Sci., 49(2):175-186, 2013.
Gupta et al., Recent Pat Drug Deliv Formul., 4:1:82-89, 2010.
Hatcher et al., Cell Mol. Life Sci., 65:1631-1652, 2008.
He et al., Biomaterials, 31:3657-3666, 2010.
Hennenfent and Govindan, Ann Oncol., 17:5:735-739, 2006.
Hippalgaonkar et al., AAPS Pharm Sci Tech, 11(4):1526-1540, 2010.
Ho et al., Drug Delivery and Translation Research 5:531-39, 2015.
Hou et al., Biomaterials, 24:1781-1785, 2003.
Jin et al., PLoS ONE, 9(11):1-12, 2014.
Johnson and Neilands, AIDs and Behavior 11:575-585, 2007.
Jong et al., Int J Nanomedicine, 3:133-149, 2008.
Kaletra, North Chicago (Ill.): Package insert. Distributed by AbbVie, Inc., November 2015.
Kawai et al., Cancer Lett, 150:147-153, 2000.
Khan et al., Expert opinion on drug delivery, 9:1305-1317, 2012.
Koudelka et al., J Control Release, 207:59-69, 2015.
Kroon et al., Cancer Treat Rev., 40:578-584, 2014.
Kulkami and Feng, Pharm Res., 30:2512-2522, 2013.
Lawrence and Rees, Adv Drug Deliver Rev., 45:89-121, 2000.
Li et al., Int J Nanomedicine, 6:897-904, 2011.
Limtrakul et al., Mol Cell. Biochem., 296:85-95, 2007.
Luo et al., J Control Release, 147(2):278-288, 2010.
Ma et al., Int J Nanomedicine, 6:2679-2688, 2011.
Madgulkar et al., AAPS Pharm Sci Tech, 10:574-581, 2009.
Mahmoud et al., AAPS Pharm Sci Tech., 10:1:183-192, 2009.
Margolis and Marta, Current Opinion in HIV & AIDS, 10:246-252, 2015.
Markman et al., Adv Drug Deliv Rev., 65:1866-1879, 2013.
Matés et al., Curr Med Chem., 18:2315-2338, 2011.
Mazzarino, J Colloid Interface Sci., 370: 58-66, 2012.
Médecins Sans Frontières, ed., Antiretroviral Sequencing Meeting Report, 2011.
Mishra and Palanivelu, Ann Indian Acad Neurol., 11:13-19, 2008.
Mitri et al., Int J Pharm., 414:267-275, 2011.
Mizokami et al., Prostate, 67:955-967, 2007.
Mosallaei et al., J Pharm Sci., 102(6):1994-2004, 2013.
Nassar et al., Cancer Res., 71(8):3018-3028, 2011.
National Institutions of Health, ed., Guidelines for the use of antiretroviral agents in pediatric HIV infection, 2013.
Nelson et al., 7th IAS Conference on HIV Pathogeness, Treatment and Prevention, 2013.
Neves et al., Int J Nanomedicine, 8:177-187, 2013.
Nielsen, et al., Eur J Pharm Biopharm., 69:2:553-562, 2008.
Norris et al., Commun Oncol., 7:425-428, 2010.
Ohsima et al., Int J Pharm 377:180-84, 2009.
O'Neill et al., Mol Cancer, 10 doi:10.1186/1476-4598-10-126, 2011.
Ostacolo et al., J Control Release, 148(2):255-263, 2014.
Parveen and Sahoo, J Drug Target, 16:108-23, 2008.
Petrylak, Rev Urol., 8 Suppl 2:S48-S55, 2006.
Pham et al., J. Control. Release 226:88-97, 2016.
Popat et al., Colloids and Surfaces B: Biointerfaces, 117:520-527, 2014.
Pouton, Eur J Pharm Sci., 29:3-4:278-287, 2006.
Ratz-Lyko et al., Indian J Pharm Sci., 76:374-378, 2014.
Ringel and Horwitz, J Natl Cancer Inst., 83(4):288-291, 1991.
Rodriguez-Spong et al., Mol Pharm., 5:6:956-967, 2008.
Sanna and Sechi, Maturitas, 73:27-32, 2012.
Schneider et al., Dermatoendocrinol 1:197-206, 2009.
Shishodia et al., Curr Prob Cancer, 31:243-305, 2007.
Shoba et al., Planta Med., 64:353-356, 1998.
Shuter, J., J Antimicrob Chemother 61:769-773, 2008.
Siegel et al., CA Cancer J Clin., 64:9-29, 2014.
Singer and Srinivasan, Urol Oncol-Semin Ori., 30:S15-S19, 2012.
Spreen et al., Curr Opin HIV AIDS 8:565-571, 2013.
Sun et al., Colloids Surf B Biointerfaces, 111C:367-375, 2013.
Suzuki et al., Prostate Cancer Prostatic Dis, 10:301-306, 2007.
Taha et al., J Drug Target, 17:6:468-473, 2009.
Tannock et al., N Engl J Med, 351:1502-1512, 2004.
Tonnesen et al., Int J Pharm., 2449:127-135, 2002.
Tuleu and Breitkreutz, Eur J Pediatr, 172:6:717-720, 2013.
upta et al., ISBN Pharm., 2013:848043, 2013.
Van Brussel and Mickisch, Onkologic 26, 26:175-181, 2003.
Verderio et al., Biomacromolecules, 14:672-682, 2013.
Vrignaud et al., International journal of pharmaceutics, 487:270-273, 2015.
Walsh and Mills, Ther Deliv. 4(1):21-25, 2013.
Walsh, Eur Industrial Pharmacy, 13:14-16, 2012.
Wang et al., J Pharmaceut Biomed., 15:1867-1876, 1997.
Weiszhar et al., Eur J Pharm Sci., 45:492-498, 2012.
Wilken et al., Mol Cancer, 10:12, 2011.
World Health Organization, Consolidated guidelines on the use of antiretroviral drugs for treating and preventing HIV infection, 2013.
World Health Organization, Updated list of missing drug formulations for HIV treatment. 2011.
World Health Organization, 26:1:15-23, 2012.
World Health Organization, HIV/AIDS (Global Health Observatory Data), 2016.
Xie et al., J Agri Food Chem., 59:9280-9289, 2011.
Zhang et al., J Pharm Sci., 103:2177-2183, 2014.
Zhu et al., Biomaterials, 35:2391-2400, 2014.
Zidan, Pharmaceutical development and technology, 1:1-11, 2016.

1. A pharmaceutical composition comprising:
(a) a therapeutic agent;
(b) a pharmaceutically acceptable oil phase;
(c) a surfactant,
wherein the composition is formulated as a pro-nanoparticle composition.

2. The pharmaceutical composition of claim 1, wherein the pharmaceutically acceptable oil phase is a fatty acid or a mixture of a fatty acid, or an unsaturated fatty acid.

3. The pharmaceutical composition of claim 2, further comprising a pharmaceutically acceptable polymer.

4-6. (canceled)

7. The pharmaceutical composition of claim 1, wherein the pharmaceutically acceptable oil phase is a composition comprising one or more compounds of the formula: 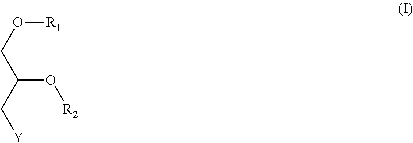
wherein:
R₁ and R₂ are each independently hydrogen or —C(O)—R₄; and
Y is hydrogen, hydroxy, or —OC(O)—R₄; wherein:
R₄ is alkyl_{(C1-25)}, alkenyl_{(C1-25)}, alkynyl_{(C1-25)}, or a substituted version of any of these groups; or a group of the formula: —C(O)—X—C(O)H, wherein X is an alkanediyl_{(C1-12)} or substituted alkanediyl_{(C1-12)};
provided that R₁, R₂, and Y are not all hydrogen.

8-10. (canceled)

11. The pharmaceutical composition of claim 1, wherein the pharmaceutically acceptable oil phase is a naturally derived liquid oil.

12. (canceled)

13. The pharmaceutical composition of claim 1, wherein the surfactant has a hydrophilic-lipophilic balance (HLB) of from about 6 to about 20.

14. (canceled)

15. The pharmaceutical composition of claim 13, wherein the surfactant is conjugated to a polyethylene glycol, a cell-targeting ligand, a small molecule, a peptide, a protein, or a carbohydrate.

16-19. (canceled)

20. The pharmaceutical composition according to claim 1, wherein the surfactant further comprises a co-surfactant.

21-24. (canceled)

25. The pharmaceutical composition according to claim 1, wherein the pharmaceutical composition comprises one therapeutic agent, a mixture of two or more therapeutic agents.

26. (canceled)

27. The pharmaceutical composition according to claim 1, wherein the therapeutic agent is a substantially water insoluble or lipophilic therapeutic agent or is a water-soluble therapeutic agent.

28-45. (canceled)

46. The pharmaceutical composition according to claim 1, wherein the pharmaceutical composition further comprises an absorption agent.

47-50. (canceled)

51. The pharmaceutical composition according to claim 1, wherein the pharmaceutical composition is essentially free of water.

52. The pharmaceutical composition according to claim 1, wherein the pharmaceutical composition is reconstituted into a liquid thereby forming a nanoparticle.

53. The pharmaceutical composition of claim 51, wherein the liquid is water, saline, an injectable solution, juice, or other water based solution.

54. The pharmaceutical composition of claim 51, wherein the liquid is salvia or another biological fluid such that the nanoparticle is formed in vivo.

55-66. (canceled)

67. The pharmaceutical composition according to claim 1, wherein the pharmaceutical composition further comprises a pharmaceutically acceptable carrier.

68-72. (canceled)

73. A method of preparing a nanoparticle pharmaceutical composition comprising:
(a) admixing a pharmaceutically acceptable oil phase, a surfactant, and a therapeutic agent to form a first mixture; and
(b) heating the first mixture to a temperature from about 30° C. to about 100° C.

74. (canceled)

75. A method of preparing an in-situ self assembly nanoparticle pharmaceutical composition comprising:
(a) admixing a therapeutic agent, a pharmaceutically acceptable oil phase, a pharmaceutically acceptable polymer and a surfactant to form a first mixture;
(b) heating the first mixture to a temperature from about 30° C. to about 100° C. for a time period from about 1 minute to about 1 hour; and
(c) cooling the first mixture to room temperature.

76-77. (canceled)

78. A method of preparing a pharmaceutical composition comprising:
(a) admixing a therapeutic agent, a pharmaceutically acceptable oil phase, a pharmaceutically acceptable polymer and a surfactant to form a first mixture;
(b) heating the first mixture to a temperature from about 30° C. to about 100° C. for a time period from about 1 minute to about 1 hour;
(c) admixing an absorption agent, a base or a gelling agent to the first mixture to obtain a second mixture;
(d) heating the second mixture to a temperature from about 30° C. to about 100° C. for a time period from about 1 minute to about 1 hour; and
(e) cooling the second mixture to room temperature.

79-118. (canceled)

119. A method of treating a disease or disorder comprising administering to a patient in need thereof a therapeutically effective amount of a pharmaceutical composition according to claim 1.

120-137. (canceled)
